(19) United States
(12) Patent Application Publication
(10) Pub. No.: US 2024/0269659 A1
(43) Pub. Date: Aug. 15, 2024

(54) FRUSTRATED LEWIS PAIR-IMPREGNATED POROUS MATERIALS AND USES THEREOF

(71) Applicant: UNIVERSITY OF SOUTH FLORIDA, TAMPA, FL (US)

(72) Inventors: Shengqian MA, Tampa, FL (US); Zheng NIU, Tampa, FL (US)

(21) Appl. No.: 18/394,919

(22) Filed: Dec. 22, 2023

Related U.S. Application Data
(63) Continuation of application No. 17/623,801, filed on Dec. 29, 2021.
(60) Provisional application No. 62/869,299, filed on Jul. 1, 2019.

(51) Int. Cl. B01J 31/16; B01J 31/02; C01B 3/00; C07C 67/303; C07C 209/52; C07C 213/02; C07F 5/02; H01M 8/04082

(52) U.S. Cl. CPC B01J 31/1691; B01J 31/0275; C01B 3/0015; C07C 67/303; C07C 209/52; C07C 213/02; C07F 5/027; H01M 8/04216; B01J 2231/643; B01J 2231/645; B01J 2531/62

(57) ABSTRACT

Described herein are compositions composed of frustrated Lewis pairs impregnated in porous materials such as, for example, metal-organic frameworks, and their uses thereof. These compositions may allow new applications of frustrated Lewis pairs in catalysis by sequestering and protecting the frustrated Lewis pair within the nanospace of the porous material. Also provided are methods of hydrogenating an organic compound having at least one unsaturated functional group comprising using the compositions described herein.

### CROSS REFERENCE TO RELATED APPLICATIONS

This application is a continuation of U.S. patent application Ser. No. 17/623,801, filed Dec. 29, 2021, which is a U.S. national stage entry of International Patent Application Ser. No. PCT/US2020/040310, filed Jun. 30, 2020, which claims priority to U.S. Provisional Patent Application Ser. No. 62/869,299 filed Jul. 1, 2019, the contents of each are hereby incorporated by reference in their entireties.

### ACKNOWLEDGEMENTS

This invention was made with government support under EE0008810 awarded by the Department of Energy. The government has certain rights in the invention.

### BACKGROUND

Small molecules such as carbon dioxide and dihydrogen are abundant chemical sources on earth. Taking their thermodynamic stability into consideration, the activation of small molecules is not only of great interest from a fundamental chemistry perspective but also long has been a challenging process, particularly when transforming natural resources into industrially applicable fine chemicals. The most prominent examples of small-molecule activation reside in some biological processes, including carbon dioxide fixation by ribulose-1,5-bisphosphate carboxylase/oxygenase during the Calvin cycle in plants and dihydrogen uptake in microbes by metalloenzymes known as hydrogenases. Combining both atom economy and wide applicability, catalytic hydrogen of organic compounds containing unsaturated bonds has drawn comprehensive attention since the early 20^{th} century. Aiming at constructing factitious hydrogenase functions, organometallic studies during the past several decades have provided some insightful solutions to finding synthetic alternatives for hydrogenation, including the discovery of homogenous ruthenium and rhodium catalysts by Wilkinson as well as organo-iridium catalysts by Crabtree. Asymmetric hydrogenations were later achieved based on chiral ligand design of ruthenium and rhodium complexes by Noyori and Knowles. Heterogeneous hydrogenation catalysis is also dictated by precious noble metals (such as Pd, Pt, Ru, Rh, or Ir). These catalysts have showed high efficiency and selectivity compared to stoichiometric reductants. However, the high cost together with potential toxicity casts a shadow on their broader applications in industry, particularly in the manufacture of pharmaceuticals.

Inspired by the development of bifunctional catalysis, frustrated Lewis pairs (FLPs) as pioneered by Douglas Stephan in 2006 (Science, 2006, 314:1124-1126) are distinct from metal-catalyzed systems in both reaction pathways and functionalities. Using only p-block elements, frustrated Lewis pairs are capable of combining Lewis acidity (mainly by group III atom containing species) and Lewis basicity (mainly by group V atom containing species) in a bi-molecular or even unimolecular fashion without inducing formation of an acid-base adduct. This molecular structure has shown unique versatility in activating small molecules such as H₂, CO₂, CO, SO₂, N₂O, and NO. Over the past decade, both stoichiometric and catalytic reactions of various types have been well developed. Among the various reactions catalyzed by frustrated Lewis pairs, hydrogenation is the most comprehensively studies in terms of mechanism, substrate scope, and selection of frustrated Lewis pairs. Sharing the same generic mechanism of heterolytic H₂ cleaved followed by proton transfer and hydride transfer, a variety of substrates can undergo hydrogenation of an unsaturated bond yield the reduced product, including such transformations as reducing an olefin to an alkane, an alkyne to a Z-olefin, an imine to an amine, or even selectively converting an enone to a ketone. The concise catalytic cycle includes only four steps virtually epitomizes frustrated Lewis pair catalyst design as a process of tuning the electronic and steric properties of the building blocks. Consequently, this has led to a multitude of distinctive frustrated Lewis pair systems, the majority of which are constructed from carbon-based organic backbones. These structures are then endowed with more accessible functionalization compared to metal catalysts considering the complexity of ligand design. However, the practical application of frustrated Lewis pair-catalyzed hydrogenation is limited by the moisture sensitivity of the typically used boron-based Lewis acids. Although mitigation of Lewis acidity or enhancement of steric hindrance close to the reactive center has been used as a strategy to minimize moisture sensitivity, this inevitably leads to lower reactivity and thus requires further modifications. Other issues, such as poor reusability and lack of selectivity, have also been recognized as barrier limiting the broader use of frustrated Lewis pairs in industrial applications.

### SUMMARY

Described herein are compositions composed of frustrated Lewis pairs impregnated in porous materials such as, for example, metal-organic frameworks, and their uses thereof. These compositions may allow new applications of frustrated Lewis pairs in catalysis by sequestering and protecting the frustrated Lewis pair within the nanospace of the porous material. Also provided are methods of hydrogenating an organic compound having at least one unsaturated functional group comprising using the compositions described herein.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not descriptive.

### BRIEF DESCRIPTION OF FIGURES

FIG. 1 is scheme demonstrating a step-wise anchoring strategy for grafting a frustrated Lewis pair on open metal sites of a metal-organic framework.

FIG. 2 shows the cage structure of the representative metal-organic framework MIL-101(Cr) as can used in the present disclosure.

FIG. 3 shows the cage structure of the representative metal-organic framework PCN-333(Cr) as can used in the present disclosure.

FIG. 4 shows the cage structure of the representative metal-organic framework Tb-MesoMOF as can used in the present disclosure.

FIG. 5 shows the cage structure of the representative metal-organic framework MOF-74-III as can used in the present disclosure.

FIG. 6 shows the cage structure of the representative metal-organic framework PCN-777 as can used in the present disclosure.

FIG. 7 shows the cage structure of the representative metal-organic framework PCN-69 as can used in the present disclosure.

FIG. 8 shows the cage structure of the representative metal-organic framework PCN-600 as can used in the present disclosure.

FIG. 9 shows an additional cage structure of the representative metal-organic framework PCN-333(Cr) as can used in the present disclosure.

FIG. 10 shows the cage structure of the representative metal-organic framework CYCU-3 as can used in the present disclosure.

FIG. 11 shows the cage structure of the representative metal-organic framework Bio-MOF-100 as can used in the present disclosure.

FIG. 12 shows the cage structure of the representative metal-organic framework MOF-210 as can used in the present disclosure.

FIG. 13 shows the cage structure of the representative metal-organic framework UiO-68 as can be used in the present disclosure.

FIG. 14 is a scheme showing the de novo assembly of a covalently-linked frustrated Lewis pair within a metal-organic framework by step-wise introduction of a Lewis-base precursor and a Lewis acid precursor.

FIG. 15 is a scheme showing the construction of a metal-organic framework containing a Lewis base functionality on the organic linker ligand that is used to form a frustrated Lewis pair-impregnated metal-organic framework.

FIG. 16 is a scheme demonstrating surface modification of a metal-organic framework to form a superhydrophobic metal-organic framework as can be used in the present disclosure.

FIG. 17 is a scheme demonstrating how a superhydrophobic metal-organic framework can help facilitate water expulsion during reactions using a frustrated Lewis pair-impregnated metal-organic framework that product water as a byproduct.

FIG. 18 is a scheme that illustrates how open metal sites in frustrated Lewis pair-impregnated metal-organic frameworks can increase selectivity for 1,2-reduction in α,β-unsaturated carbonyls by binding to the carbonyl oxygen.

FIG. 19 is a scheme that illustrates how a frustrated Lewis pair-impregnated metal-organic framework composed of chiral organic linker ligands or chiral frustrated Lewis pair components can be used to affect asymmetric hydrogenations of prochiral substrates.

FIG. 20 is a scheme that illustrates hydrogen adsorption using a frustrated Lewis pair-impregnated metal-organic framework.

FIG. 21A is a scheme showing the formation of MIL-101-FP-H₂ by high-pressure introduction of hydrogen and the subsequent decompression. The frustrated Lewis pair used is a combination of B(C₆F₅)₂(Mes) and DABCO.

FIG. 21B is graph comparing the uptake of hydrogen by MIL-101-FP and MIL-101 at 298 K. An excess hydrogen adsorption isotherm is observed for MIL-101-FLP as compared to MIL-101. The y-axis is the uptake amount of hydrogen measured in cubic centimeters per gram, and the x-axis is hydrogen pressure measured in bar.

FIG. 22 is a scheme that illustrates the chemoselective hydrogenation of a substrate due to open metal sites within a frustrated Lewis pair-impregnated metal-organic framework.

FIG. 23A is a powder X-ray diffraction graph comparing MIL-101(Cr) to MIL-101(Cr)-LP, where LP is a combination of DABCO and B(C₆F₅)₃. The y-axis is intensity in arbitrary units and the x-axis is 2-theta measured in degrees.

FIG. 23B is an N₂ sorption isotherm graph comparing N₂ uptake for MIL-101(Cr) and MIL-101(Cr)-LP, where LP is a combination of DABCO and B(C₆F₅). The y-axis is the nitrogen uptake amount in cubic centimeters per gram, and the x-axis is the relative pressure.

FIG. 24A are infrared spectra comparing the frustrated Lewis pair (LP) of DABCO and B(C₆F₅)₃, MIL-101(Cr), and MIL-101(Cr)-LP. The x-axis measures wavenumbers.

FIG. 24B is an X-ray photon spectroscopy (XPS) spectrum of MIL-101. The y-axis measure intensity in arbitrary units, and the x-axis measures bonding energy in electron volts.

FIG. 24C is an X-ray photon spectroscopy (XPS) spectrum of MIL-101(Cr)-LP, where the LP is a combination of DABCO and B(C₆F₅)₃. The y-axis is intensity in arbitrary units, and the x-axis is bonding energy in electron volts.

FIG. 24D are survey X-ray photo spectroscopy (XPS) spectra of MIL-101 and MIL-101(Cr)-LP, where the LP is a combination of DABCO and B(C₆F₅)₃. The y-axis is intensity in arbitrary units, and the x-axis is bonding energy in electron volts.

FIG. 25A is a scanning electron microscopy image of MIL-101(Cr)-LP, where LP is a combination of DABCO and B(C₆F₅)₃.

FIG. 25B is a transmission electron microscopy image of MIL-101(Cr)-LP, where LP is a combination of DABCO and B(C₆F₅)₃.

FIG. 25C are high-angle dark-field imaging scanning transmission electron microscopy images of MIL-101(Cr)-LP with elemental mapping for Cr, F, and N, where LP is a combination of DABCO and B(C₆F₅)₃.

FIG. 26 is a scheme for a proposed mechanism for the reduction of imines using pinacolborane using MIL-101(Cr)-LP as a catalyst.

FIG. 27 is graph showing the recyclability of MIL-101(Cr)-LP in the reduction of imines using pinacolborane. The y-axis is the yield of the reaction, and the x-axis is the number of runs using recycled MIL-101(Cr)-LP.

FIG. 28 is a scheme demonstrating the activation process in MIL-101(Cr)-FLP-H₂, where the FLP is a combination of B(C₆F₅)₂(Mes) and DABCO.

FIG. 29A are 11B nuclear magnetic resonance spectra of MIL-101(Cr)-FLP-H₂ and FLP, where the FLP is a combination of B(C₆F₅)₂(Mes) and DABCO. The axis is the chemical shift in parts per million.

FIG. 29B are infrared spectra of MIL-101(Cr), FLP-H₂, and MIL-101(Cr)-FLP-H₂, where the FLP is a combination of B(C₆F₅)₂(Mes) and DABCO. The axis is measured in wavenumbers.

FIG. 30A is a scanning electron microscopy image of MIL-101(Cr)-FLP-H₂, where the FLP is a combination of B(C₆F₅)₂(Mes) and DABCO.

FIG. 30B is a transmission electron microscopy image of MIL-101(Cr)-FLP-H₂, where the FLP is a combination of B(C₆F₅)₂(Mes) and DABCO.

FIG. 30C are high-angle dark-field imaging scanning transmission electron microscopy images of MIL-101(Cr)-FLP-H₂ with elemental mapping for Cr, F, and N, where the FLP is a combination of B(C₆F₅)₂(Mes) and DABCO.

FIG. 31 is a theoretical model calculated with density functional theory (DFT) for the interaction of N-(tert-butyl)-3-phenylprop-2-en-1-imine with the Cr₃O(OH)—(COO)₆H₂O trimer on a MIL-101(Cr)-FLP-H₂ catalyst, where the FLP is a combination of B(C₆F₅)₂(Mes) and DABCO.

FIGS. 32A-B show the ability of a compositions described herein to adsorb hydrogen.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### General Definitions

In this specification and in the claims that follow, reference will be made to a number of terms, which shall be defined to have the following meanings:

Throughout the specification and claims the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an inhibitor” includes mixtures of two or more such inhibitors, reference to “the kinase” includes mixtures of two or more such kinase, and the like.

“Optional” or “optionally” means that the subsequently described event or circumstance can or cannot occur, and that the description includes instances where the event or circumstance occurs and instances where it does not.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements. Furthermore, when numerical ranges of varying scope are set forth herein, it is contemplated that any combination of these values inclusive of the recited values may be used. Further, ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Unless stated otherwise, the term “about” means within 5% (e.g., within 2% or 1%) of the particular value modified by the term “about.”

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

### Chemical Definitions

As used herein, the term “composition” is intended to encompass a product comprising the specified ingredients in the specified amounts, as well as any product which results, directly or indirectly, from combination of the specified ingredients in the specified amounts.

References in the specification and concluding claims to parts by weight of a particular element or component in a composition denotes the weight relationship between the element or component and any other elements or components in the composition or article for which a part by weight is expressed. Thus, in a mixture containing 2 parts by weight of component X and 5 parts by weight component Y, X and Y are present at a weight ratio of 2:5, and are present in such ratio regardless of whether additional components are contained in the mixture.

A weight percent (wt. %) of a component, unless specifically stated to the contrary, is based on the total weight of the formulation or composition in which the component is included.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valencies of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc.

The term “aliphatic” as used herein refers to a non-aromatic hydrocarbon group and includes branched and unbranched, alkyl, alkenyl, or alkynyl groups.

The term “alkyl” as used herein is a branched or unbranched saturated hydrocarbon group of 1 to 24 carbon atoms, such as methyl, ethyl, n-propyl, isopropyl, n-butyl, isobutyl, t-butyl, pentyl, hexyl, heptyl, octyl, nonyl, decyl, dodecyl, tetradecyl, hexadecyl, eicosyl, tetracosyl, and the like. The alkyl group can also be substituted or unsubstituted. The alkyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below. As described herein, “perfluoroalkyl” is an alkyl group as described herein where each hydrogen substituent on the group has been substituted with a fluorine atom. Representative but non-limiting examples of “perfluoroalkyl” groups include trifluoromethyl, pentafluoroethyl, or heptadecafluorooctyl.

The symbols Aⁿ is used herein as merely a generic substituent in the definitions below. The term “alkoxy” as used herein is an alkyl group bound through a single, terminal ether linkage; that is, an “alkoxy” group can be defined as —OA¹ where A¹ is alkyl as defined above.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This may be presumed in structural formulae herein wherein an asymmetric alkene is present, or it may be explicitly indicated by the bond symbol C═C. The alkenyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “alkynyl” as used herein is a hydrocarbon group of 2 to 24 carbon atoms with a structural formula containing at least one carbon-carbon triple bond. The alkynyl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol, as described below.

The term “aryl” as used herein is a group that contains any carbon-based aromatic group including, but not limited to, benzene, naphthalene, phenyl, biphenyl, phenoxybenzene, and the like. The term “heteroaryl” is defined as a group that contains an aromatic group that has at least one heteroatom incorporated within the ring of the aromatic group. Examples of heteroatoms include, but are not limited to, nitrogen, oxygen, sulfur, and phosphorus. The term “non-heteroaryl,” which is included in the term “aryl,” defines a group that contains an aromatic group that does not contain a heteroatom. The aryl and heteroaryl group can be substituted or unsubstituted. The aryl and heteroaryl group can be substituted with one or more groups including, but not limited to, alkyl, halogenated alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein. The term “biaryl” is a specific type of aryl group and is included in the definition of aryl. Biaryl refers to two aryl groups that are bound together via a fused ring structure, as in naphthalene, or are attached via one or more carbon-carbon bonds, as in biphenyl.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, etc. The term “heterocycloalkyl” is a cycloalkyl group as defined above where at least one of the carbon atoms of the ring is substituted with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cycloalkenyl” as used herein is a non-aromatic carbon-based ring composed of at least three carbon atoms and containing at least one double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, and the like. The term “heterocycloalkenyl” is a type of cycloalkenyl group as defined above where at least one of the carbon atoms of the ring is substituted with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, alkoxy, alkenyl, alkynyl, aryl, heteroaryl, aldehyde, amino, carboxylic acid, ester, ether, halide, hydroxy, ketone, nitro, silyl, sulfo-oxo, sulfonyl, sulfone, sulfoxide, or thiol as described herein.

The term “cyclic group” is used herein to refer to either aryl groups, non-aryl groups (i.e., cycloalkyl, heterocycloalkyl, cycloalkenyl, and heterocycloalkenyl groups), or both. Cyclic groups have one or more ring systems that can be substituted or unsubstituted. A cyclic group can contain one or more aryl groups, one or more non-aryl groups, or one or more aryl groups and one or more non-aryl groups.

The term “aldehyde” as used herein is represented by the formula —C(O)H. Throughout this specification “C(O)” is a short hand notation for C═O.

The terms “amine” or “amino” as used herein are represented by the formula NA¹A²A³, where A¹, A², and A³ can be, independently, hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “carboxylic acid” as used herein is represented by the formula —C(O)OH. A “carboxylate” as used herein is represented by the formula —C(O)O⁻.

The term “ester” as used herein is represented by the formula —OC(O)A¹ or —C(O)OA¹, where A¹ can be an alkyl, halogenated alkyl, alkenyl, alkynyl, aryl, heteroaryl, cycloalkyl, cycloalkenyl, heterocycloalkyl, or heterocycloalkenyl group described above.

The term “ether” as used herein is represented by the formula A¹OA², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “ketone” as used herein is represented by the formula A¹C(O)A², where A¹ and A² can be, independently, an alkyl, halogenated alkyl, alkenyl, alkynyl, aryl, heteroaryl, cycloalkyl, cycloalkenyl, heterocycloalkyl, or heterocycloalkenyl group described above.

The term “halide” as used herein refers to the halogens fluorine, chlorine, bromine, and iodine.

The term “hydroxyl” as used herein is represented by the formula —OH.

The term “nitro” as used herein is represented by the formula —NO₂.

The term “cyano” as used herein is represented by the formula —CN The term “azido” as used herein is represted by the formula —N₃.

The term “sulfonyl” is used herein to refer to the sulfo-oxo group represented by the formula —S(O)₂A¹, where A¹ can be hydrogen, an alkyl, halogenated alkyl, alkenyl, alkynyl, aryl, heteroaryl, cycloalkyl, cycloalkenyl, heterocycloalkyl, or heterocycloalkenyl group described above.

The term “sulfonylamino” or “sulfonamide” as used herein is represented by the formula —S(O)₂NH₂.

The term “thiol” as used herein is represented by the formula —SH.

The term “bridging heterocylic compound” is a compound that (1) has two or more rings that share three or more atoms, (2) one or more bridges having at least one atom, and at least one heteroatom (e.g., O, S, or N). For example, 1,4-diazabicyclo[2.2.2]octane (DABCO) has two rings with a bridge composed of two carbon atoms and two heteroatoms (nitrogen). Hexamethylenetetramine (HMTA) has three rings with two bridges each composed of a carbon atom and four heteroatoms (nitrogen). The bridging heterocylic compound can be unsubstituted or substituted with one or more of the following groups: halogen, alkyl, cycloalkyl, aryl, or heteroaryl, each of with the exception of halogen may be optionally substituted with at least one substituent selected from cyano, halo, hydroxyl, nitro, mesityl, substituted mesityl, alkyl, cycloalkyl, alkoxy, phenyl, alkylphenyl, heterocyclyl, or alkylheterocyclyl.

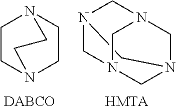

It is to be understood that the compounds provided herein may contain chiral centers. Such chiral centers may be of either the (R—) or (S—) configuration. The compounds provided herein may either be enantiomerically pure, or be diastereomeric or enantiomeric mixtures. It is to be understood that the chiral centers of the compounds provided herein may undergo epimerization in vivo. As such, one of skill in the art will recognize that administration of a compound in its (R—) form is equivalent, for compounds that undergo epimerization in vivo, to administration of the compound in its (S—) form.

As used herein, substantially pure means sufficiently homogeneous to appear free of readily detectable impurities as determined by standard methods of analysis, such as thin layer chromatography (TLC), nuclear magnetic resonance (NMR), gel electrophoresis, high performance liquid chromatography (HPLC) and mass spectrometry (MS), gas-chromatography mass spectrometry (GC-MS), and similar, used by those of skill in the art to assess such purity, or sufficiently pure such that further purification would not detectably alter the physical and chemical properties, such as enzymatic and biological activities, of the substance. Both traditional and modern methods for purification of the compounds to produce substantially chemically pure compounds are known to those of skill in the art. A substantially chemically pure compound may, however, be a mixture of stereoisomers.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

Reference will now be made in details to specific aspects of the disclosed materials, compounds, compositions, articles, and methods, examples of which are illustrated in the accompanying Examples and Figures.

### Frustrated Lewis Pair Impregnated Materials

### Frustrated Lewis Pairs

The compositions described herein include a frustrated Lewis pair (FLP). A frustrated Lewis pair is a compound or a mixture of compounds containing a Lewis acid and a Lewis base that, because of steric hindrance or geometric constraints, cannot combine to form a classical adduct. A “Lewis acid” as used herein refers to an electron pair acceptor. A “Lewis base” refers to an electron pair donor. In some embodiments, the Lewis acid and Lewis base are separate molecules forming the frustrated pair.

Any suitable Lewis acid known in the art that can be used as a component of a frustrated Lewis pair is encompassed by the present disclosure.

In one embodiment, the Lewis acid is of a compound of Formula I:

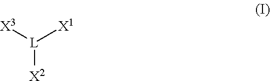

wherein:
L is a group 13 element capable of behaving as a Lewis acid, for example B or Al;
X¹ and X² are each independently selected from alkyl, cycloalkyl, aryl, or heteroaryl, each of which may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl; and
X³ is selected from hydrogen, halogen, alkyl, cycloalkyl, aryl, or heteroaryl, each of which except for hydrogen and halogen may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl.

In one embodiment of Formula I, L is selected from boron or aluminum. In one embodiment of Formula I, L is selected from aluminum.

In one embodiment of Formula I, X¹ and X² are each independently selected from C₁₋₁₂ alkyl, heteroaryl, naphthyl, mesityl, and phenyl, each of which is substituted with at least one group selected from cyano, halo, nitro, C₁₋₆ alkyl, or C₁₋₆ alkoxy.

In one embodiment of Formula I, X¹ and X² are each independently selected from phenyl or heteroaryl substituted by at least one of cyano, halo, nitro, or C₁₋₆ alkoxy. In one embodiment of Formula I, X¹ and X² are each independently phenyl or heteroaryl substituted by three to five halo, cyano, or nitro groups.

In one embodiment of Formula I, X¹ and X² are each independently phenyl or pyrrolyl substituted with at least one group selected from cyano, halo, nitro, C₁₋₆ alkyl or C₁₋₆ alkoxy.

In one embodiment of Formula I, X¹ and X² are each independently a mesityl group selected form (2,6-dimethyl)phenyl, (3,5-dimethyl)phenyl, and (3,5-bistrifluoromethyl)phenyl.

In one embodiment, the Lewis acid is a compound of Formula II:

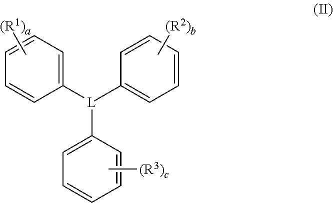

wherein:
L is as defined herein;
R¹ R², and R³ are independently selected at each occurrence from amino, cyano, halo, hydroxy, nitro, phenyl, C₁₋₆ alkyl, C₃₋₆ cycloalkyl, C₁₋₆ fluoroalkyl, C₁₋₆ chloroalkyl, C₁₋₆ alkoxy, and C₁₋₆ alkylphenyl; and
a, b, and c are independently selected from 0, 1, 2, 3, 4, or 5.

In one embodiment of Formula II, R¹, R², and R³ are each independently selected from cyano, halo, nitro, C₁₋₄ alkyl, C₁₋₄ fluoroalkyl, C₁₋₄ chloroalkyl, C₁₋₄ alkoxy and phenyl. In another embodiment of Formula II, R¹, R², and R³ are each independently selected from cyano, halo, nitro, C₁₋₄ alkyl, and C₁₋₄ alkoxy.

In another embodiment of Formula II, R¹, R², and R³ are each independently selected from chloro, fluoro, or cyano. In another embodiment of Formula II, R¹, R², and R³ are each independently selected from chloro and fluoro.

In another embodiment of Formula II, R¹, R², and R³ are each independently selected from a C₁₋₂ alkylphenyl, for example 2,6-dimethylphenyl, 3,5-dimethylphenyl, or 3,5-bistrifluoromethylphenyl.

In one embodiment of Formula II, each of a, b, or c are independently 3, 4, or 5. In another embodiment of Formula II, each of a, b, or c are 5.

In another embodiment, the Lewis Acid is a compound of Formula III:

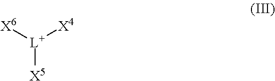

wherein:
L⁺ is a group 13 ion capable of behaving as a Lewis acid, for example B⁺ or Al⁺;
X⁴ and X⁵ are each independently selected from alkyl, cycloalkyl, haloalkyl, aryl, or heteroaryl, wherein each X⁴ and X⁵ may be optionally substituted with at least one substituent selected from cyano, halo, hydroxyl, nitro, optionally substituted mesityl, C₁₋₆ alkyl, C₃₋₆ cycloalkyl, C₁₋₆ alkoxy, phenyl, C₁₋₆ alkylphenyl, heterocyclyl, C₁₋₆ alkylheterocyclyl, SO₂aryl, and SO₂alkyl; or
X⁴ and X⁵ are brought together with the atom to which they are attached to form a four to ten atom saturated or unsaturated monocyclic or bicyclic ring which may optionally include one or two additional heteroatoms selected from nitrogen, oxygen, and sulfur and which may be optionally substituted with one or more substituents selected from amino, cyano, halo, nitro, trifluoromethyl, C₁₋₆ alkyl, C₁₋₆ alkoxy, —S(C₁₋₆ alkyl), —S(O)(C₁₋₆ alkyl), or —S(O)₂(C₁₋₆ alkyl); and
X⁶ is an electron pair donor ligand coordinated to L⁺.

In one embodiment of Formula III, L⁺ is a boron or aluminum cation. In another embodiment of Formula III, L⁺ is a boron cation.

In one embodiment of Formula III, X⁴ and X⁵ are each independently selected from fluoroalkyl, chloroalkyl, C₁₋₆ alkyl, aryl, mesityl, or substituted mesityl. In one embodiment of Formula III, X⁴ and X⁵ are each independently selected from pentafluorophenyl, pentachlorophenyl, 2,6-dimethylphenyl, 3,5-dimethylphenyl, or 3,5-bistrifluoromethylphenyl. In one embodiment of Formula III, X⁴ and X⁵ are each independently selected from 2,6-dimethylphenyl, 3,5-dimethylphenyl, or 3,5-bistrifluoromethylphenyl.

In one embodiment of Formula III, X⁴ and X⁵ are identical.

In another embodiment of Formula III, X⁴ and X⁵ are brought together with the atom to which they are attached to form an eight, nine, or ten atom saturated monocyclic or bicyclic ring. In another embodiment of Formula III, X⁴ and X⁵ are brought together with the atom to which they are attached to form a 9-borobicyclo(3.3.1)nonyl ring.

In one embodiment of Formula III, X⁶ is a strong Lewis base. In one embodiment of Formula III, X⁶ is a phosphine or carbene. In one embodiment of Formula III, X⁶ is an N-heterocyclic carbene.

Examples of suitable Lewis acids for use in the frustrated Lewis pairs include, but are not limited to, B(C₆F₃)₃, B(C₆Cl₅)₃, B(C₆F₅)(C₆Cl₅)₂, B(C₆F₅)₂(C₆Cl₅), Al(C₆F₅)₃, B(C₆F₄H)₃, BCl(C₆F₅)₂, [(ⁱPr₂—NHC)(B(2,6-(CH₃)₂C₆H₃)₂)], [(ⁱPr₂—NHC)(B(3,5-(CH₃)₂C₆H₃)₂)]⁺, or [(ⁱPr₂—NHC)(B(3,5-(CF₃)₂C₆H₃)₂)]⁺.

Additional representative examples of Lewis acids for use in the present disclosure include, but are not limited to:

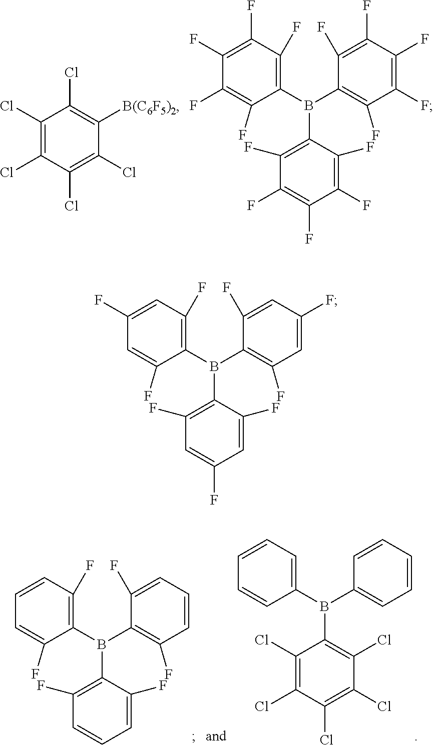

Additional examples of Lewis acids for use in the present include, but are not limited to:

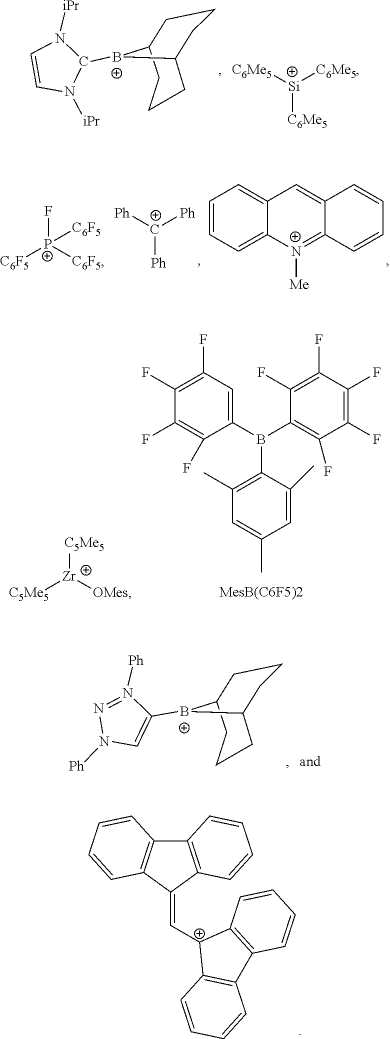

Any suitable Lewis base that can form a component of a frustrated Lewis pair is encompassed by the present disclosure, for example, sterically bulky ethers, alcohols, phosphines, or amines such as tri-tert-butylphosphine. Lewis bases for use in the present disclosure also include oxygen containing Lewis base solvents such as water, alcohols, and ethers. However, it should be understood that any molecule, complex, ion or fragment that can act as an electron pair donor and react with a Lewis acid to form a Lewis adduct can be considered a suitable Lewis base. Typically, the pKₐ of the conjugate acid of the Lewis base is about 7, but no less than about −10, in water.

In some embodiments, the Lewis base is selected from a nitrogen-containing compound, a phosphorous containing compound; an oxygen containing compound; or a sulfur containing compound.

In some embodiments, the Lewis base is a heteroaryl compound containing at least one nitrogen atom. In one embodiment, the Lewis base is a compound of Formula IV:

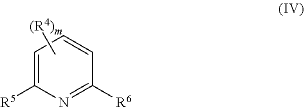

wherein:
R⁴, R⁵, and R⁶ are each independently selected at each occurrence from amino, cyano, halo, nitro, trifluoromethyl, C₁₋₆ alkyl, aryl, C₁₋₆ alkoxy, —S(C₁₋₆ alkyl), —S(O)(C₁₋₆ alkyl), and —S(O)₂(C₁₋₆ alkyl); and
m is 0, 1, 2, or 3.

In one embodiment of Formula IV, m is 0.

In one embodiment of Formula IV, R⁵ and R⁶ are independently selected from C₁₋₆ alkyl. In one embodiment of Formula IV, R⁵ and R⁶ are independently selected from methyl, ethyl, or tert-butyl. In one embodiment of Formula IV, R⁵ and R⁶ are each phenyl.

In one embodiment, the Lewis base is 2,6-dimethylpyridine or 2,6-di-tert-butylpyridine.

In some embodiments, the Lewis base is an amino compound. In one embodiment, the Lewis base is a compound of Formula V:

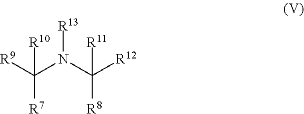

wherein:
R⁷ and R⁸ are each independently optionally substituted C₁₋₆ alkyl; or
R⁷ and R⁸ may be brought together with the atoms to which they are attached to form a five, six, or seven membered heterocyclic ring which may include one or two additional heteroatoms selected from nitrogen, oxygen or sulfur and which may be optionally substituted with one or more substituent groups selected from amino, cyano, halo, nitro, trifluoromethyl, C₁₋₆ alkyl, C₁₋₆ alkoxy, —S(C₁₋₆ alkyl), —S(O)(C₁₋₆ alkyl), or —S(O)₂(C₁₋₆ alkyl);
R⁹, R¹⁰, R¹¹, and R¹² are selected from hydrogen, alkyl, cycloalkyl, aryl, or heteroaryl, each which may be optionally substituted with one or more substituents selected form amino, cyano, halo, nitro, trifluoromethyl, C₁₋₆ alkyl, C₁₋₆ alkoxy, —S(C₁₋₆ alkyl), —S(O)(C₁₋₆ alkyl), or —S(O)₂(C₁₋₆ alkyl); and
R¹³ is hydrogen, C₁₋₃ alkyl, aryl, or heteroaryl.

In one embodiment of Formula V, R⁹, R¹⁰, R¹¹, and R¹² are each selected from C₁₋₆ alkyl. In one embodiment of Formula V, R⁹, R¹⁰, R¹¹, and R¹² are each methyl.

In one embodiment of Formula V, R⁷ and R⁸ are brought together to form a piperidine ring.

In one embodiment, R¹³ is hydrogen.

In one embodiment, the Lewis base is 2,2,6,6,-tetramethylpiperidine.

In some embodiments, the Lewis base is an N-heterocyclic carbene. In one embodiment, the Lewis base is a compound of Formula VI:

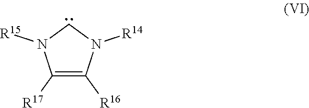

wherein:
R¹⁴ and R¹⁵ are each selected from hydrogen or C₁₋₆ alkyl; and
R¹⁶ and R¹⁷ are each selected from amino, cyano, halo, hydrogen, nitro, trifluoromethyl, C₁₋₆ alkyl, C₁₋₆ alkoxy, NH(C₁₋₆ alkyl), N(independently C₁₋₆ alkyl)₂, —S(C₁₋₆ alkyl), —S(O)(C₁₋₆ alkyl), —S(O)₂(C₁₋₆ alkyl), or phenyl.

In one embodiment of Formula VI, R¹⁴ and R¹⁵ are independently C₁₋₆ alkyl. In one embodiment of Formula VI, R¹⁴ and R¹⁵ are independently each methyl, ethyl, n-propyl, isopropyl, n-butyl, sec-butyl, or tert-butyl.

In one embodiment of Formula VI, R¹⁶ and R¹⁷ are both hydrogen.

In some embodiments, the Lewis base is a phosphorous containing compound. In one embodiment, the Lewis base is a compound of Formula VII:

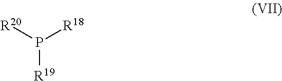

wherein R¹⁸, R¹⁹, and R²⁰ are each independently selected from alkyl, cycloalkyl, aryl, heterocyclyl, and heteroaryl, each of which may be optionally substituted with one or more substituent selected from amino, aryl, cyano, halo, heteorcyclyl, nitro, C₁₋₆ alkyl, or C₁₋₆ alkoxy.

In one embodiment of Formula VII, R¹⁸, R¹⁹, and R²⁰ are each independently selected from C₁₋₆ alkyl or phenyl, each of which is optionally substituted with amino, cyano, halo, nitro, C₁₋₆ alkyl, or C₁₋₆ alkoxy.

In one embodiment of Formula VII, R¹⁸, R¹⁹ and R²⁰ are each independently selected from C₁₋₆ alkyl or phenyl, each of which is optionally substituted with halo, C₁₋₆ alkyl, or C₁₋₆ alkoxy.

In one embodiment of Formula VII, R¹⁸, R¹⁹, and R²⁰ are each independently selected from C₁₋₆ alkyl or phenyl, each of which is optionally substituted with halo, C₁₋₆ alkyl, C₁₋₆ alkoxy, or pentafluorophenyl.

In one embodiment of Formula VII, R¹⁹ and R²⁰ are each phenyl substituted with one, two, or three methyl groups and R¹⁸ is ethyl, t-butyl, or phenyl substituted with halo or B(C₆F₅)₂.

In one embodiment of Formula VII, R¹⁸, R¹⁹, and R²⁰ are each t-butyl or phenyl substituted with one, two, or three methyl groups.

In some embodiments, the Lewis base is an oxygen containing compound. In one embodiment, the Lewis base is a compound of Formula VIII:

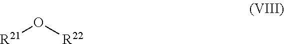

wherein:
R²¹ and R²² are independently selected from alkyl, cycloalkyl, aryl, heterocyclyl, or heteroaryl, each of which is optionally substituted with amino, aryl, cyano, halo, heterocyclyl, nitro, C₁₋₆ alkyl, or C₁₋₆ alkoxy; or
R²¹ and R²² may be brought together with the oxygen to which they are attached to form a four to seven membered saturated or unsaturated ring which may optionally include one or two additional heteroatoms selected from nitrogen, oxygen, or sulfur and which may be optionally substituted with one or more substituents selected from amino, cyano, halo, nitro, trifluoromethyl, C₁₋₆ alkyl, C₁₋₆ alkoxy, —S(C₁₋₆ alkyl), —S(O)(C₁₋₆ alkyl), and —S(O)₂(C₁₋₆ alkyl).

In one embodiment of Formula VIII, R²¹ and R²² are each C₁₋₆ alkyl.

In another embodiment of Formula VIII, R²¹ and R²² are brought together with the oxygen to which they are attached to form a five, six, or seven membered saturated or unsaturated ring which may optionally include one or two more heteroatoms selected from nitrogen, oxygen, or sulfur and which may be optionally substituted with one or more substituents selected from halo, trifluoromethyl, C₁₋₆ alkyl, or C₁₋₆ alkoxy. In one embodiment, the compound of Formula VIII is selected from N-methylmorpholine, 1,4-dioxane, tetrahydrofuran, or tetrahydropyran.

In some embodiment, the Lewis base is a sulfur containing compound. In one embodiment, the Lewis base is a compound of Formula IX:

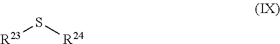

wherein:
R²³ and R²⁴ are independently selected from alkyl, cycloalkyl, aryl, heterocyclyl, or heteroaryl, each of which is optionally substituted with amino, aryl, cyano, halo, heterocyclyl, nitro, C₁₋₆ alkyl, or C₁₋₆ alkoxy; or
R²³ and R²⁴ may be brought together with the sulfur to which they are attached to form a four to seven membered saturated or unsaturated ring which may optionally include one or two additional heteroatoms selected from nitrogen, oxygen, or sulfur and which may be optionally substituted with one or more substituents selected from amino, cyano, halo, nitro, trifluoromethyl, C₁₋₆ alkyl, C₁₋₆ alkoxy, —S(C₁₋₆ alkyl), —S(O)(C₁₋₆ alkyl), and —S(O)₂(C₁₋₆ alkyl).

In one embodiment, R²³ and R²⁴ are independently C₁₋₆ alkyl.

In one embodiment, R²³ and R²⁴ are brought together with the sulfur to which they are attached to form a five, six, or seven membered saturated or unsaturated ring which may be optionally substituted with one or more substituent groups selected from halo, trifluoromethyl, C₁₋₆ alkyl, or C₁₋₆ alkoxy. In one embodiment, R²³ and R²⁴ are brought together to form an optionally substituted thiophene ring. In one embodiment, R²³ and R²⁴ are brought together to form an optionally substituted five membered saturated ring, i.e. a tetrahydrothiophene ring.

In another embodiment, the Lewis base may be an optionally substituted sulfolane or an optionally substituted dialkyl sulfoxide.

Other representative examples of Lewis bases that may be used in the present disclosure include: N-methylmorpholine; nitriles; imidazole; phosphines; ethers (including symmetric and non-symmetric dialkyl or diaryl ethers); dioxane; furans (such as tetrahydrofuran); carbonyl containing compounds (such as acetone or methylethylketone); amides (such as dimethylacetamide, diethylacetamide, or acetamide); alcohols (such as mono, bi, tri, or tert alcohols or alkyl, aryls, or phenyls); thiophenes; tetrahydrothiophenes; dimethylsulfoxide; and sulfolane.

In one embodiment, the Lewis base is a bridging heterocylic compound such as, for example, 1,4-diazabicyclo[2.2.2]octane (DABCO) and hexamethylenetetramine (HMTA). In another embodiment, the Lewis base includes:

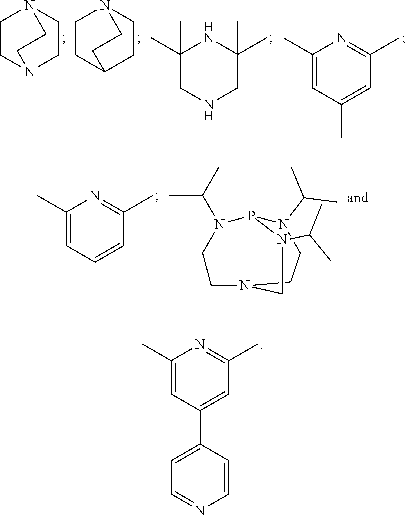

Additional representative examples of Lewis bases that may be used in the present disclosure include:

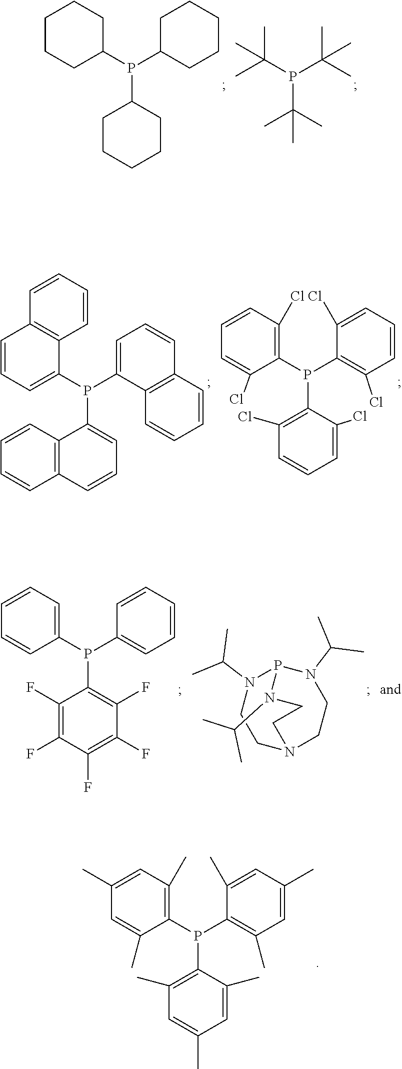

Additional representative examples of Lewis bases that may be used in the present disclosure include: diethyl ether; 1,4-dioxane; tetrahydrofuran; and tetrahydropyran.

Additional examples of Lewis bases that may be used in the present disclosure include, but are not limited to:

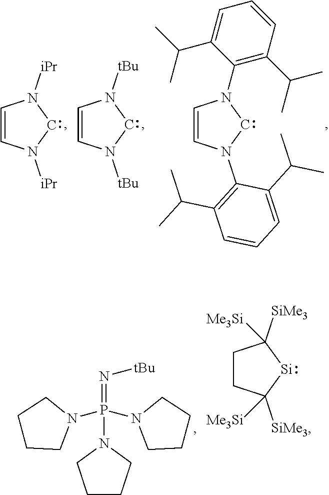

In another embodiment, the Lewis acid and Lewis base are separate moieties on the same molecule, i.e. the Lewis acid and the Lewis base are covalently linked by a divalent organic linker. The precise nature of the linker that connects the Lewis acid and Lewis base are not critical as long as the Lewis acid and Lewis base are still prevented from forming an adduct. For example, the linker may be a C₁₋₆ alkylene (e.g. methylene or ethylene) or a C₀₋₆ alkylene-phenylene (e.g. —CH₂-phenylene-).

Representative examples of frustrated Lewis pairs having a Lewis acid and Lewis base moiety in the in the same molecule include:

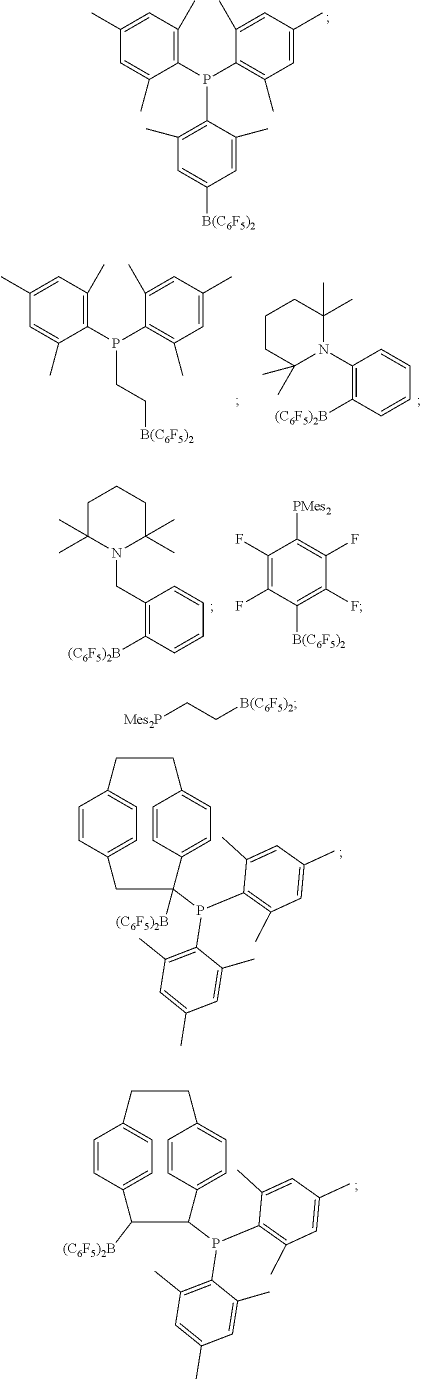

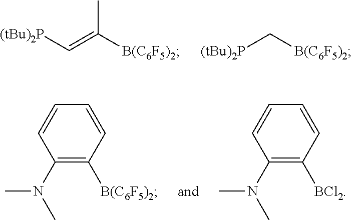

In some embodiments, the Lewis base may be incorporated, i.e. covalently bound, to an organic linker ligand as used in the synthesis of a metal-organic framework as described further below. Representative examples of such ligands include, but are not limited to:

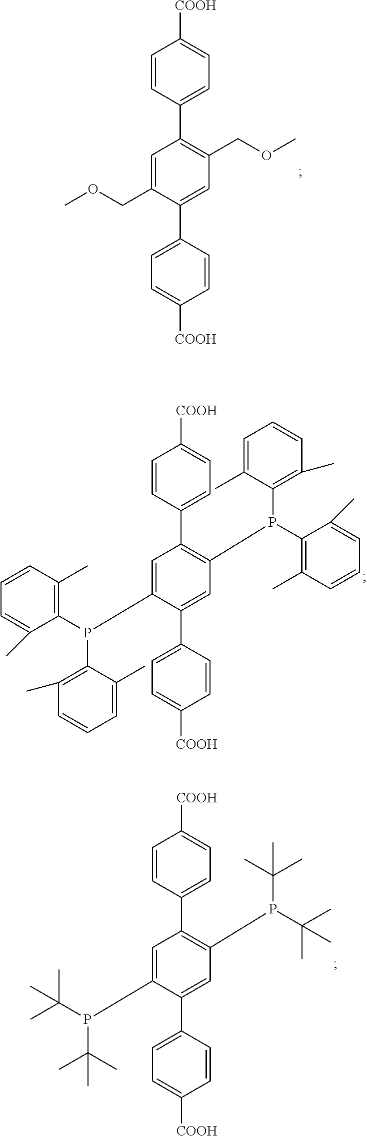

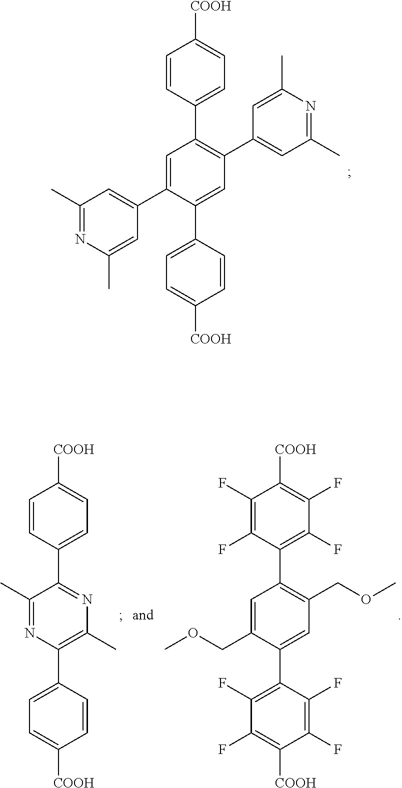

Additional examples of such ligands include, but are not limited to:

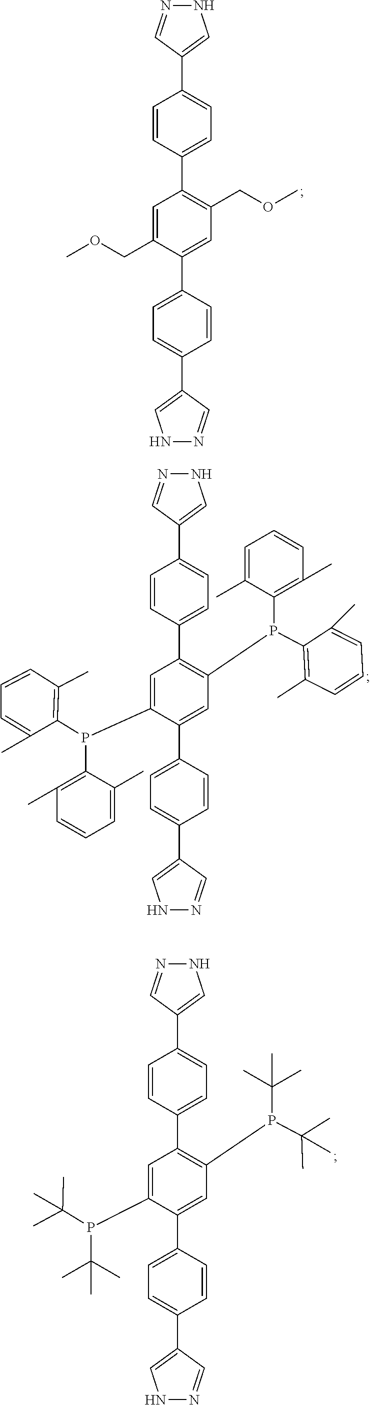

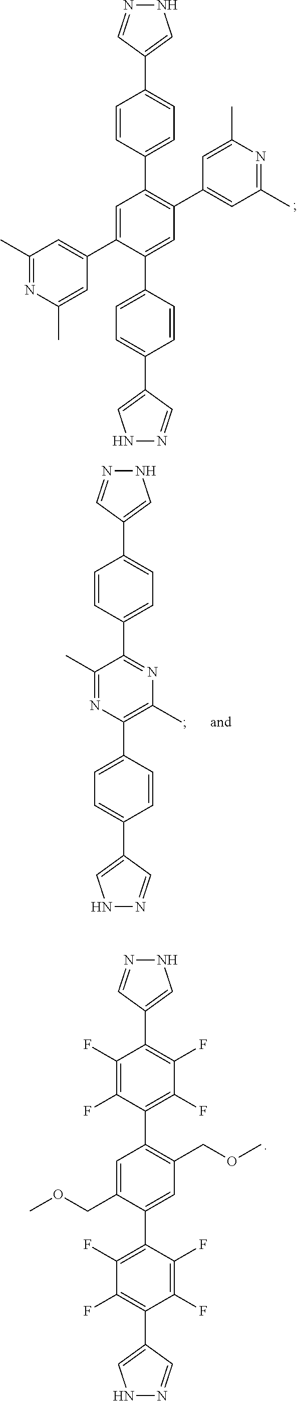

Additional examples of such ligands include, but are not limited to:

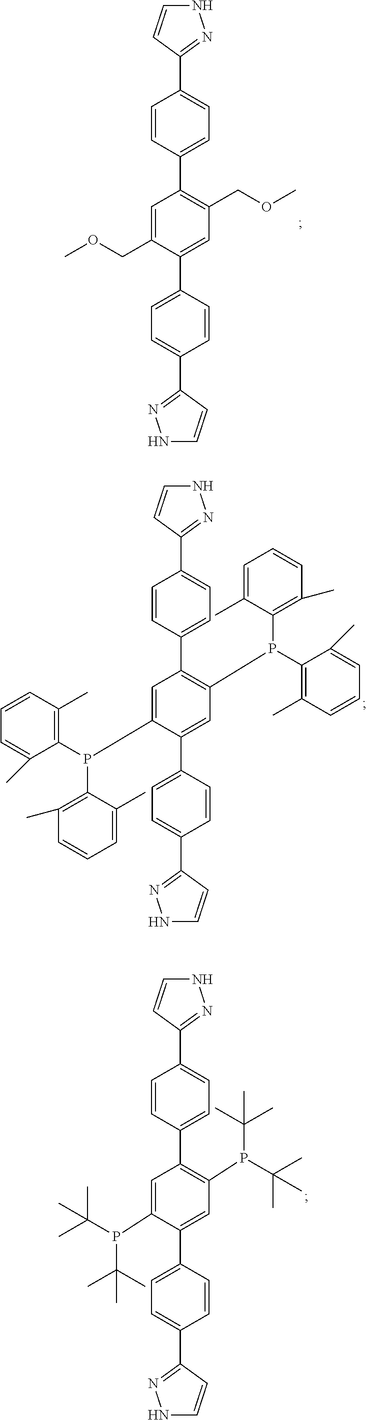

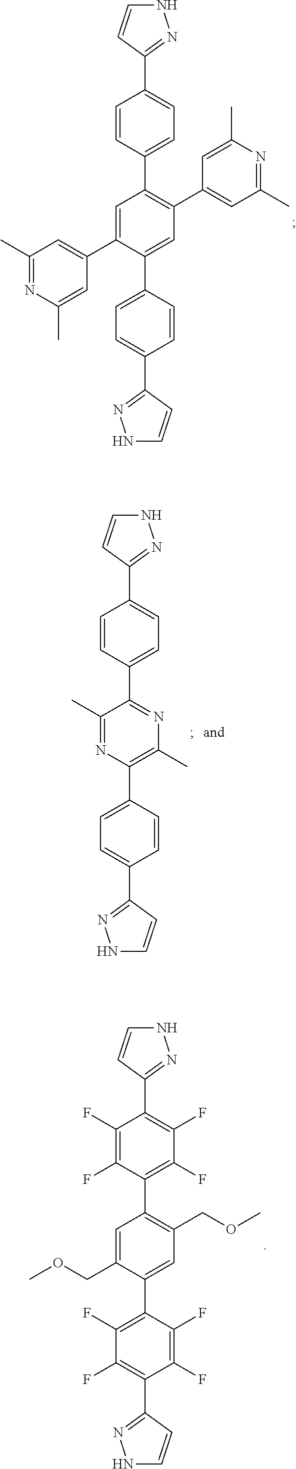

Additional examples of such ligands include, but are not limited to:

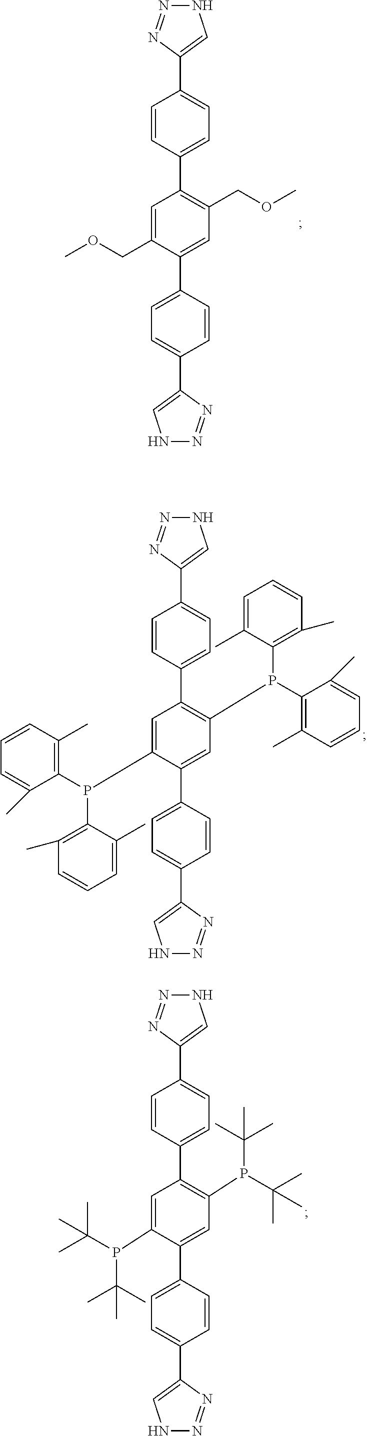

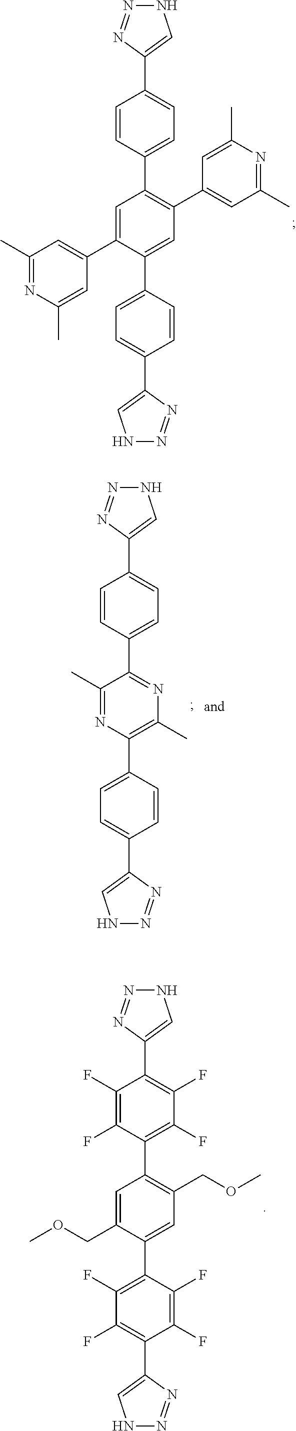

Additional examples of such ligands include, but are not limited to:

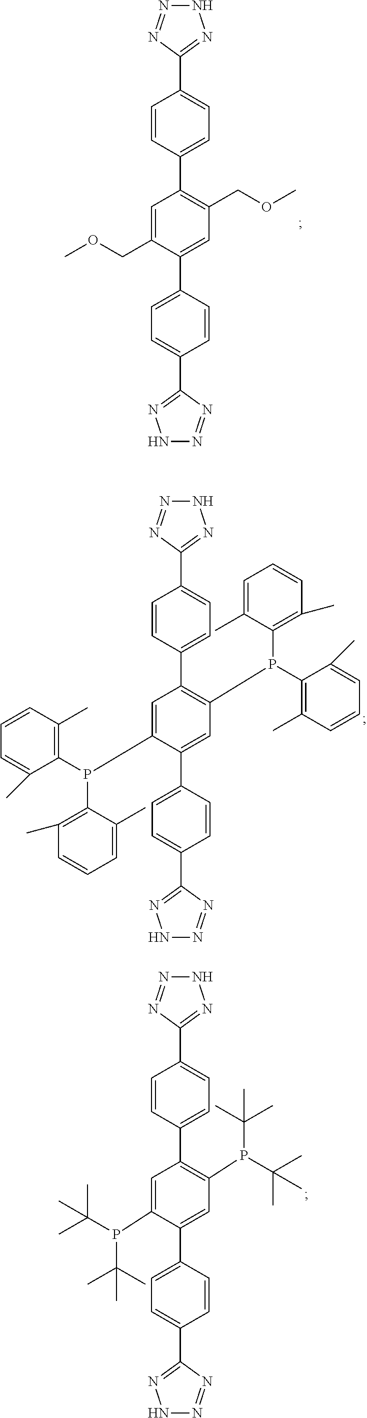

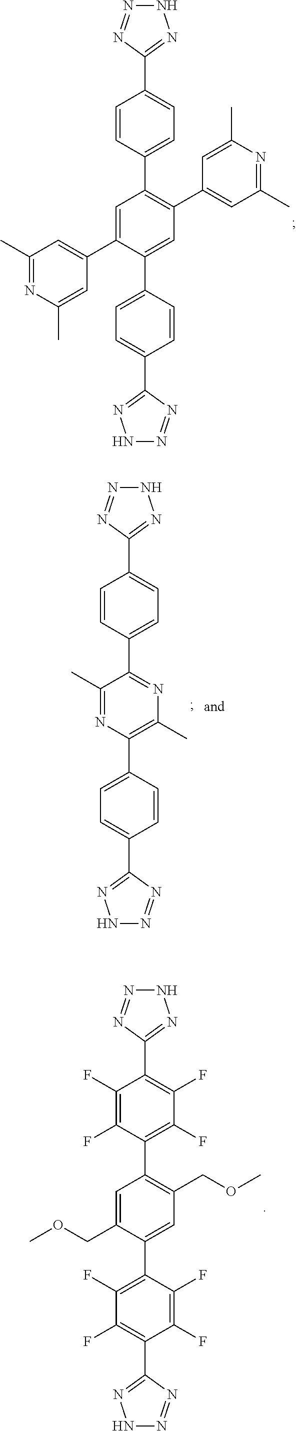

In some embodiments, the Lewis Acid, Lewis base, or frustrated Lewis pair having both a Lewis acid and Lewis base moiety in the same molecule may be chiral. Representative examples include, but are not limited to:

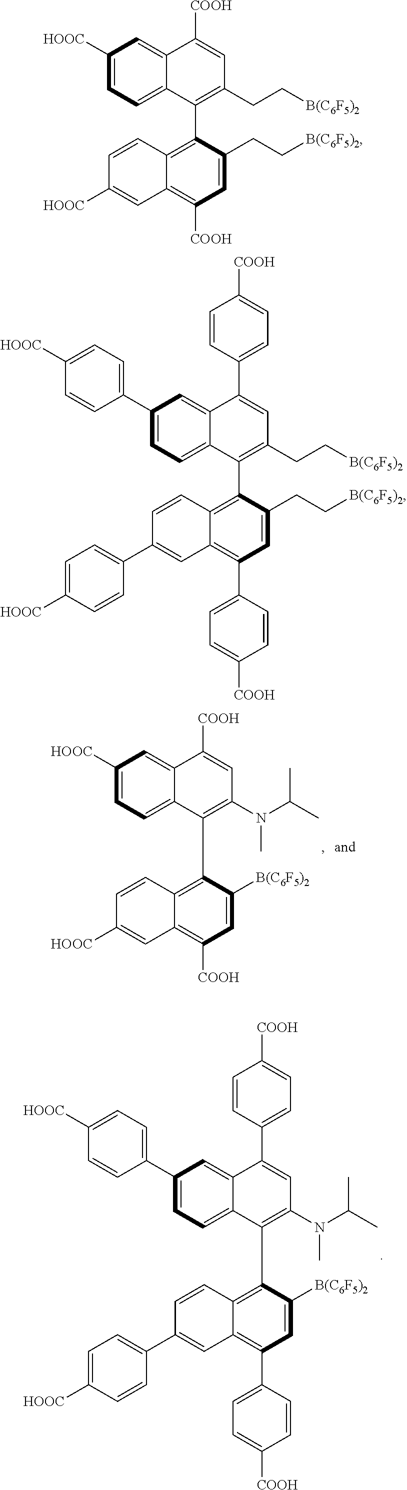

In some embodiments, the frustrated Lewis pair as used in the present disclosure is selected from a frustrated Lewis pair described in: WO 2011/045605; WO 2013/17708; WO 2016/168914; WO 2016/198892; and WO 2017/100904; each of which is incorporated herein by reference in their entirety.

In some embodiments, the frustrated Lewis pair as used in the present disclosure is selected from a frustrated Lewis pair described in: Angew. Chem. Int. Ed. 2015, 54(22):6400-6441; Trends in Chemistry 2019, 1(1):35-48; Science 2016, 354(6317):aaf7229; Chem. Soc. Rev. 2017, 46:5689-5700; and Synthesis 2018, 50(9):1783-1795; each of which is incorporated herein by reference in their entirety.

### Porous Materials

Any porous material that can be impregnated with a frustrated Lewis pair can be used in the formation of the compositions of the present disclosure. In some embodiments, the porous material may be selected from a porous metal-organic framework (MOF) or a porous organic polymer (POP).

In one embodiment, the porous material is a porous organic polymer. A “porous organic polymer” (POP) as used herein refers generally to high surface area materials formed from organic segments covalently bonded to form an extended porous structure. Porous organic polymers can include porous aromatic frameworks, porous polymer networks, and porous organic frameworks. The porous organic polymer can be crystalline, semi-crystalline, or amorphous. The porous organic polymer can have a surface greater than about 20 m²/g, 50 m²/g, 100 m²/g, 500 m²/g, or greater than about 1,000 m²/g. The porous organic polymer can have a surface are up to about 8,000 m²/g, 7,000 m²/g, 6,000 m²/g, 5,000 m²/g, or 4,000 m²/g. As used herein, the term “porous organic polymer” does not include zeolite structures or mesoporous silica structures.

In one embodiment, the porous organic polymer is a porous aromatic framework (PAF). “Porous aromatic framework” (PAF) as used herein refers to a class of ultrahigh surface area materials characterized by a rigid aromatic open-framework structure constructed by covalent bonds. Porous aromatic frameworks lack the extended conjugation found in conjugated microporous polymers. A porous aromatic framework can have a surface area from about 500 m²/g to about 7,000 m²/g.

In one embodiment, the porous organic polymer is a porous polymer network (PPN). A “porous polymer network” (PPN) or “interpenetrating polymer network” (IPN) as used interchangeable herein, refers to a class of high surface area materials containing at least two polymers, each in network form wherein at least one of the polymers is synthesized and/or crosslinked in the presence of the other. The polymer networks are physically entangled with each other and in some embodiments may also be covalently bonded. Porous polymer networks can have a surface area from about 20 m²/g to about 6,000 m²/g.

In some embodiments, the porous organic polymer is a covalent organic framework (COF) or porous organic framework (POF). A “covalent organic framework” (COF) or “porous organic framework” (POF) as used interchangeably herein, refers to a class of highly crystalline, high surface area materials formed of small organic building blocks made entirely from light elements (H, B, N, C, and O) that are known to form strong covalent bonds. Porous organic frameworks can have a surface area from about 100 m²/g to about 5,000 m²/g.

Suitable porous organic polymers can include, but are not limited to, fluoropolymers (e.g. polytetrafluoroethylene or polyvinylidene fluoride) polyolefins (e.g. polyethylene or polypropylene), polyamides, polyesters, polysulfones, poly(ethersulfone), polycarbonates, polyurethanes, and combinations thereof. Suitable porous aromatic frameworks can include, but are not limited to, cross-linked poly-tetraphenylmethane, poly-teraphenyl silane, and poly-triphenyl amine polymers.

The porous organic polymer can contain monomer units having an aryl moiety. A variety of porous organic polymers can be made with aryl moieties. For example, the porous organic polymer can contain a monomer unit containing an aryl moiety selected from the group consisting of substituted and unsubstituted benzene, naphthalene, anthracene, biphenyl, pyridine, pyrimidine, pyridazine, pyrazine, and triazine. In some embodiments, the porous organic polymer is as described in WO 2015/105871 or WO 2016/028434, each of which is incorporated herein by reference in their entirety.

### Metal-Organic Frameworks as Porous Materials

In one embodiment, the porous material is a metal-organic framework. Metal-organic frameworks are highly crystalline three-dimensional inorganic-organic hybrids constructed by assembling metal ions or small metal-containing clusters with multidentate organic ligands (e.g. carboxylates, tetrazolates, sulfonates) via coordination bonds. Metal-organic frameworks are materials in which metal to organic ligand interactions can form a porous coordination network. Metal-organic frameworks are coordination polymers with an inorganic-organic hybrid frame comprising metal ions or clusters of metal ions and organic ligands coordinated with the metal ions and/or clusters. These materials are organized in a one-, two- or three-dimensional framework in which the metal clusters are linked to one another periodically by bridging ligands and/or pillar ligands. In one embodiment, the inorganic sections can be referred to as secondary building units (SBU) and these can include the metal or metal clusters and bridging ligands. SBUs can be connected by pillar ligands (and/or hybrid pillar/bridging ligands) to form metal-organic frameworks. Typically these materials have a crystal structure. In one embodiment, the nanospace of the metal-organic framework can have a diameter of about 2 to 50 nm. In another embodiment, the metal-organic framework has a pore or window size of about 2 nm to 50 nm.

The term “metal” as used within the scope of the present disclosure can refer to metal, metal ions, and/or clusters of metal or metal ions, that are able to form a metal-organic, porous framework material. In an embodiment, the metal can include metals corresponding to the Ia, IIa, IIIa, IVa to VIIIa and Ib to VIIIb groups of the periodic table of elements. In an embodiment, the metal (or metal ion) can include: Mg, Ca, Sr, Ba, Sc, Y, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Hg, Al, Ga, In, Tl, Si, Ge, Sn, Pb, As, Sb, and Bi, as well as di-metals. In an embodiment, the metal ion can have a 1⁺, 2⁺, 3⁺, 4⁺, 5⁺, 6⁺, 7⁺, or 8 charge. In one embodiment, the metal is Ag. In one embodiment, the metal is Al. In one embodiment, the metal is Be. In one embodiment, the metal is Ca. In one embodiment, the metal is Cd. In one embodiment, the metal is Ce. In one embodiment, the metal is Co. In one embodiment, the metal is Cr. In one embodiment, the metal is Cu. In one embodiment, the metal is Dy. In one embodiment, the metal is Er. In one embodiment, the metal is Eu. In one embodiment, the metal is Fe. In one embodiment, the metal is Ga. In one embodiment, the metal is Gd. In one embodiment, the metal is Ho. In one embodiment, the metal is In. In one embodiment, the metal is Li. In one embodiment, the metal is Mg. In one embodiment, the metal is Mn. In one embodiment, the metal is Mo. In one embodiment, the metal is Nd. In one embodiment, the metal is Ni. In one embodiment, the metal is Sc. In one embodiment, the metal is Sm. In one embodiment, the metal is Sr. In one embodiment, the metal is Tb. In one embodiment, the metal is Tm. In one embodiment, the metal is V. In one embodiment, the metal is W. In one embodiment, the metal is Y. In one embodiment, the metal is Yb. In one embodiment, the metal is Zn. In one embodiment, the metal is Zr.

In some embodiments, the organic linker ligands can include bridging ligands and pillar ligands. In some embodiments, the bridging ligands (e.g. coordinating to the metal or metal cluster) and/or the pillar ligands (e.g. linking layers of the metal-organic framework) can include one or more functional groups that can coordinate with the metal(s) and/or link metal containing groups (e.g. some ligands can act as bridging and pillar ligands). The ligand may be any ligand suitable for forming a metal-organic framework. Typically, the ligand is at least a bidentate organic compound. In some embodiments, the ligand is selected from the group consisting of organic bidentate, tridentate, tetradentate, or more generally, polydentate compounds that are capable of coordinating to metal ions. The term “at least bidentate organic compound” as use within the scope of the present disclosure typically refers to an organic compound comprising at least one functional group which is able to form at least two bonds, preferably two coordinate bonds, to a given metal ion and/or to form one coordinative bond each to two or more metal ions.

Examples of functional groups to be mentioned, via which the coordinative bonds can be formed, include the following functional groups in particular: —CO₂H, —CS₂H, —NO₂, —B(OH)₂, —SO₃H, —Si(OH)₃, —Ge(OH)₃, —Sn(OH)₃, Si(SH)₄, —Ge(SH)₄, —Sn(SH)₃, —PO₃H, AsO₃H, AsO₄H, —P(SH)₃, As(SH)₃, CH(alkylene- or arylene-SH)₂, C(alkylene or arylene-SH)₂, —CH(alkylene or arylene-SNH₂)₂, —C(alkylene or arylene-NH₂)₃, —CH(alkylene or arylene-OH)₂, —C(alkylene or arylene-OH)₃, —CH(alkylene or arylene-CN)₂, and C(alkylene or arylene-CN)₃.

The at least two functional groups can be in principal bound to any suitable organic compound, as long as there is the assurance that the organic compound having these functional groups is capable of forming the coordinative bond and of producing the framework material.

The organic compounds comprising the at least two functional groups are typically derived from a saturated or unsaturated aliphatic compound (e.g. alkane, alkene, and the like having 2 to 20 carbons) or an aromatic compound (e.g. having 4 to 8 carbons per ring) or a compound which is both aliphatic and aromatic.

In one embodiment, the organic compound can include a polycarboxylate ligand (e.g. dicarboxylate ligand, tricarboxylate ligand, or tetra/hexa/octacarboxylate ligand), a polypyridyl ligand (e.g. dipyridyl ligand, tripyridyl ligand, or tetra/hexa/octapyridyl ligand), a polycyano ligand (e.g. dicyano ligand, tricyano ligand, or tetra/hexa/octacyano ligand), a polyphosphonate ligand (e.g. diphosphonate ligand, triphosphonate ligand, or tetra/hexa/octaphosphonate ligand), a polyhydroxyl ligand (e.g. dihydroxyl ligand, trihydroxyl ligand, or tetra/hexa/octahydroxyl ligand), a polysulfonate ligand (e.g. disulfonate ligand, trisulfonate ligand, or tetra/hexa/octasulfonate ligand), a polyimidazolate ligand (e.g. diimidazolate ligand, triimidazolate ligand, or tetra/hexa/octaimidazolate ligand), a polytriazolate (both 1,2,3 and 1,2,4 triazolate) ligand (e.g. ditriazolate ligand, tritriazolate ligand, or tetra/hexa/octatriazolate ligand), polytetrazolate ligand (e.g. ditetrazolate ligand, tritetrazolate ligand, or tetra/hexa/octatetrazolate ligand), polypyrazolate ligand (e.g. dipyrazolate ligand, tripyrazolate ligand, or tetra/hexa/octapyrazolate ligand), and mixtures or combinations thereof.

Representative examples of carboxylic acids that may be used as organic ligands in the present disclosure include, but are not limited to:
dicarboxylic acids such as 1,4-butanedicarboxylic acid; 4-oxopyran-2,6-dicarboxylic acid; 1,6-hexanedicarboxylic acid; decanedicarboxylic acid; 1,8-heptadecanedicarboxylic acid; 1,9-heptadecanedicarboxylic acid; heptadecanedicarboxylic acid; acetylenedicarboxylic acid; 1,2-benzenedicarboxylic acid; 2,3-pyridinedicarboxylic acid; pyridine-2,3-dicarboxylic acid; 1,3-butadiene-1,4-dicarboxylic acid; 1,4-benzenedicarboxylic acid; p-benzenedicarboxylic acid; imidazole-2,4-dicarboxylic acid; 2-methylquinoline-3,4-dicarboxylic acid; quinoline-2,4-dicarboxylic acid; quinoxaline-2,3-dicarboxylic acid; 6-chloroquinoxaline-2,3-dicarboxylic acid; 4,4′-diaminophenylmethane-3,3′-dicarboxylic acid; quinoline-3,4-dicarboxylic acid; 7-chloro-4-hydroxyquinoline-2,8-dicarboxylic acid; diimidedicarboxylic acid; pyridine-2,6-dicarboxylic acid; 2-methylimidazole-4,5-dicarboxylic acid; thiophene-3,4-dicarboxylic acid; 2-isopropylimidazole-4,5-dicarboxylic acid; tetrahydropyran-4,4-dicarboxylic acid; perylene-3,9-dicarboxylic acid; perylenedicarboxylic acid; Pluriol E 200-dicarboxylic acid; 3,6-dioxaoctanedicarboxylic acid; 3,5-cyclohexadiene-1,2-dicarboxylic acid; octanedicarboxylic acid; pentane-3,3-carboxylic acid; 4,4′-diamino-1,l′-diphenyl-3,3′-dicarboxylic acid; 4,4′-diaminodiphenyl-3,3′-dicarboxylic acid; benzidine-3,3′-dicarboxylic acid; 1,4-bis-(phenylamino)benzene-2,5-dicarboxylic acid; 1,l′-dinaphthyl-8,8′-dicarboxylic acid; 7-chloro-8-methylquinoline-2,3-dicarboxylic acid; 1-anilinoanthraquinone-2,4′-dicarboxylic acid; polytetrahydrofuran-250-dicarboxylic acid; 1,4-bis(carboxymethyl)piperazine-2,3-dicarboxylic acid; 7-chloroquinoline-3,8-dicarboxylic acid; 1-(4-carboxy)phenyl-3-(4-chloro)-phenylpyrazoline-4,5-dicarboxylic acid; 1,4,5,6,7,7,-hexachloro-5-norbornene-2,3-dicarboxylic acid; phenylindanedicarboxylic acid; 1,3-dibenzyl-2-oxoimidazolidine-4,5-dicarboxylic acid; 1,4-cyclohexanedicarboxylic acid; naphthalene-1,8-dicarboxylic acid; 2-benzoylbenzene-1,3-dicarboxylic acid; 1,3-dibenzyl-2-oxoimidazolidine-4,5-cisdicarboxylic acid; 2,2′-biquinoline-4,4′-dicarboxylic acid; pyridine-3,4-dicarboxylic acid; 3,6,9-trioxaundecanedicarboxylic acid; o-hydroxybenzophenonedicarboxylic acid; Plunol E 300-dicarboxylic acid; Plunol E 400-dicarboxylic acid; Pluriol E 600-dicarboxylic acid; pyrazole-3,4-dicarboxylic acid; 2,3-pyrazinedicarboxylic acid; 5,6-dimethyl-2,3-pyrazinedicarboxylic acid; 4,4′-diaminodiphenyletherdiimidedicarboxylic acid; 4,4′-diaminodiphenylmethanediimidedicarboxylic acid; 4,4′ diaminodiphenylsulfonediimidedicarboxylic acid; 2,6-naphthalenedicarboxylic acid; 1,3-adamantanedicarboxylic acid; 1,8-naphthalenedicarboxylic acid; 2,3-naphthalenedicarboxylic acid; 8-methoxy-2,3-naphthalenedicarboxylic acid; 8-nitro-2,3-naphthalenedicarboxylic acid; 8-sulfo-2,3-naphthalenedicarboxylic acid; anthracene-2,3-dicarboxylic acid; 2′-3′-diphenyl-p-diphenylether-4,4′-dicarboxylic acid; imidazole-4,5-terphenyl-4,4″-dicarboxylic acid; dicarboxylic acid; 4(1H)-oxothiochromene-2,8-dicarboxylic acid; 5-t-butyl-1,3-benzenedicarboxylic acid; 7,8-quinolinedicarboxylic acid; 4,5-imidazoledicarboxylic acid; 4-cyclohexene-1,2-dicarboxylic acid; hexatriacontanedicarboxylic acid; tetradecanedicarboxylic acid; 1,7-heptanedicarboxylic acid; 5-hydroxy-1,3-benzenedicarboxylic acid; pyrazine-2,3-dicarboxylic acid; furan-2,5-dicarboxylic acid; 1-nonene-6,9-dicarboxylic acid; eicosenedicarboxylic acid, 4,4′-dihydroxydiphenylmethane-3,3′-dicarboxylic acid; 1-amino-4-methyl-9,10-dioxo-9,10-dihydroanthracene-2,3-dicarboxylic acid; 2,5-pyridinedicarboxylic acid; cyclohexene-2,3-dicarboxylic acid; 2,9-dichlorofluorubin-4,11-dicarboxylic acid; 7-chloro-3-methylquinoline-6,8-dicarboxylic acid; 2,4-dichlorobenzophenone-2′,5′-dicarboxylic acid; 1,3-benzenedicarboxylic acid; 2,6-pyridinedicarboxylic acid; 1-methylpyrrole-3,4-dicarboxylic acid; 1-benzyl-1H-pyrrole-3,4-dicarboxylic acid; anthraquinone-1,5-dicarboxylic acid; 3,5-pyrazoledicarboxylic acid; 2-nitrobenzene-1,4-dicarboxylic acid; heptane-1,7-dicarboxylic acid; cyclobutane-1,1-dicarboxylic acid; 1,14-tetradecanedicarboxylic acid; 5,6-dehydronorbornane-2,3-dicarboxylic acid; or 5-ethyl-2,3-pyridinedicarboxylic acid;
tricarboxylic acids such as 2-hydroxy-1,2,3-propanetricarboxylic acid; 7-chloro-2,3,8-quinolinetricarboxylic acid; 1,2,4-benzenetricarboxylic acid; 1,2,4-butanetricarboxylic acid; 2-phosphono-1,2,4-butanetricarboxylic acid; 1,3,5-benzenetricarboxylic acid; 1-hydroxy-1,2,3-propanetricarboxylic acid; 4,5-dihydro-4,5-dioxo-1H-pyrrolo[2,3-F]quinoline-2,7,9-tricarboxylic acid; 5-acetyl-3-amino-6-methylbenzene-1,2,4-tricarboxylic acid; 3-amino-5-benzoyl-6-methylbenzene-1,2,4-tricarboxylic acid; 1,2,3-propanetricarboxylic acid; or aurinetricarboxylic acid;
or tetracarboxylic acids such as 1,1-dioxide-perylo[1,12-BCD]thiophene-3,4,9,10-tetracarboxylic acid; perylenetetracarboxylic acids such as perylene-3,4,9,10-tetracarboxylic acid or perylene-1,12-sulfone-3,4,9,10-tetracarboxylic acid; butanetetracarboxylic acids such as 1,2,3,4-butanetetracarboxylic acid or meso-1,2,3,4-butanetetracarboxylic acid; decane-2,4,6,8-tetracarboxylic acid; 1,4,7,10,13,16-hexaoxacyclooctadecane-2,3,11,12-tetracarboxylic acid; 1,2,4,5-benzenetetracarboxylic acid; 1,2,11,12-dodecanetetracarboxylic acid; 1,2,5,6-hexanetetracarboxylic acid; 1,2,7,8-octanetetracarboxylic acid; 1,4,5,8-naphthalenetetracarboxylic acid; 1,2,9,10-decanetetracarboxylic acid; benzophenonetetracarboxylic acid; 3,3′,4,4′-benzophenonetetracarboxylic acid; tetrahydrofurantetracarboxylic acid; or cyclopentanetetracarboxylic acids such as cyclopentane-1,2,3,4-tetracarboxylic acid.

In one embodiment, the ligand is selected from: 1,2,4,5-tetrakis(4-carboxyphenyl)benzene; 1,3,5-tris(4′-carboxy[1,1′-biphenyl]-4-yl)benzene; 1,3,5-tris(4-carboxyphenyl)benzene; 2,5-dihydroxyterephthalic acid; 2,6-naphthalenedicarboxylic acid; 2-hydroxyterephthalic acid; 2-methylimidazole; 3,3′,5,5′-tetracarboxydiphenylmethane; 4,4′,4″-s-triazine-2,4,6-triyl-tribenzoic acid; 9,10-anthracenedicarboxylic acid; biphenyl-3,3′,5,5′-tetracarboxylic acid; biphenyl-3,4′,5-tricarboxylic acid; imidazole, terephthalic acid; trimesic acid; [1,1′:4′,1″]terphenyl-3,3′,5,5′-tetracarboxylic acid; or combinations thereof.

Representative examples of metal-organic frameworks as may be used in the present disclosure include, but are not limited to: Al(OH)(BDC), Al-MIL-53-NH₂, AL-MIL-53-X, BCF-1, BCF-2, BIF-10, BIF-11, BIF-12, BZ1, CAU-1, CAU-5, CAUMOF-8, CdIF-1, Ce-BTC, Ce-MDIP1, Ce-MDIP2, Co(Im)₄, CO/DOBDC, Co₃(BHTC)₂, CO₃(BTC)₂, COP-1(Zn), CPF-1, CPM-18(In), CPM-18(Nd), CPM-18(Sm), CPM-20, CPM-20(Co), CPM-24(Co), CPM-24, CPO-1(Cd), CPO-20, CPO-21, CPO-22, CPO-26(Mg), CPO-26(Mn), CPO-27(Zn), CPO-27(Co), CPO-27(Mg), CPO-27(Ni), Cr₃(BTC)₂, Cu(BDC-OH), CuBTC, CuTPTC, Cu₂(BPTC)(H₂O), CuTATB-30, CuTATB-60, DO-MOF, DTO-MOF, DUT-5, DUT-6, DUT-6(Zn), DUT-8(Ni), Dy(BTC)(DMF)₂, Dy(TATB)(H₂O), Er(BTC)(H₂O), Er(TATB)(H₂O), Eu(1,3,5-BTC), Eu(BTC)(H₂O), Eu(TATB)(H₂O), Eu(TPA)(H₂O), Eu(TPA)(FA), Eui-xTbx-MOFs, Fe-BTC, Ga-Im, Gd(BTC)(H₂O), Gd(TATB)(H₂O), Gd(TPA)(FA), HKUST-1, HZIF-1(Mo), HZIF-1(W), Ho(BTC)(H₂O), Ho(TATB)(H₂O), IM-22, IRMOF-1, IRMOF-2, IRMOF-3, IRMOF-4, IRMOF-5, IRMOF-6, IRMOF-7, IRMOF-8, IRMOF-9, IRMOF-10, IRMOF-11, IRMOF-12, IRMOF-13, IRMOF-14, IRMOF-15, IRMOF-16, In-BTC, In-NDC, MAF-4, MCF-27, MIL-45(Co), MIL-45(Fe), MIL-47, MIL-53(Al), MIL-53(Sc), MIL-53(Cr), MIL-53(Fe), MIL-53(Ga), MIL-53(Sc), MIL-69, MIL-78, MIL-78(Y,Eu), MIL-88(Sc) MIL-88B, MIL-88B(2OH), MIL-88C(Cr), MIL-88C(Fe), MIL-88D, MIL-96, MIL-100(AI), MIL-100(Sc), MIL-100(Fe), MIL-101(Al), MIL-101(Cr), MIL-101 NDC, MIL-103, MIL-110, MIL-118, MIL-120, MIL-121, MIL-122, Mg₃(NDC)₃, Mg/DOBC, Mg₃(BHTC)₂, Mg(BPT)₂(H₂O)₄, MgDOBC, Mn(BDC)(H₂O)₂, Mn₃(BHTC)₃, MnSo-MOF, MOF-1, MOF-5, MOF-14, MOF-38, MOF-39, MOF-69B, MOF-74(Zn), MOF-74(Fe), MOF-74(Co), MOF-74(Mg), MOF-74(Ni), MOF-143, MOF-177, MOF-200, MOF-205, MOF-235, MOF-399, MOF-501(Co), MOF-501, MOF-502, MOF-505, NENU-11, NENU-27, Ni₃(BTC)₂, NOTT-100, NOTT-101, NOTT-102, NOTT-103, NOTT-104, NOTT-105, NOTT-106, NOTT-107, NOTT-108, NOTT-109, NOTT-110, NOTT-111, NOTT-112, NOTT-113, NOTT-113, NOTT-115, NOTT-116, NOTT-119, NOTT-140, NOTT-200, NOTT-201, NOTT-204, NOTT-205, NOTT-206, NOTT-207, NOTT-208, NOTT-209, NOTT-210, NOTT-211, NOTT-212, NOTT-213, NOTT-300(Al), NOTT-300(Ga), NOTT-400, NOTT-401, Nd(BTC)(H₂O), Ni(BDC), Ni/DOBDC, Ni₃(BTC)₂, NiDOBDC, PCN-5, PCN-6, PCN-6′, PCN-9(Co), PCN-9(Fe), PCN-9(Mn), PCN-12, PCN-12′, PCN-13, PCN-17(Dy), PCN-17(Er), PCN-17(Y), PCN-17(Yb), PCN-19, PCN-131, PCN-131′, PCN-132, PCN-132′, RPF-12, RPF-13, RPF-14, Sc₂BDC₃, Sc₂(NH₂-BDC)₃, SCIF-1, SCIF-2, SNU-MI0, SNU-M11, SNU-M11(Ni), Sm(BTC)(H₂O), Sr, Eu-Im, TIF-2, TIF-A1, TIF-A2, TIF-A1(Zn), TIF-A2(Zn), TO-MOF, TUDMOF-1, TUDMOF-3, Tb(BTC)(DMF)₂, Tb(BTC)(H₂O), Tb(TATB)(H₂O), Tb(TPA)(FA), Tb-BTC, Tm(BTC)(DMF)₂, UIO-66(Zr), UL-MOF-1, UMCM-1, UMCM-2, UMCM-150, UTSA-25a, UTSA-35, UTSA-36, UTSA-38, UiO-66(Zr), UiO-66-X, Y(TATB)(H₂O), Y-BTC, YO-MOF, Yb(BTC)(H₂O), Yb(BTC)(DMF)₂, ZBIF-1, ZBIF-1(Zn), ZIF-1, ZIF-2, ZIF-3, ZIF-4, ZIF-8, ZIF-10, ZIF-60, ZIF-61, ZIF-62, ZIF-64, ZIF-65, ZIF-67, ZIF-70, ZIF-76, Zn(Im)(alm), Zn-IM, Zn/DOBTC, Zn₂(BTC), Zn₃(BTC)₂, Zn₃(NDC)₃, ZnPO-MOF, [Ag₄(HBTC)₂], [Cd₃(TATB)₂], [Mn₃(TATB)₂], nZIF-8, p-BDC-Co, porph@MOM-10, rho-ZMOF, and sod-ZMOF.

In one embodiment, the metal-organic framework is a chromium metal-organic framework, an iron metal-organic framework, or a zirconium metal-organic framework. In another embodiment, the metal-organic framework is MIL-101(Cr), PCN-333(Cr), PCN-333(Fe), Tb-mesoMOF, MOF-74-III, PCN-777, PCN-69, Zr-UiO-68, Zr-UIO-67-8F, UiO-68, MOF818, FDM-3, Tb-TATB, MIL-101-4F, or MIL-101-Br(Cr).

Given the bulky size of frustrated Lewis pairs (FLPs), metal-organic frameworks as used in the present disclosure should have different pore sizes, with large pores to accommodate frustrated Lewis pairs and small pores for reactants and products diffusion. In some embodiments, frustrated Lewis pairs can be incorporated into two classes of mesoporous metal-organic frameworks (mesoMOFs): mesoMOFs consisting of hierarchical nanospaces of different window sizes and mesoMOFs include interconnected channels of different apertures. In some embodiments, certain approaches can be employed to create a protective environment within the metal-organic framework to boost the water/moisture tolerance of the frustrated Lewis pair, a common issue for homogeneous frustrated Lewis pair systems.

### Step-Wise Anchoring of Frustrated Lewis Pairs (FLPs) in Metal-Organic Frameworks (MOFs) Through Coordination Interactions

In one aspect, a process is provided for the preparation of a composition as described herein comprising: providing a porous metal-organic framework (MOF) having at least one nanospace; contacting the metal-organic framework with a Lewis base such that the Lewis base is contained within the nanospace to form a Lewis base-MOF adduct; and contacting the Lewis base-MOF adduct with a Lewis acid, such that the Lewis base and the Lewis acid form a frustrated Lewis pair within the nanospace of the metal-organic framework. The metal-organic includes a plurality of metal sites with a plurality of nanospaces.

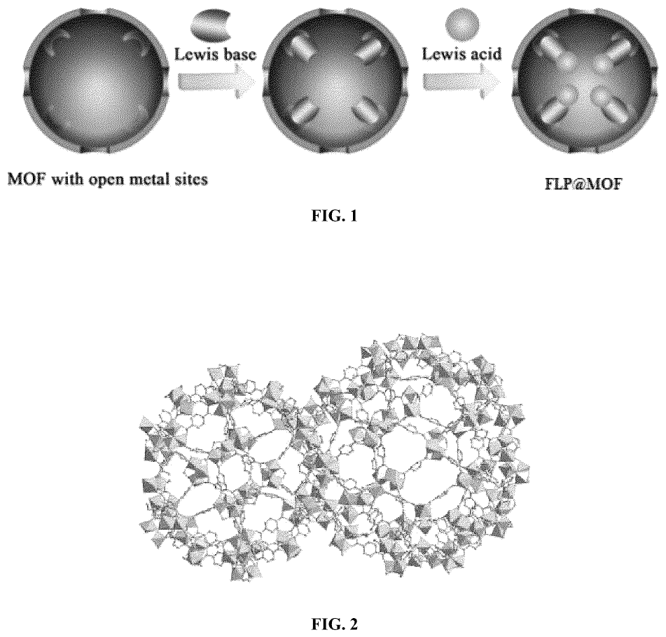

The solvent molecules on the secondary building units of a metal-organic framework can be liberated to expose metal sites allowing for ready coordination with functional ligands via a postsynthetic modification process. To employ step-wise anchoring of a frustrated Lewis pair into a metal-organic framework, the base moiety of frustrated Lewis pair is first anchored to the open metal sites within the metal-organic framework through coordination interaction, which is followed by the introduction of the corresponding acid moiety of the frustrated Lewis pair (see FIG. 1). Given the strong coordination interaction, it is anticipated that the frustrated Lewis pair would be stabilized within the metal-organic framework yet accessible to substrates.

In one embodiment, the Lewis base can bond with a plurality metal sites within a metal-organic framework. The mode of bonding between the metal site and Lewis base can depend upon the nature of the metal and Lewis base. Modes of bonding include, but are not limited to, covalent bonding, dative bonding, hydrogen bonding, or Van der Waals bonding.

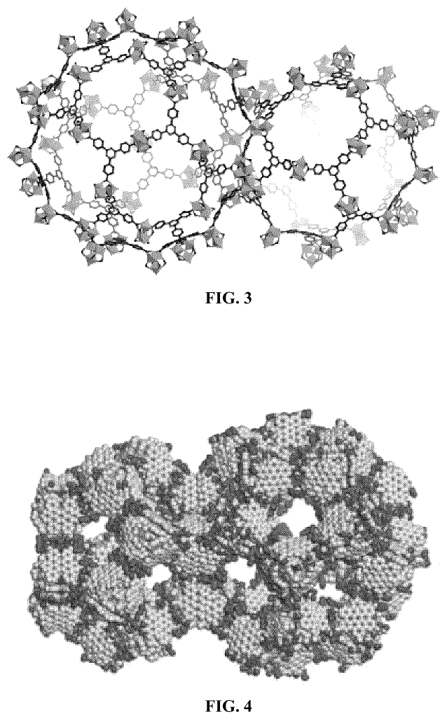

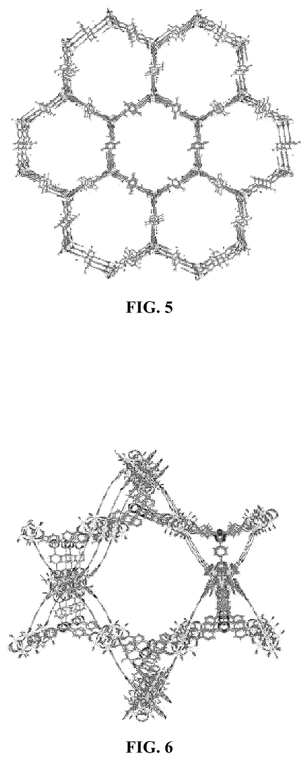

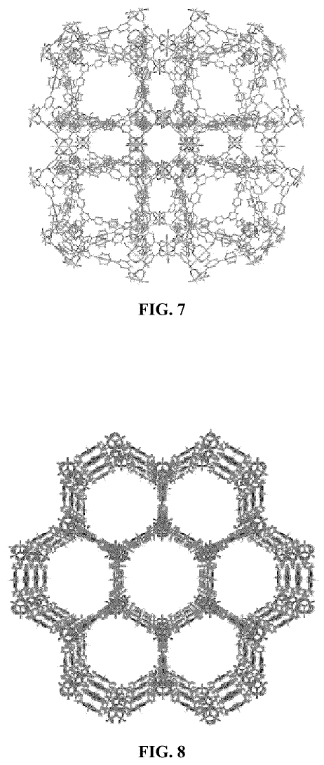

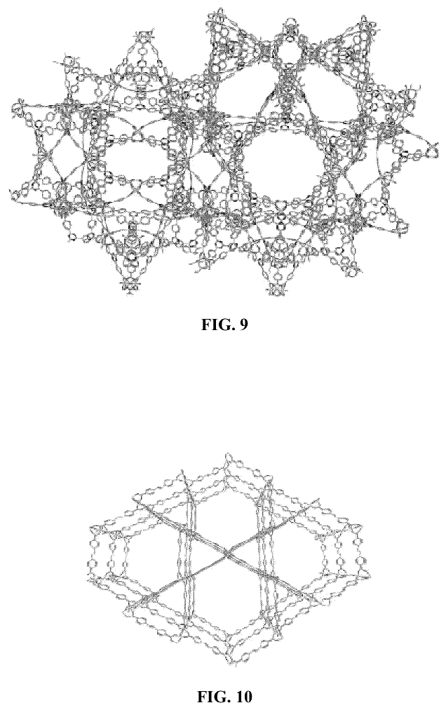

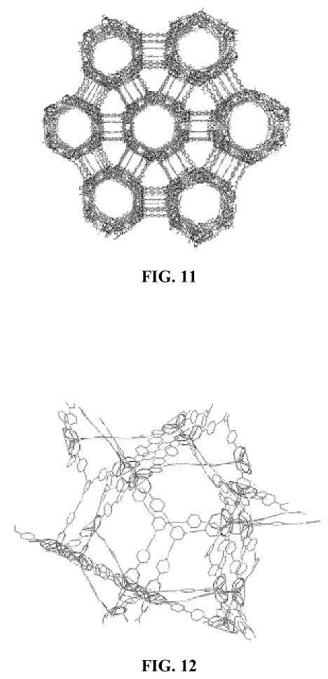

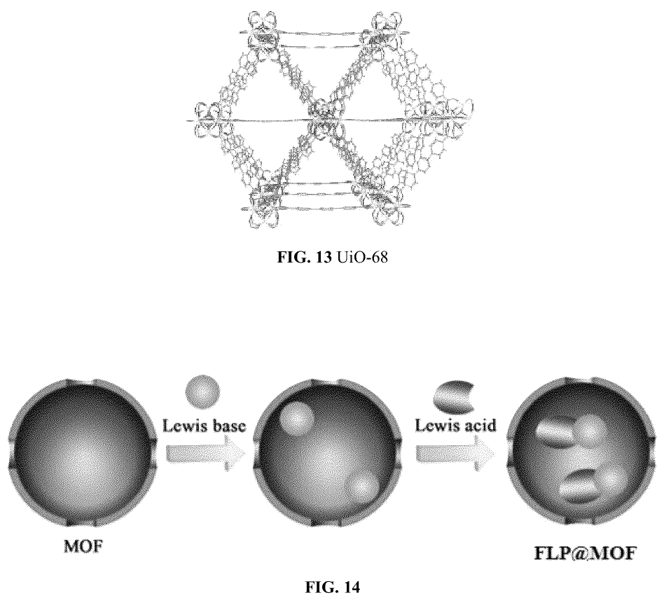

In one embodiment, the Lewis base is a N-ligand that has two or more potential nitrogen coordination centers: one can be used to coordinate with an open metal site serving as a linker meanwhile the other can react with the Lewis acid to form frustrated Lewis pair, playing the role of a Lewis base. For example, employing the step-wise anchoring strategy, the Lewis pair (e.g., DABCO and B(C₆F₅)₃) can be incorporated into the metal-organic framework (e.g., MIL-101(Cr). In another embodiment, PCN-333(Cr) (FIGS. 3 and 9) can be used to bind frustrated Lewis pairs. Particular N-ligands can be selected as Lewis base linkers for step-wise anchoring of frustrated Lewis pairs into MIL-101(Cr) and PCN-333(Cr). Additional examples of metal-organic frameworks that may be used for step-wise anchoring of the frustrated Lewis pair include MOF-74-III, PCN-69, PCN-600, and PCN-777.

Representative examples of Lewis base linkers for step-wise anchoring of frustrated Lewis pairs into metal-organic frameworks include, but are not limited to:

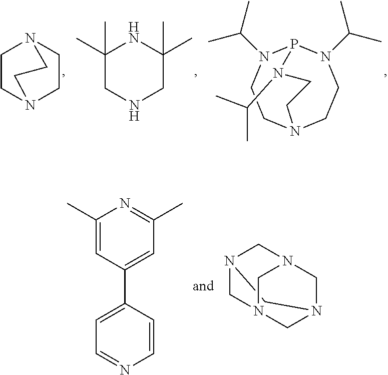

The amount of the frustrated Lewis base pair that can be incorporate into the porous material can vary depending upon the selection of the Lewis acid, Lewis base, porous material, and end-use of the composition. In one embodiment, the frustrated Lewis pair is in the amount of from about 0.1 mmol to about 5 mmol per 1 g of porous material, or about 0.1 mmol, about 0.5 mmol, about 1.5 mmol, about 2.0 mmol, about 2.5 mmol, about 3.0 mmol, about 3.5 mmol, about 4.0 mmol, about 4.5 mmol, or about 5.0 mmol per 1 g of porous material, where any value can be a lower and upper end-point of a range (e.g., about 0.5 mmol to about 2.0 mmol per 1 g of porous material).

### Assembly of Frustrated Lewis Pairs Within the Nanospace of Metal-Organic Frameworks

In another aspect, a process is provided for the preparation of a composition as described herein comprising: providing a porous metal-organic framework (MOF) having at least one nanospace; contacting the metal-organic framework with a Lewis base precursor such that the Lewis base precursor is contained within the nanospace to form a Lewis base precursor-metal-organic framework adduct; and contacting the Lewis base precursor-MOF adduct with a Lewis acid precursor such that the Lewis base precursor and the Lewis acid precursor react to form the frustrated Lewis pair within the nanospace of the metal-organic framework.

In one embodiment, a method to incorporate a frustrated Lewis pair in a metal-organic framework involves encapsulation of a frustrated Lewis pair as a guest molecule into the nanospace of the metal-organic framework. In one embodiment, the Lewis base component and Lewis acid entity of the frustrated Lewis pair are sequentially delivered into a cage-containing mesoMOF with window sizes smaller than the molecular dimension of frustrated Lewis pair yet large enough to permit the entrance of individual Lewis base and Lewis acid components and the cage room spacious enough allowing for the formation and accommodation of the bulky frustrated Lewis pair molecule (FIG. 14). In addition to heterogenizing the frustrated Lewis pair, with the frustrated Lewis pair residing as guest within the nanospace of the metal-organic framework, it is possible to create cooperativity between the frustrated Lewis pair and metal-organic framework framework thus to further facilitate the activation of dihydrogen. In addition, such a process would permit a high diffusion rate of the individual Lewis base and Lewis acid components in the nanospace of the metal-organic framework to accommodate bulky frustrated-Lewis pair molecules. Representative examples of metal-organic frameworks that can be employed with this approach include MIL-101(Cr) having window sizes of ˜1.2 nm/˜1.4 nm and cage diameters of 2.9 nm/3.3 nm (FIG. 2), PCN-333 possessing ˜2.6 nm/3.0 nm sized windows and 4.2 nm/5.5 nm diameter cages (FIGS. 3 and 9), and Tb-mesoMOF featuring ˜1.3 nm/1.7 nm window apertures and ˜3.9 nm/4.7 nm diameter cavities (FIG. 4) as platforms, to de novo assemble a series of selected intramolecular frustrated Lewis pairs that can be formed by their Lewis base and Lewis acid fragments under mild reaction conditions within their nanospace for dihydrogen activation. Additional metal-organic frameworks that may be used by this approach include CYCU-3, Bio-MOF-100, MOF-210, and UiO-68. Representative examples of frustrated Lewis pairs that may be formed within the nanospace of the metal-organic framework using this approach include:

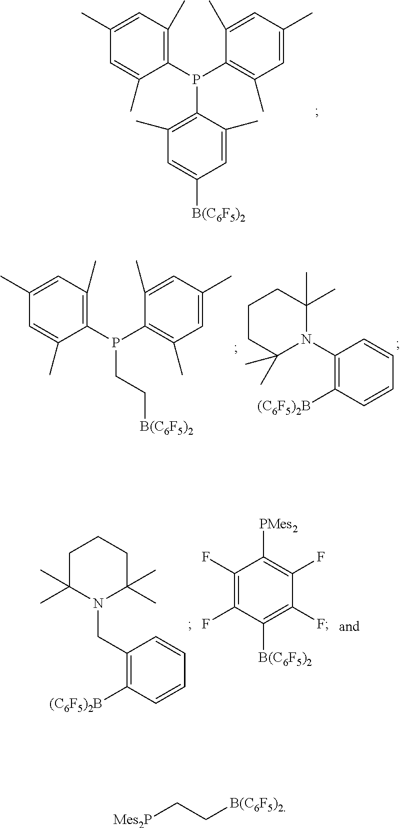

### Assembly of Frustrated Lewis Pair Impregnated Metal-Organic Frameworks Using Lewis-Base Containing Ligands

In another aspect, a composition is provided comprising: a porous metal-organic framework having at least one nanospace and comprising one or more metal ions and one or more organic linker ligands; and a frustrated Lewis pair comprising a Lewis base and a Lewis acid; wherein the Lewis base is covalently bound to at least one of the one or more organic linker ligands; and wherein the frustrated Lewis pair is contained within at least one nanospace of the metal-organic framework.

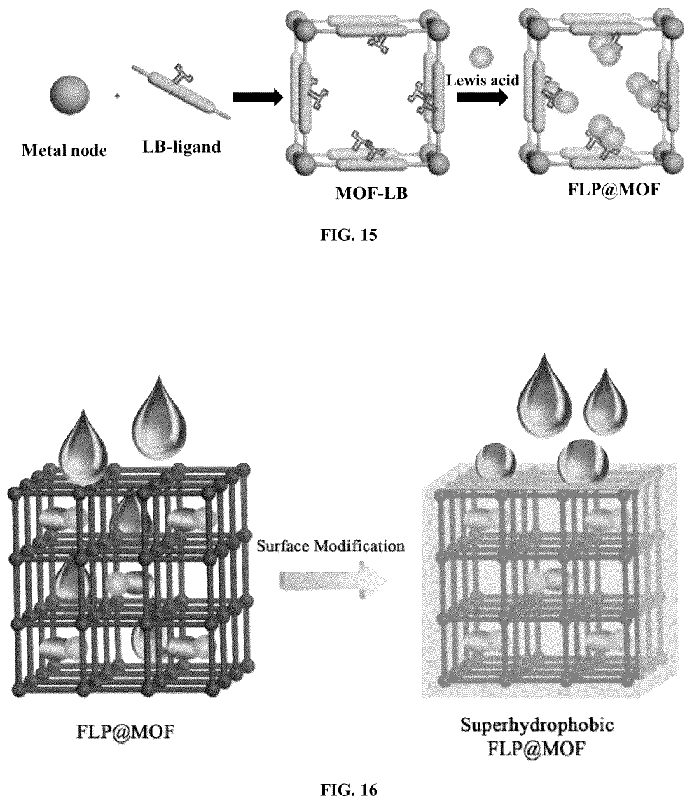

Another method provided herein for the incorporation of frustrated Lewis pairs into metal-organic frameworks is to use a Lewis base containing organic linker ligand for the construction of the metal-organic framework structure. Given the instability of most Lewis acid components of frustrated Lewis pairs, a typical embodiment includes having the ligand functionalized with a Lewis base moiety to construct the metal-organic framework and followed by subsequent addition of the Lewis acid moiety to form the desired composition (FIG. 15). Considering the high stability and framework robustness of zirconium-based metal-organic frameworks (Zr-MOFs), one embodiment includes utilizing a series of organic linker ligands featuring Lewis base moieties as described herein to construct Zr-MOFs followed by the addition of corresponding Lewis acid moieties to form FLPs@Zr-MOFs for use in dihydrogen activation. Representative examples of organic linker ligands for metal-organic framework synthesis that contain covalently bound lewis base moieties include, but are not limited to:

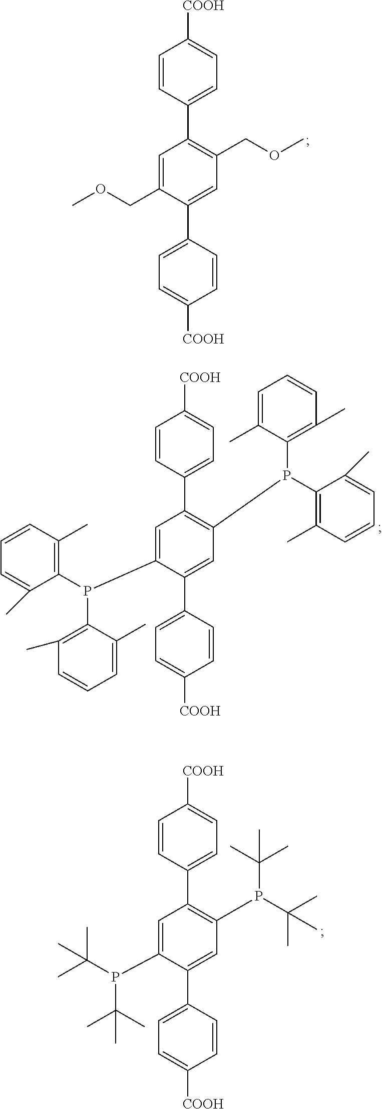

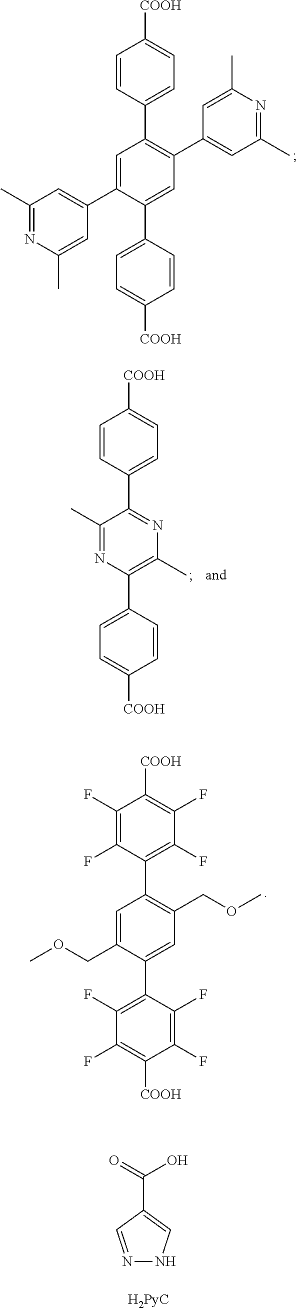

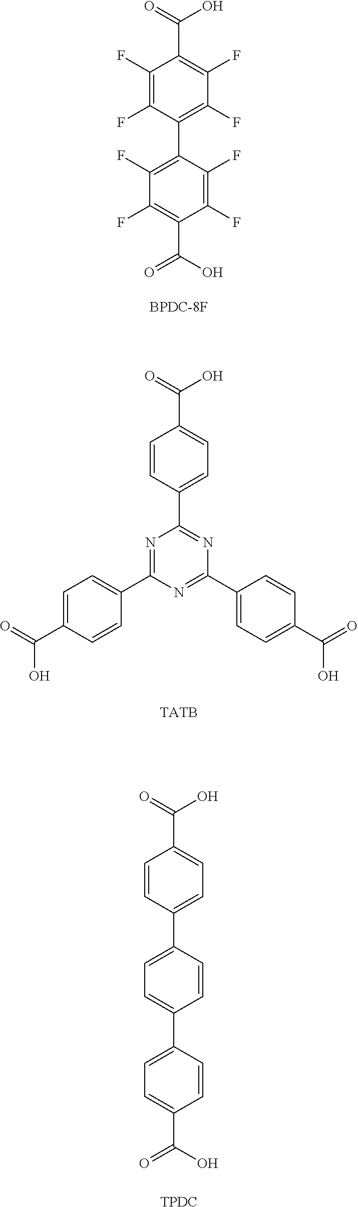

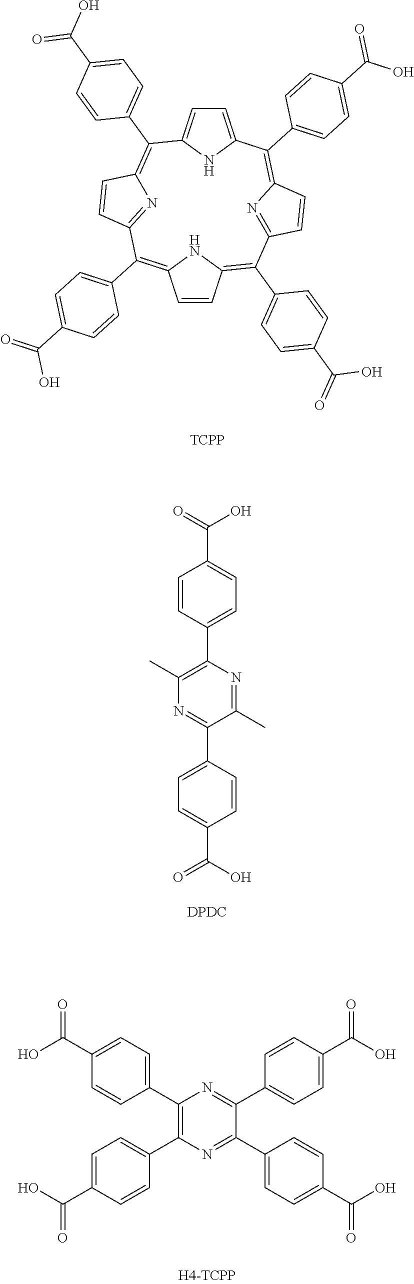

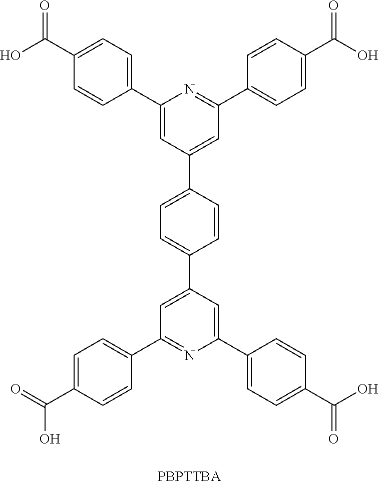 [SMILES: O=C(O)c1ccc(-c2cc(-c3ccc(-c4cc(-c5ccc(C(=O)O)cc5)nc(-c5ccc(C(=O)O)cc5)c4)cc3)cc(-c3ccc(C(=O)O)cc3)n2)cc1]

### Superhydrophobic Metal-Organic Frameworks for Increased Moisture Tolerance of Impregnated Frustrated Lewis Pairs

As frustrated Lewis pairs are known for instability toward water/moisture, one embodiment of the present disclosure aims to create a protective environment against water molecules within the metal-organic framework. Two approaches can be employed to create a superhydrophobic environment for frustrated Lewis pair-impregnated metal-organic frameworks. One aspect includes the use of a superhydrophobic ligand to construct the metal-organic framework with a superhydrophobic nanospace that is capable of both precluding the entrance of the water molecules and expelling the water generated during the reaction, thereby preventing hydrolytic degradation of the frustrated Lewis pair. Another aspect includes decorating the exterior surface of the metal-organic framework with long chain perfluoroalkyl groups to afford a super-hydrophobic exterior surface without sacrificing the surface area and pore size/volume yet serving as a shield to resist water and thus to protect the frustrated Lewis pair incorporated within the metal-organic framework (FIG. 16). Such super-hydrophobic compositions can be used for hydrogenation catalysis reactions when water is generated in situ or the solvents contain trace amount of water. A representative example of an organic linker ligand that may be used in these embodiments directed to superhydrophobic metal-organic frameworks includes, but is not limited to:

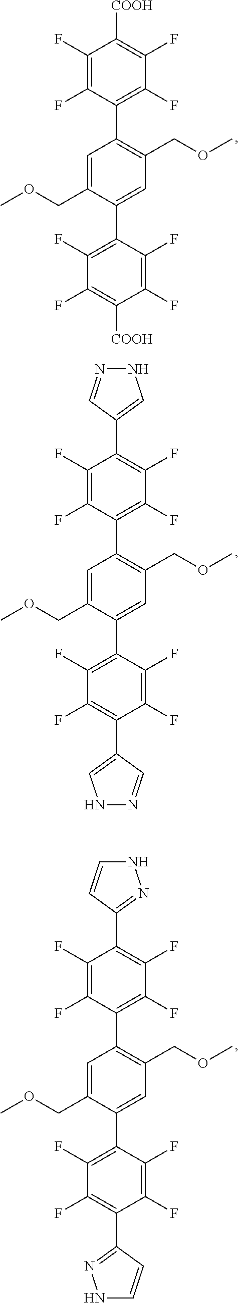

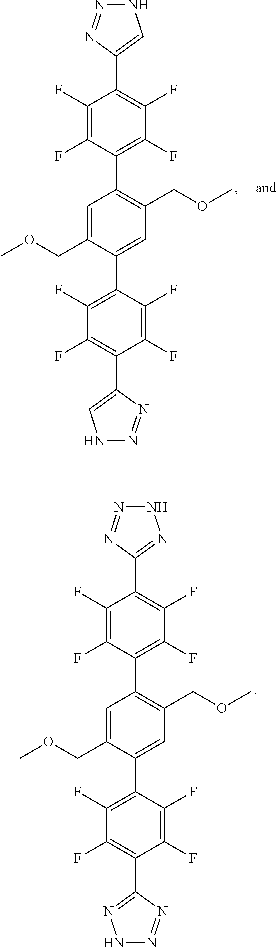

The porous material can have an outer surface functionalized with perfluoroalkyl moieties. By selecting perfluoroalkyl moieties that will not permeate into the pores of the porous material, for example a metal-organic framework, essentially just the outer surface of the porous material is functionalized. A variety of perfluoroalkyl moieties can be used in this regard. The perfluoroalkyl moiety can be a linear or branched chain fluorinated alkyl group having from 7 to 20, 8 to 20, 9 to 20, or 10 to 20 carbon atoms. In one aspect, the organic linker ligands used in the synthesis of a metal-organic framework as used in the present disclosure may have one or more reactive coupling groups that may react with a second functional group on the perfluoroalkyl moieties to form a covalent bond. In some embodiments, the reactive coupling group can be a primary amine and the second functional group may be selected from an isocyanate, isothiocyanate, acyl azide, NHS ester, sulfonyl chloride, aldehyde, glyoxal, epoxide, oxirane, carbonate, aryl halide, imidoester, carbodiimide, anhydride, or fluorophenyl ester. In other embodiments, the reactive coupling group can be a thio group and the second functional group may be selected from a maleimide, haloacetyl, or pyridyl disulfide group. In some embodiments, the reactive coupling group can be a photoreactive coupling group such as an aryl azide or a diazirine. In some embodiments, the reactive coupling group can be an alkene that subsequently reacts with a thiol group on the perfluoroalkyl moieties to form a covalent bond.

In one embodiment, the superhydrophobic porous material as can be used in the present compositions is selected from one described in WO 2018/129406, incorporated herein by reference in its entirety.

### Exemplary Compositions

In one aspect, the metal organic framework is MOF818, FDM-3, Tb-TATB, Zr-UIO-68, Zr-UIO-67-8F, PCN-333(Fe), PCN-333(Cr), MIL-101(Cr), MIL-101-4F, or MIL-101-Br(Cr), the Lewis acid is B(C₆F₅)₃, and the Lewis base is 1,4-diazabicyclo[2.2.2]octane (DABCO).

In one aspect, the metal organic framework is MOF818, FDM-3, Tb-TATB, Zr-UIO-68, Zr-UIO-67-8F, PCN-333(Fe), PCN-333(Cr), MIL-101(Cr), MIL-101-4F, or MIL-101-Br(Cr), the Lewis acid is B(C₆F₅)₃, and the Lewis base is hexamethylenetetramine (HMTA).

In one aspect, the metal organic framework is MOF818, FDM-3, Tb-TATB, Zr-UIO-68, Zr-UIO-67-8F, PCN-333(Fe), PCN-333(Cr), MIL-101(Cr), MIL-101-4F, or MIL-101-Br(Cr), the Lewis acid is MesB(C₆F₅)₃, and the Lewis base is 1,4-diazabicyclo[2.2.2]octane (DABCO).

In one aspect, the metal organic framework is MOF818, FDM-3, Tb-TATB, Zr-UIO-68, Zr-UIO-67-8F, PCN-333(Fe), PCN-333(Cr), MIL-101(Cr), MIL-101-4F, or MIL-101-Br(Cr), the Lewis acid is MesB(C₆F₅)₃, and the Lewis base is hexamethylenetetramine (HMTA).

In one aspect, the metal organic framework is not MIL-101(Cr). In another aspect, the composition is not MIL-101(Cr), where the Lewis acid is B(C₆F₅)₃, and the Lewis base is 1,4-diazabicyclo[2.2.2]octane (DABCO).

### Hydrogenation Methods

In another aspect, methods are provided for the hydrogenation of organic compounds using the compositions described herein. Thus, in one aspect, a process is provided for the hydrogenation of an organic compound with at least one point of unsaturation comprising: providing the organic compound; providing the composition as described herein; and contacting the organic compound with the composition in the presence of hydrogen gas. The organic compound that may be hydrogenated by the methods described herein typically will have at least one point of unsaturated, typically a functional group that contains such a point of unsaturation. Representative examples of functional groups that may be hydrogenated using the methods described herein include, but are not limited to, imines, ketimines, aldimines, quinolines, phenanthrolines, alkynes, alkenes, enamines, silyl enol ethers, enones (either C═C or C—O reduction), ketones, aldehydes, indoles, thienes, and polyaromatic hydrocarbons. The term “hydrogenation” means to treat a compound with hydrogen typically to cause an addition of hydrogen across a point of unsaturation in the compound without cleaving bonds. In another aspect, methods are also provided for the hydrogenolysis of organic compounds using the compositions described herein. The term “hydrogenolysis” refers to a chemical reaction whereby a carbon-carbon or carbon-heteroatom bond (typically a C—O, C—N, C—S, or C-halo bond) is cleaved and undergoes breakdown by hydrogen.

Non-limiting representative embodiments of the use of the compositions provided herein in hydrogenation and hydrogenolysis reactions are further provided below.

### C═N Bond Reduction Using Frustrated Lewis Pair Impregnated Metal-Organic Frameworks

In one aspect, a process is provided for hydrogenation of an organic compound containing at least one imine functional group, wherein the imine functional group is converted into an amino functional group, comprising contacting the organic compound with a composition as described herein (FLP@MOF) in the presence of hydrogen gas. In some embodiments, the above process is as provided in the below generic scheme:

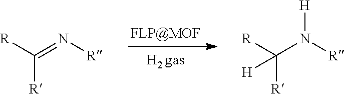

In one embodiment, the frustrated Lewis pair as used in above imine hydrogenation process includes a Lewis acid selected from

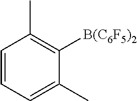

and a Lewis base selected from

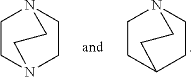 [SMILES: C1CN2CCN1CC2 ; C1CN2CCC1CC2]

In another embodiment, the frustrated Lewis pair as used in the above imine hydrogenation process includes a Lewis acid selected from

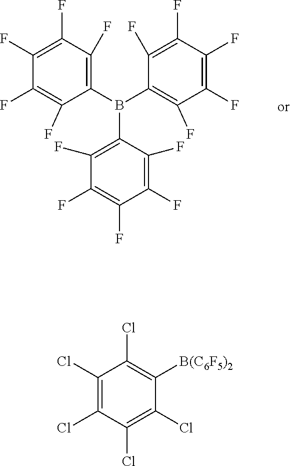

and the Lewis base selected from tetrahydrofuran.

In another embodiment, the frustrated Lewis pair as used in the above imine hydrogenation process is selected from

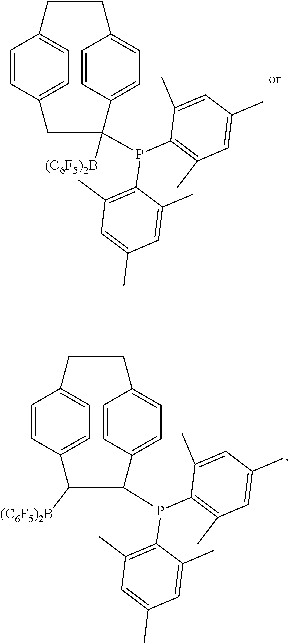

### C—C Double Bond Reduction Using Frustrated Lewis Pair Impregnated Metal-Organic Frameworks

In another aspect, a process is provided for hydrogenation of an organic compound containing at least one alkenyl functional group, wherein the alkenyl functional group is converted into an alkyl functional group, comprising contacting the organic compound with a composition as described herein (FLP@MOF) in the presence of hydrogen gas. In some embodiments, the above process is as provided in the below generic scheme:

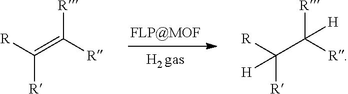

In one embodiment, the frustrated Lewis pair as used in the above alkene hydrogenation process includes a Lewis acid selected from

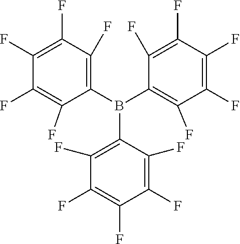 [SMILES: Fc1c(F)c(F)c(B(c2c(F)c(F)c(F)c(F)c2F)c2c(F)c(F)c(F)c(F)c2F)c(F)c1F]

and a Lewis base selected from diethyl ether,

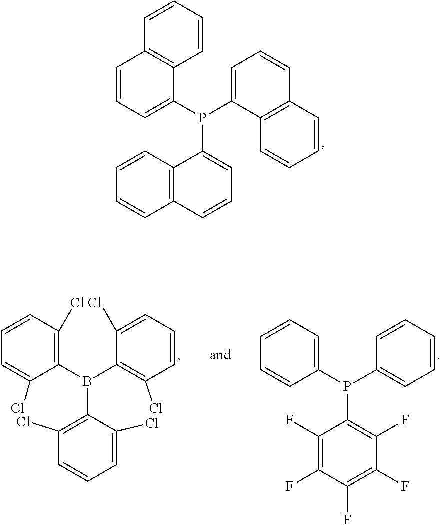 [SMILES: c1ccc2c(P(c3cccc4ccccc34)c3cccc4ccccc34)cccc2c1 ; Clc1cccc(Cl)c1B(c1c(Cl)cccc1Cl)c1c(Cl)cccc1Cl ; Fc1c(F)c(F)c(P(c2ccccc2)c2ccccc2)c(F)c1F]

In one embodiment, the frustrated Lewis pair as used in the above alkene hydrogenation process includes a Lewis acid selected from

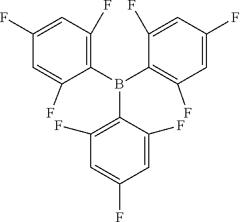 [SMILES: Fc1cc(F)c(B(c2c(F)cc(F)cc2F)c2c(F)cc(F)cc2F)c(F)c1]

and a Lewis base selected from

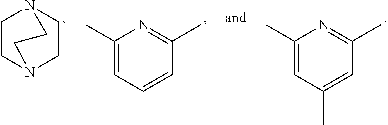

In one embodiment, the frustrated Lewis pair as used in the above alkene hydrogenation process includes a Lewis acid selected from

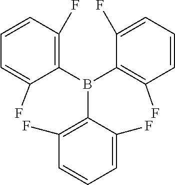 [SMILES: Fc1cccc(F)c1B(c1c(F)cccc1F)c1c(F)cccc1F]

and a Lewis base selected from

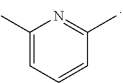

In one embodiment, the frustrated Lewis pair as used in the above alkene hydrogenation process includes a Lewis acid selected from

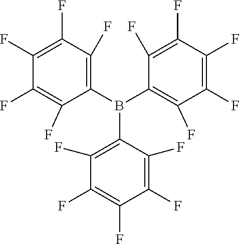 [SMILES: Fc1c(F)c(F)c(B(c2c(F)c(F)c(F)c(F)c2F)c2c(F)c(F)c(F)c(F)c2F)c(F)c1F]

and a Lewis base selected from

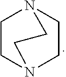

### C═C Triple Bond Reduction Using Frustrated Lewis Pair Impregnated Metal-Organic Frameworks

In another aspect, a process is provided for hydrogenation of an organic compound containing at least one alkenyl functional group, wherein the alkynyl functional group is converted into an alkenyl functional group or an alkyl functional group, comprising contacting the organic compound with a composition as described herein (FLP@MOF) in the presence of hydrogen gas. In some embodiments, the above process is as provided in the below generic scheme:

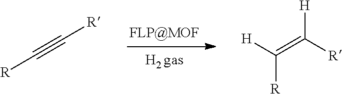

In one embodiment, the frustrated Lewis pair as used in the above alkyne reduction process includes:
a Lewis acid selected from

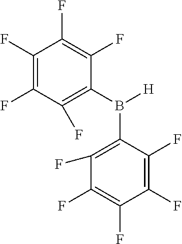 [SMILES: Fc1c(F)c(F)c(Bc2c(F)c(F)c(F)c(F)c2F)c(F)c1F]

and a Lewis base selected from

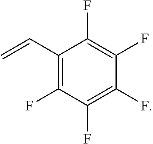

In one embodiment, the frustrated Lewis pair as used in the above alkyne reduction process is selected from

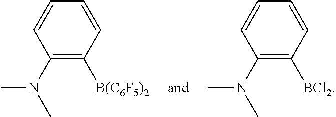

C═O Double Bond Reduction and Reductive Deoxygenation using Frustrated Lewis Pair Impregnated Metal-Organic Frameworks

In another aspect, a process is provided for hydrogenation of an organic compound containing at least one carbonyl functional group selected from an aldehyde or ketone, wherein the carbonyl functional group is converted into an alcohol functional group, comprising contacting the organic compound with a composition as described herein (FLP@MOF) in the presence of hydrogen gas. In some embodiments, the above process is as provided in the below generic scheme:

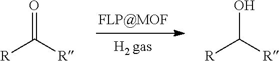

In another aspect, a process is provided for hydrogenation of an organic compound containing at least one carbonyl functional group selected from an aldehyde or ketone, wherein the carbonyl functional group is converted into a methylene group, comprising contacting the organic compound with a composition as described herein in the presence of hydrogen gas. In some embodiments, the above process is as provided in the below generic scheme:

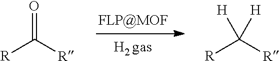

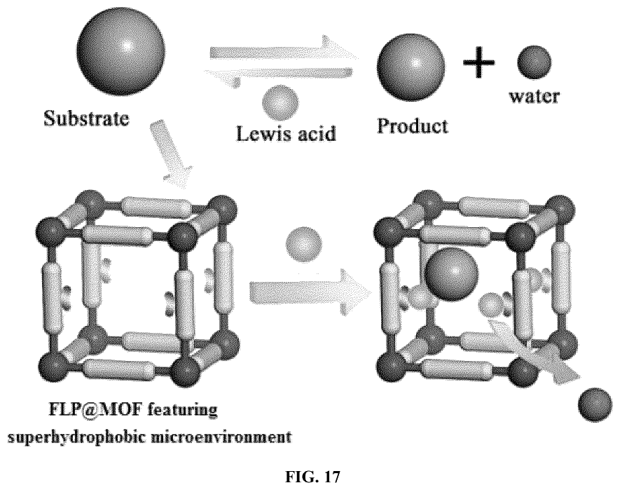

In one embodiment, by creating a superhydrophobic microenvironment within the metal-organic framework pore space as described herein (FIG. 17), the water generated in situ can be easily expelled, thus facilitating the reductive deoxygenation of aldehydes and ketones by the same frustrated Lewis pair-impregnated metal-organic framework. To prevent quenching of the open metal site on the metal-organic framework, a non-coordination solvent, such as toluene, is typically used for these reactions in some embodiments.

In some embodiments, the frustrated Lewis pair as used in the above carbonyl hydrogenation or hydrogenolysis process includes a Lewis acid selected from

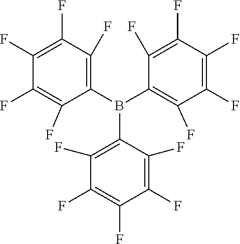 [SMILES: Fc1c(F)c(F)c(B(c2c(F)c(F)c(F)c(F)c2F)c2c(F)c(F)c(F)c(F)c2F)c(F)c1F]

and a Lewis base selected from 1,4-dioxane or diethyl ether.

### Oxime Ether Reduction Using Frustrated Lewis Pair Impregnated Metal-Organic Frameworks

In another aspect, a process is provided for hydrogenation of an organic compound containing at least one oxime functional group, wherein the oxime functional group is converted into an alkoxy amine functional group or a hydroxyl amine functional group, comprising contacting the organic compound with a composition as described herein (FLP@MOF) in the presence of hydrogen gas. In some embodiments, the above process is as provided in the below generic scheme:

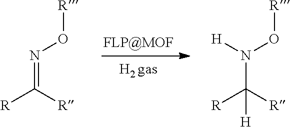

In one embodiment, the frustrated Lewis pair as used in the above oxime hydrogenation

process includes a Lewis acid selected from

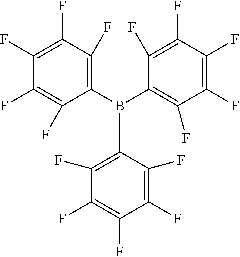 [SMILES: Fc1c(F)c(F)c(B(c2c(F)c(F)c(F)c(F)c2F)c2c(F)c(F)c(F)c(F)c2F)c(F)c1F]

and a Lewis base selected from 1,4-dioxane. In other embodiments, the oxime substrate may substitute for the Lewis base in the frustrated Lewis pair.

### Chemoselective Hydrogenation Using Frustrated Lewis Pair Impregnated Metal-Organic Frameworks

In another aspect, the hydrogenation methods using the compositions described herein can be used to selectively hydrogenate a first functional group in a compound that has two or more functional groups that might be subject to hydrogenation by other methods. In some embodiments, the first functional group is a carbonyl group (for example a ketone, aldehyde, ester, or amide group) or an imine group and a second functional group is an alkene. In one embodiment, the carbonyl or imine group and the alkene are conjugate, i.e. the compound contains an α,β-unsaturated carbonyl group or an α,β-unsaturated imine group, where the carbonyl group or imine group are selectively hydrogenated.

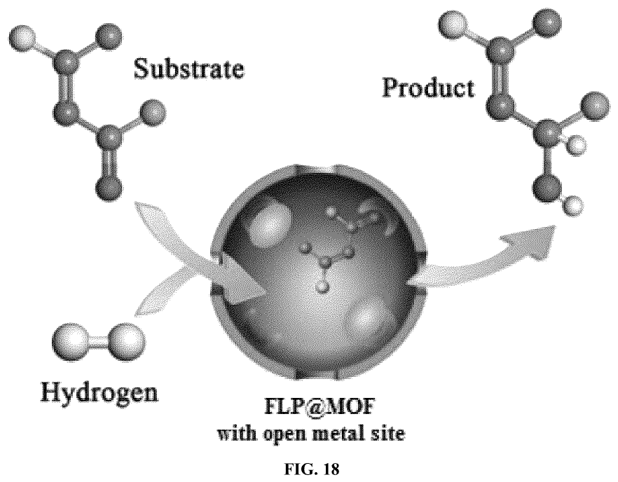

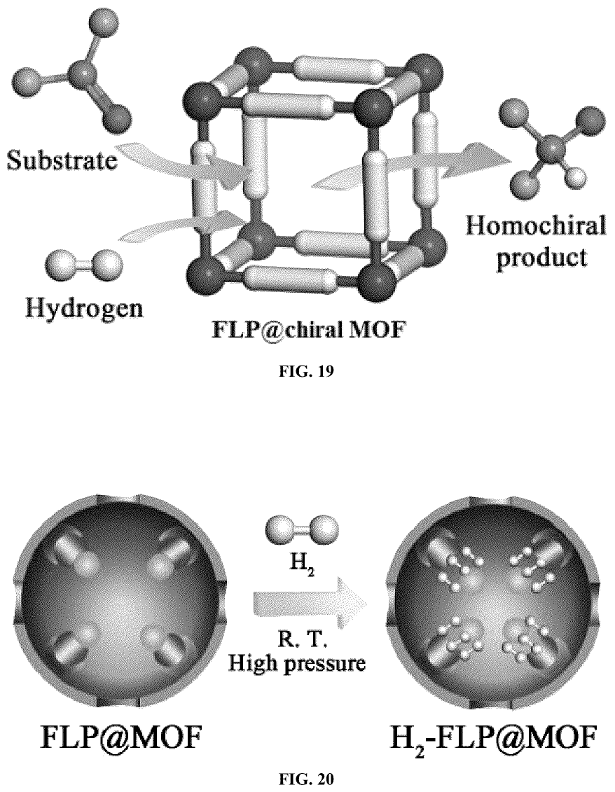

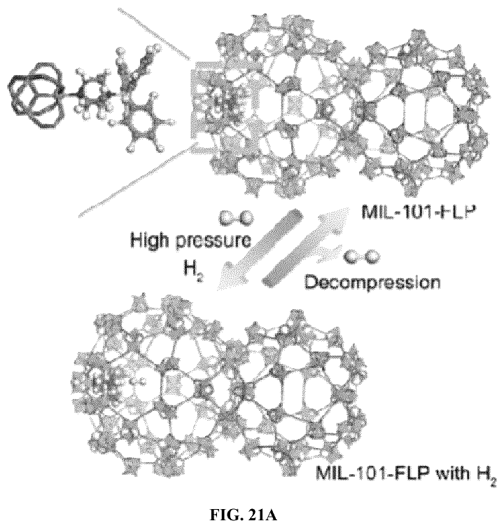

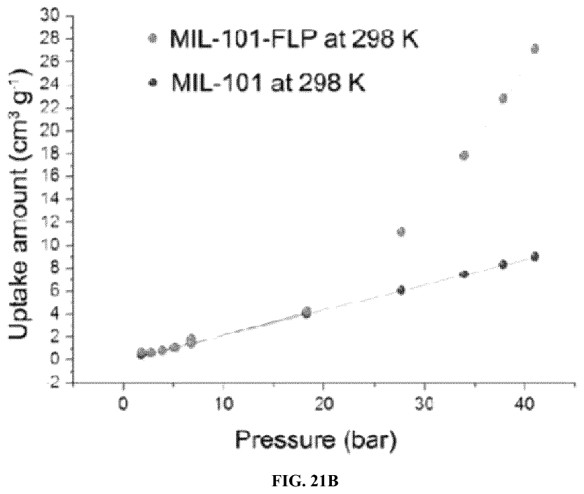

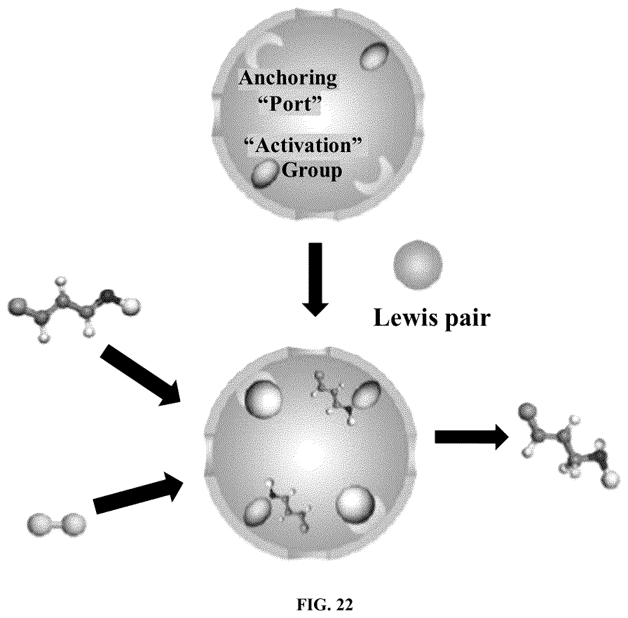

Chemo-selective hydrogenation, e.g. selective reduction of α, β-unsaturated carbonyl compounds has been a challenging topic since the early stage of organic chemistry. Despite the fact that 1,2 addition can be achieved by strong bases, such as hydride, catalytic reduction of the carbonyl is less reported especially in main group chemistry. Frustrated Lewis pair chemistry has been proven to be a promising way of replacing costly noble metal catalysts in terms of catalytic hydrogenations but has not been reported for selective reduction of carbonyl in α,β-unsaturated carbonyl compounds. Instead, alkene reduction of enone substrates is favored in almost all the cases reported thus far. With the compositions described herein, coordination facilitated chemo-selective reduction can be achieved by selective carbonyl binding to an open metal site of the metal-organic framework (FIG. 18).

### Chiral Hydrogenation Using Frustrated Lewis Pair Impregnated Metal-Organic Frameworks

In another aspect, compositions are provided comprising metal-organic frameworks formed from organic linker ligands that are chiral. These compositions, due to their inherent chirality, may be able to be used in effecting chiral hydrogenations of compounds that contain points of saturation that would be considered prochiral. Thus in another aspect, a composition is provided comprising: a porous metal-organic framework having at least nanospace and comprising one or more metal ions and one or more chiral organic linker ligands; and a frustrated Lewis pair comprising a Lewis base and a Lewis acid; wherein the Lewis base and/or Lewis acid are covalently bound to at least one of the one or more organic linker ligands; and wherein the frustrated Lewis pair is contained within at least one nanospace of the metal-organic framework. In an additional aspect, a process is provided for the asymmetric hydrogenation of an organic compound that contains a prochiral unsaturated functional group comprising contacting the organic compound with the chiral composition as described herein in the presence of hydrogen gas.

Viewed as one of the most challenging aspects for frustrated Lewis pair catalyst design, asymmetric hydrogenation catalyzed by frustrated Lewis pair still remains an unsolved problem despite several asymmetric frustrated Lewis pair systems having been developed for silyl enol ether, enamine, and ketimine reduction. The major difficulty lies in the accessibility of chiral catalysts, considering the huge amount of efforts required for chiral moiety design and structural variation in catalyst screening. The tremendous progress in chiral metal-organic frameworks over the past decade provides the ability to develop FLP@chiral MOFs for asymmetric hydrogenation. Two approaches can be employed for the development of FLP@chiral MOFs: one is to incorporate achiral frustrated Lewis pair into a chiral metal-organic framework that serves as a chiral scaffold to provide a strong chiral environment using the aforementioned anchoring and de novo assembly strategies, and this can circumvent the onerous work as required for chiral auxiliary synthesis in existing asymmetric frustrated Lewis pair systems; the other one is to use a chiral frustrated Lewis pair ligand to construct the chiral metal-organic framework structure.

Representative examples of chiral organic linker ligands that can be used to form a chiral composition as described herein include, but are not limited to:

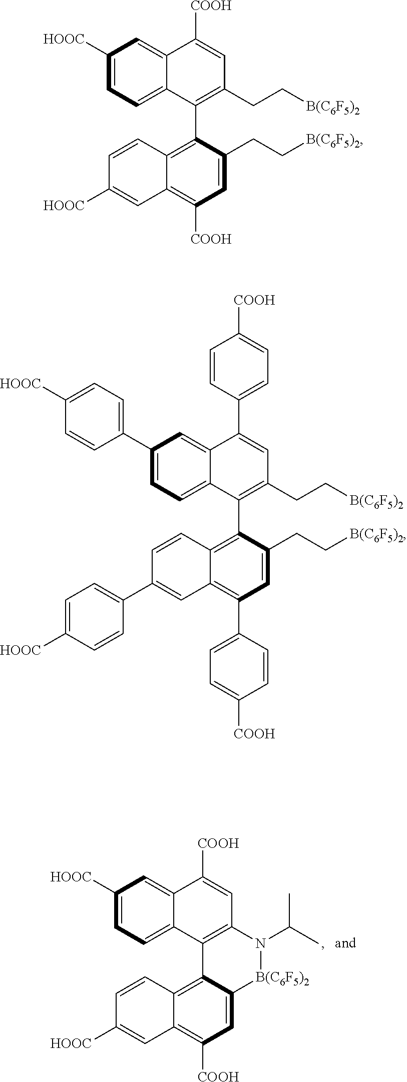

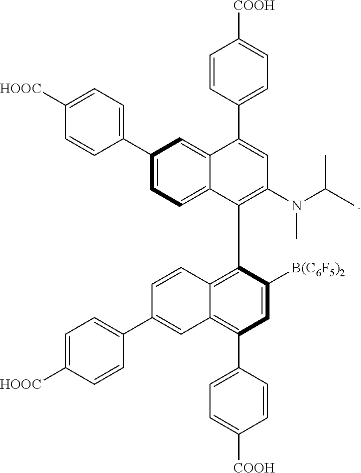

### Hydrogen Storage

In one aspect, the compositions described herein can be used to store hydrogen. Hydrogen storage technology has numerous application such as, for example, next generation transportation technology such as proton exchange membrane fuel cells. In one aspect, the compositions have a stable and regenerable gravimetric capacity greater than or equal 2.0 wt % and volumetric capacity greater than or equal 0.015 kg H₂/L under hydrogen pressure of less than or equal 200 bar at ambient temperature. In another aspect, the compositions have a stable and regenerable gravimetric capacity of about 2.0 wt % to about 10.0 wt % and volumetric capacity of about 0.015 kg H₂/L to about 0.100 kg H₂/L under hydrogen pressure of less than or equal 200 bar at ambient temperature. In another aspect, the compositions have a stable and regenerable gravimetric capacity of about 2.0 wt %, about 2.5 wt %, about 3.0 wt %, about 3.5 wt %, about 4.0 wt %, about 4.5 wt %, about 5.0 wt %, about 5.5 wt %, about 6.0 wt %, about 6.5 wt %, about 7.0 wt %, about 7.5 wt %, about 8.0 wt %, about 8.5 wt %, about 9.0 wt %, about 9.5 wt %, or about 10.0 wt % and volumetric capacity of about 0.015 kg H₂/L, about 0.020 kg H₂/L, about 0.025 kg H₂/L, about 0.030 kg H₂/L, about 0.035 kg H₂/L, about 0.040 kg H₂/L, about 0.045 kg H₂/L, about 0.050 kg H₂/L, about 0.055 kg H₂/L, about 0.060 kg H₂/L, about 0.065 kg H₂/L, about 0.070 kg H₂/L, about 0.075 kg H₂/L, about 0.080 kg H₂/L, about 0.085 kg H₂/L, about 0.090 kg H₂/L, about 0.095 kg H₂/L, or about 0.100 kg H₂/L under hydrogen pressure of less than or equal 200 bar at ambient temperature, where any value can be an endpoint of a range (e.g., about 4.0 wt % to about 5.0 wt %, about 0.020 kg H₂/L to about 0.040 kg H₂/L, etc.). In another aspect, the compositions described herein have hydrogen binding energies from about 15 kJ/mol to about 25 KJ/mol at a temperature between 20° C. to 30° C. (e.g., 25° C.).

### Aspects

The present disclosure can be described in accordance with the following numbered Aspects, which should not be confused with the claims.

Aspect 1: A composition comprising:
a porous material having at least one nanospace comprising a porous metal-organic framework (MOF) or a porous organic polymer (POP); and
a frustrated Lewis pair comprising a Lewis base and a Lewis acid;
wherein the frustrated Lewis pair is contained within at least one nanospace of the porous material.

Aspect 2: The composition of aspect 1, wherein the porous material is a porous metal-organic framework.

Aspect 3: The composition of aspect 1, wherein the porous material is a porous organic polymer.

Aspect 4: The composition of v 3, wherein the porous organic polymer is a porous aromatic framework.

Aspect 5: The composition of aspect 3, wherein the porous organic polymer is a porous polymer network.

Aspect 6: The composition of aspect 3, wherein the porous organic polymer is a covalent organic framework.

Aspect 7: The composition of any one of aspects 1 or 2, wherein the metal-organic framework comprises one or more metal ions and one or more organic linker ligands.

Aspect 8: The composition of aspect 7, wherein the one or more metal ions are an ion of a metal selected from Mg, Ca, Sr, Ba, Sc, Y, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Hg, Al, Ga, In, TI, Si, Ge, Sn, Pb, As, Sb, and Bi, or combinations thereof.

Aspect 9: The composition of any one of aspects 7 or 8, wherein the one or more organic linker ligands are selected from a polycarboxylate ligand, a polypyridyl ligand, a polycyano ligand, a polyphosphate ligand, a polyhydroxyl ligand, a polysulfonate ligand, a polyimidazolate ligand, a polytriazolate ligand, a polytetrazolate ligand, and a polypyrazolate ligand, or combinations thereof.

Aspect 10: The composition of any one of aspects 7-9, wherein the one or more organic linker ligands are selected from: 1,2,4,5-tetrakis(4-carboxyphenyl)benzene; 1,3,5-tris(4′-carboxy[1,1′-biphenyl]-4-yl)benzene; 1,3,5-tris(4-carboxyphenyl)benzene; 2,5-dihydroxyterephthalic acid; 2,6-naphthalenedicarboxylic acid; 2-hydroxyterephthalic acid; 2-methylimidazole; 3,3′,5,5′-tetracarboxydiphenylmethane; 4,4′,4″-s-triazine-2,4,6-triyl-tribenzoic acid; 9,10-anthracenedicarboxylic acid; biphenyl-3,3′,5,5′-tetracarboxylic acid; biphenyl-3,4′,5-tricarboxylic acid; imidazole, terephthalic acid; trimesic acid; [1,1″:4′,1″]terphenyl-3,3′,5,5′-tetracarboxylic acid; or combinations thereof.

Aspect 11: The composition of any one of aspects 1, 2, or 7-9, wherein the metal-organic framework comprises a chromium metal-organic framework, an iron metal-organic framework, or a zirconium metal-organic framework.

Aspect 12: The composition of any one of aspects 1, 2, or 7-9, wherein the metal-organic framework is MIL-101(Cr), PCN-333(Cr), PCN-333(Fe), Tb-mesoMOF, MOF-74-III, PCN-777, PCN-69, Zr-UiO-68, Zr-UIO-67-8F, UiO-68, MOF818, FDM-3, Tb-TATB, MIL-101-4F, or MIL-101-Br(Cr)

Aspect 13: The composition of any one of aspects 1, 2, or 7-9, wherein the metal-organic framework is MIL-101(Cr).

Aspect 14: The composition of any one of aspects 1-13, wherein the at least one nanospace has an average window size less than the size of the frustrated Lewis pair.

Aspect 15: The composition of any one of aspects 1-14, wherein the at least one nanospace has an average window size from 1.0 nm to 3.0 nm.

Aspect 16: The composition of any one of aspects 1-15, wherein the at least one nanospace has an average diameter from 1.5 nm to 6.0 nm.

Aspect 17: The composition of any one of aspects 1-16, wherein the Lewis acid is a compound of Formula I:

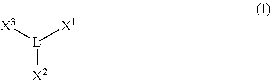

wherein:
L is a group 13 element;
X¹ and X² are each independently selected from alkyl, cycloalkyl, aryl, or heteroaryl, each of which may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl; and
X³ is selected from hydrogen, halogen, alkyl, cycloalkyl, aryl, or heteroaryl, each of which except for hydrogen and halogen may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl.

Aspect 18: The composition of any one of aspects 1-16, wherein the Lewis acid is a compound of Formula II:

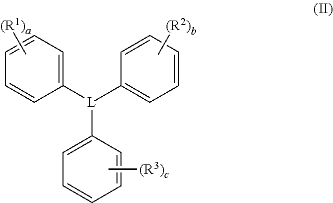

wherein:
L is a group 13 element;
R¹ R², and R³ are independently selected at each occurrence from amino, cyano, halo, hydroxy, nitro, phenyl, C₁₋₆ alkyl, C₃₋₆ cycloalkyl, C₁₋₆ fluoroalkyl, C₁₋₆ chloroalkyl, C₁₋₆ alkoxy, and C₁₋₆ alkylphenyl; and
a, b, and c are independently selected from 0, 1, 2, 3, 4, or 5.

Aspect 19: The composition of any one of claims 1-16, wherein the Lewis acid is a compound of Formula III:

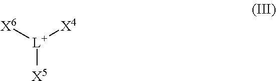

wherein:
L⁺ is a group 13 ion;
X⁴ and X⁵ are each independently selected from alkyl, cycloalkyl, haloalkyl, aryl, or heteroaryl, wherein each X⁴ and X⁵ may be optionally substituted with at least one substituent selected from cyano, halo, hydroxyl, nitro, optionally substituted mesityl, C₁₋₆ alkyl, C₃₋₆ cycloalkyl, C₁₋₆ alkoxy, phenyl, C₁₋₆ alkylphenyl, heterocyclyl, C₁₋₆ alkylheterocyclyl, SO₂aryl, and SO₂alkyl; or
X⁴ and X⁵ are brought together with the atom to which they are attached to form a four to ten atom saturated or unsaturated monocyclic or bicyclic ring which may optionally comprises one or two additional heteroatoms selected from nitrogen, oxygen, and sulfur and which may be optionally substituted with one or more substituents selected from amino, cyano, halo, nitro, trifluoromethyl, C₁₋₆ alkyl, C₁₋₆ alkoxy, —S(C₁₋₆ alkyl), —S(O)(C₁₋₆ alkyl), or —S(O)₂(C₁₋₆ alkyl); and
X⁶ is an electron pair donor ligand coordinated to L⁺.

Aspect 20: The composition of any one of aspects 1-16, wherein the Lewis acid is selected from B(C₆F₃)₃, B(C₆Cl₅)₃, B(C₆F₅)(C₆Cl₅)₂, B(C₆F₅)₂(C₆Cl₅), Al(C₆F₅)₃, B(C₆F₄H)₃, BCl(C₆F₅)₂, [(ⁱPr₂-NHC)(B(2,6-(CH₃)₂C₆H₃)₂)], [(ⁱPr₂-NHC)(B(3,5-(CH₃)₂C₆H₃)₂)]+, or [(ⁱPr₂-NHC)(B(3,5-(CF₃)₂C₆H₃)₂)]⁺, or combinations thereof.

Aspect 21: The composition of any one of aspects 1-16, wherein the Lewis acid is selected from

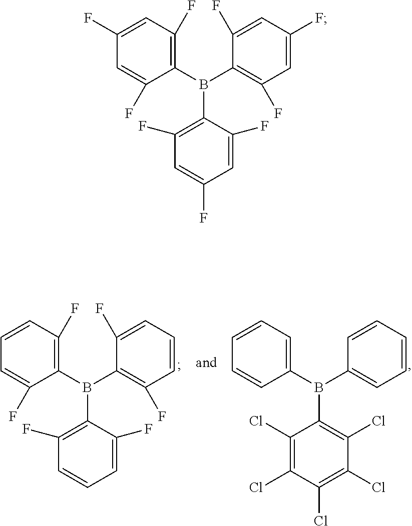 [SMILES: Fc1cc(F)c(B(c2c(F)cc(F)cc2F)c2c(F)cc(F)cc2F)c(F)c1 ; Fc1cccc(F)c1B(c1c(F)cccc1F)c1c(F)cccc1F ; Clc1c(Cl)c(Cl)c(B(c2ccccc2)c2ccccc2)c(Cl)c1Cl]

or combinations thereof.

Aspect 22: The composition of any one of aspects 1-21, wherein the Lewis base is a compound of Formula IV:

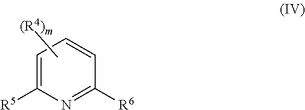

wherein:
R⁴, R⁵, and R⁶ are each independently selected at each occurrence from amino, cyano, halo, nitro, trifluoromethyl, C₁₋₆ alkyl, aryl, C₁₋₆ alkoxy, —S(C₁₋₆ alkyl), —S(O)(C₁₋₆ alkyl), and —S(O)₂(C₁₋₆ alkyl); and
m is 0, 1, 2, or 3.

Aspect 23: The composition of any one of aspects 1-21, wherein the Lewis base is a compound of Formula V:

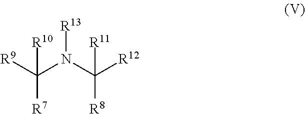

wherein:
R⁷ and R⁸ are each independently optionally substituted C₁₋₆ alkyl; or
R⁷ and R⁸ may be brought together with the atoms to which they are attached to form a five, six, or seven membered heterocyclic ring which may comprise one or two additional heteroatoms selected from nitrogen, oxygen or sulfur and which may be optionally substituted with one or more substituent groups selected from amino, cyano, halo, nitro, trifluoromethyl, C₁₋₆ alkyl, C₁₋₆ alkoxy, —S(C₁₋₆ alkyl), —S(O)(C₁₋₆ alkyl), or —S(O)₂(C₁₋₆ alkyl);
R⁹, R¹⁰, R¹¹, and R¹² are selected from hydrogen, alkyl, cycloalkyl, aryl, or heteroaryl, each which may be optionally substituted with one or more substituents selected form amino, cyano, halo, nitro, trifluoromethyl, C₁₋₆ alkyl, C₁₋₆ alkoxy, —S(C₁₋₆ alkyl), —S(O)(C₁₋₆ alkyl), or —S(O)₂(C₁₋₆ alkyl); and
R¹³ is hydrogen, C₁₋₃ alkyl, aryl, or heteroaryl.

Aspect 24: The composition of any one of aspects 1-21, wherein the Lewis base is a compound of Formula VI:

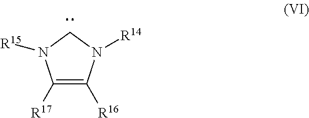

wherein:
R¹⁴ and R¹⁵ are each selected from hydrogen or C₁₋₆ alkyl; and
R¹⁶ and R¹⁷ are each selected from amino, cyano, halo, hydrogen, nitro, trifluoromethyl, C₁₋₆ alkyl, C₁₋₆ alkoxy, NH(C₁₋₆ alkyl), N(independently C₁₋₆ alkyl)₂, —S(C₁₋₆ alkyl), —S(O)(C₁₋₆ alkyl), —S(O)₂(C₁₋₆ alkyl), or phenyl.

Aspect 25: The composition of any one of aspects 1-21, wherein the Lewis base is a compound of Formula VII:

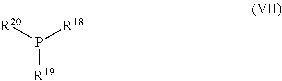

wherein R¹⁸, R¹⁹, and R²⁰ are each independently selected from alkyl, cycloalkyl, aryl, heterocyclyl, and heteroaryl, each of which may be optionally substituted with one or more substituent selected from amino, aryl, cyano, halo, heteorcyclyl, nitro, C₁₋₆ alkyl, or C₁₋₆ alkoxy.

Aspect 26: The composition of any one of aspects 1-21, wherein the Lewis base is a compound of Formula VIII:

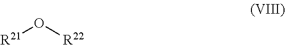

wherein:
R²¹ and R²² are independently selected from alkyl, cycloalkyl, aryl, heterocyclyl, or heteroaryl, each of which is optionally substituted with amino, aryl, cyano, halo, heterocyclyl, nitro, C₁₋₆ alkyl, or C₁₋₆ alkoxy; or
R²¹ and R²² may be brought together with the oxygen to which they are attached to form a four to seven membered saturated or unsaturated ring which may optionally comprise one or two additional heteroatoms selected from nitrogen, oxygen, or sulfur and which may be optionally substituted with one or more substituents selected from amino, cyano, halo, nitro, trifluoromethyl, C₁₋₆ alkyl, C₁₋₆ alkoxy, —S(C₁₋₆ alkyl), —S(O)(C₁₋₆ alkyl), and —S(O)₂(C₁₋₆ alkyl).

Aspect 27: The composition of any one of aspects 1-21, wherein the Lewis base is a compound of Formula IX:

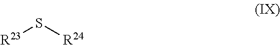

wherein:
R²³ and R²⁴ are independently selected from alkyl, cycloalkyl, aryl, heterocyclyl, or heteroaryl, each of which is optionally substituted with amino, aryl, cyano, halo, heterocyclyl, nitro, C₁₋₆ alkyl, or C₁₋₆ alkoxy; or
R²³ and R²⁴ may be brought together with the sulfur to which they are attached to form a four to seven membered saturated or unsaturated ring which may optionally comprise one or two additional heteroatoms selected from nitrogen, oxygen, or sulfur and which may be optionally substituted with one or more substituents selected from amino, cyano, halo, nitro, trifluoromethyl, C₁₋₆ alkyl, C₁₋₆ alkoxy, —S(C₁₋₆ alkyl), —S(O)(C₁₋₆ alkyl), and —S(O)₂(C₁₋₆ alkyl).

Aspect 28: The composition of any one of aspects 1-21, wherein the Lewis base comprises a bridging heterocyclic compound.

Aspect 29: The composition of any one of aspects 1-21, wherein the Lewis base is selected from:

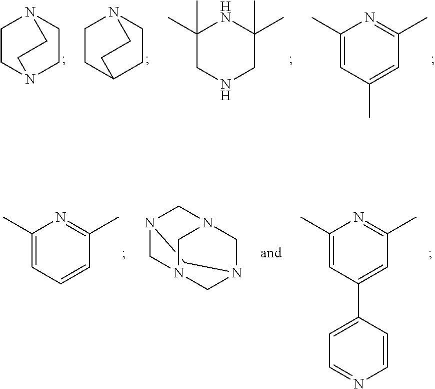

or any combination thereof.

Aspect 30: The composition of any one of aspects 1-21, wherein the Lewis base is selected from:

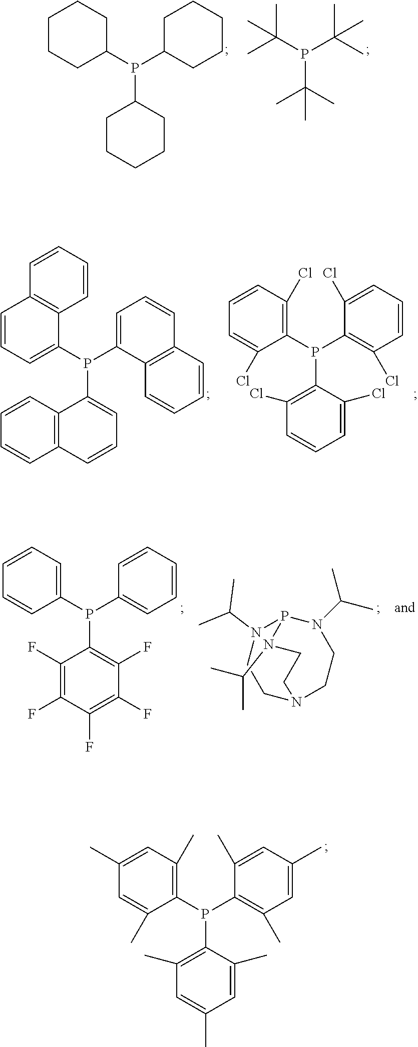

or combinations thereof.

Aspect 31: The composition of any one of aspects 1-21, wherein the Lewis base is selected from diethyl ether, 1,4,-dioxane, tetrahydrofuran, and tetrahydropyran, or combinations thereof.

Aspect 32: The composition of any one of aspects 1-16, wherein the Lewis acid and Lewis base of the frustrated Lewis pair a covalently linked by a divalent organic linker.

Aspect 33: The composition of aspect 32, wherein the frustrated Lewis pair is selected from:

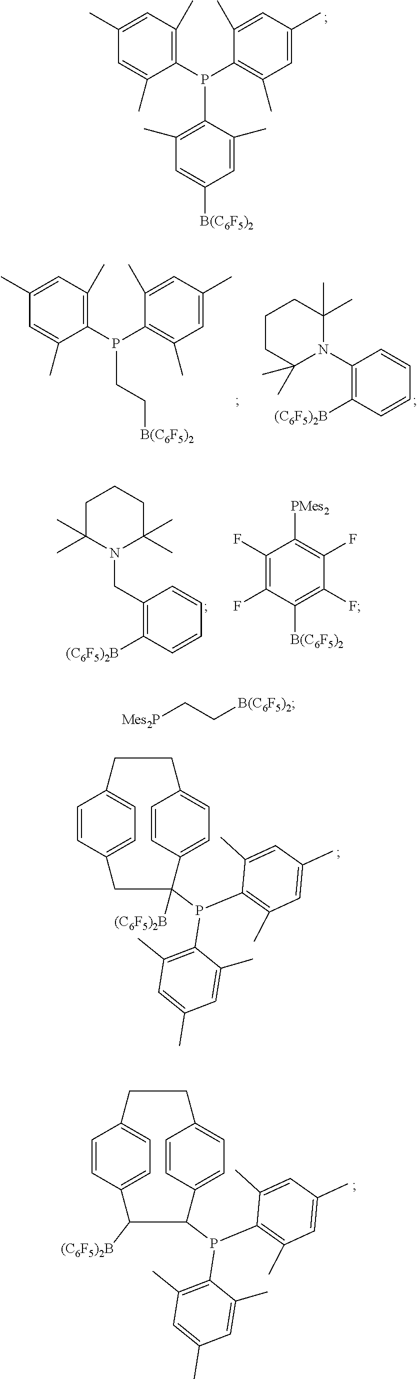

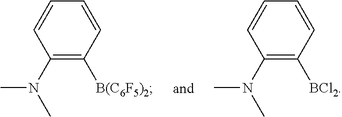

Aspect 34: The composition of any one of aspects 1-16, wherein the metal-organic framework comprises a chromium metal-organic framework, an iron metal-organic framework, or a zirconium metal-organic framework, the Lewis base comprises a heterobicyclo-compound, a heterotricyclo-compound, or a heterotetracyclo-compound, and the Lewis acid is a compound of Formula I:

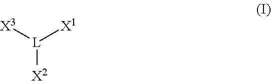

wherein:
L is B or Al;
X¹ and X² are each independently selected from alkyl, cycloalkyl, aryl, or heteroaryl, each of which may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl; and
X³ is selected from hydrogen, halogen, alkyl, cycloalkyl, aryl, or heteroaryl, each of which except for hydrogen and halogen may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl.

Aspect 35: The composition of aspect 34, wherein the Lewis base comprises a heterobicyclo-compound, and the Lewis acid is a compound of Formula II:

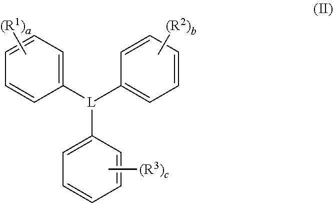

wherein:
L is B or Al;
R¹ R², and R³ are independently selected at each occurrence from amino, cyano, halo, hydroxy, nitro, phenyl, C₁₋₆ alkyl, C₃₋₆ cycloalkyl, C₁₋₆ fluoroalkyl, C₁₋₆ chloroalkyl, C₁₋₆ alkoxy, and C₁₋₆ alkylphenyl; and
a, b, and c are independently selected from 0, 1, 2, 3, 4, or 5.

Aspect 36: The composition of aspect 35, wherein L is B and each R¹ R², and R³ is halo.

Aspect 37: The composition of aspect 1, wherein the metal organic framework is MOF818, FDM-3, Tb-TATB, Zr-UIO-68, Zr-UIO-67-8F, PCN-333(Fe), PCN-333(Cr), MIL-101(Cr), MIL-101-4F, or MIL-101-Br(Cr), the Lewis acid is B(C₆F₅)₃, and the Lewis base is 1,4-diazabicyclo[2.2.2]octane (DABCO).

Aspect 38: The composition of aspect 1, wherein the metal organic framework is MOF818, FDM-3, Tb-TATB, Zr-UIO-68, Zr-UIO-67-8F, PCN-333(Fe), PCN-333(Cr), MIL-101(Cr), MIL-101-4F, or MIL-101-Br(Cr), the Lewis acid is B(C₆F₅)₃, and the Lewis base is hexamethylenetetramine (HMTA).

Aspect 39: The composition of aspect 1, wherein the metal organic framework is MOF818, FDM-3, Tb-TATB, Zr-UIO-68, Zr-UIO-67-8F, PCN-333(Fe), PCN-333(Cr), MIL-101(Cr), MIL-101-4F, or MIL-101-Br(Cr), the Lewis acid is MesB(C₆F₅)₃, and the Lewis base is 1,4-diazabicyclo[2.2.2]octane (DABCO).

Aspect 40: The composition of aspect 1, wherein the metal organic framework is MOF818, FDM-3, Tb-TATB, Zr-UIO-68, Zr-UIO-67-8F, PCN-333(Fe), PCN-333(Cr), MIL-101(Cr), MIL-101-4F, or MIL-101-Br(Cr), the Lewis acid is MesB(C₆F₅)₃, and the Lewis base is hexamethylenetetramine (HMTA).

Aspect 41: A composition comprising:
a porous metal-organic framework (MOF) having at least one nanospace and comprising at least one or more metal ions and one or more organic linker ligands; and
a frustrated Lewis pair comprising a Lewis base and a Lewis acid;
wherein the Lewis base and/or Lewis acid of the frustrated Lewis pair is covalently bound to at least one or more organic linker ligands; and
wherein the frustrated Lewis pair is contained within at least one nanoscopic cage of the metal-organic framework.

Aspect 42: The composition of aspect 41, wherein the Lewis acid of the frustrated Lewis pair is covalently bound to at least one or more organic linker ligands.

Aspect 43: The composition of aspect 41, wherein the Lewis base of the frustrated Lewis pair is covalently bound to at least one or more organic linker ligands.

Aspect 44: The composition of aspect 43, wherein the one or more organic linker ligands are selected from:

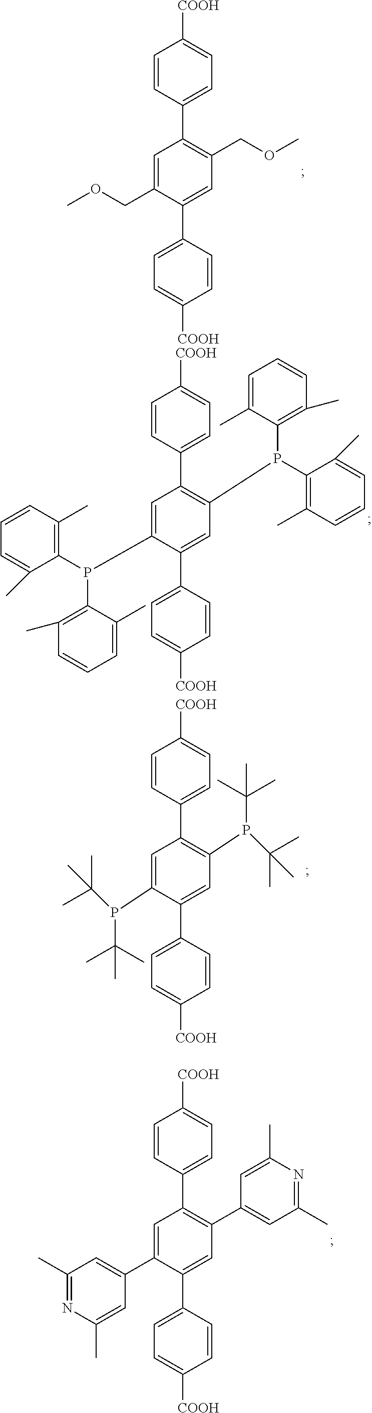

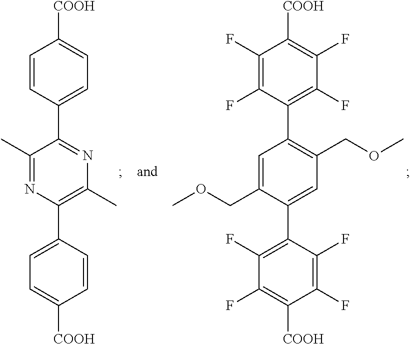

or combinations thereof.

Aspect 45: The composition of any one of aspects 41-44, wherein the one or more metal ions are an ion of a metal selected from Mg, Ca, Sr, Ba, Sc, Y, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Hg, Al, Ga, In, Tl, Si, Ge, Sn, Pb, As, Sb, and Bi, or combinations thereof.

Aspect 46: The composition of any one of aspects 41 and 43-45, wherein the Lewis acid is a compound of Formula I:

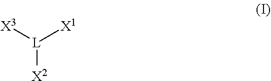

wherein:
L is a group 13 element;
X¹ and X² are each independently selected from alkyl, cycloalkyl, aryl, or heteroaryl, each of which may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl; and
X³ is selected from hydrogen, halogen, alkyl, cycloalkyl, aryl, or heteroaryl, each of which except for hydrogen and halogen may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl.

Aspect 47: The composition of any one of aspects 41 and 43-45, wherein the Lewis acid is a compound of Formula II:

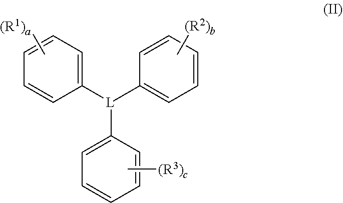

wherein:
L is a group 13 element;
R¹ R², and R³ are independently selected at each occurrence from amino, cyano, halo, hydroxy, nitro, phenyl, C₁₋₆ alkyl, C₃₋₆ cycloalkyl, C₁₋₆ fluoroalkyl, C₁₋₆ chloroalkyl, C₁₋₆ alkoxy, and C₁₋₆ alkylphenyl; and
a, b, and c are independently selected from 0, 1, 2, 3, 4, or 5.

Aspect 48: The composition of any one of aspects 41 and 43-45, wherein the Lewis acid is a compound of Formula III:

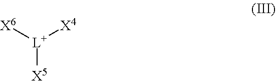

wherein:
L⁺ is a group 13 ion;
X⁴ and X⁵ are each independently selected from alkyl, cycloalkyl, haloalkyl, aryl, or heteroaryl, wherein each X⁴ and X⁵ may be optionally substituted with at least one substituent selected from cyano, halo, hydroxyl, nitro, optionally substituted mesityl, C₁₋₆ alkyl, C₃₋₆ cycloalkyl, C₁₋₆ alkoxy, phenyl, C₁₋₆ alkylphenyl, heterocyclyl, C₁₋₆ alkylheterocyclyl, SO₂aryl, and SO₂alkyl; or
X⁴ and X⁵ are brought together with the atom to which they are attached to form a four to ten atom saturated or unsaturated monocyclic or bicyclic ring which may optionally comprises one or two additional heteroatoms selected from nitrogen, oxygen, and sulfur and which may be optionally substituted with one or more substituents selected from amino, cyano, halo, nitro, trifluoromethyl, C₁₋₆ alkyl, C₁₋₆ alkoxy, —S(C₁₋₆ alkyl), —S(O)(C₁₋₆ alkyl), or —S(O)₂(C₁₋₆ alkyl); and
X⁶ is an electron pair donor ligand coordinated to L⁺.

Aspect 49: The composition of any one of aspects 41 and 43-45, wherein the Lewis acid is selected from B(C₆F₃)₃, B(C₆Cl₅)₃, B(C₆F₅)(C₆Cl₅)₂, B(C₆F₅)₂(C₆Cl₅), Al(C₆F₅)₃, B(C₆F₄H)₃, BCl(C₆F₅)₂, [(ⁱPr₂-NHC)(B(2,6-(CH₃)₂C₆H₃)₂)], [(ⁱPr₂-NHC)(B(3,5-(CH₃)₂C₆H₃)₂)]⁺, or [(ⁱPr₂- NHC)(B(3,5-(CF₃)₂C₆H₃)₂)]+, or combinations thereof.

Aspect 50: The composition of any one of aspects 41 and 43-45, wherein the Lewis acid is selected from

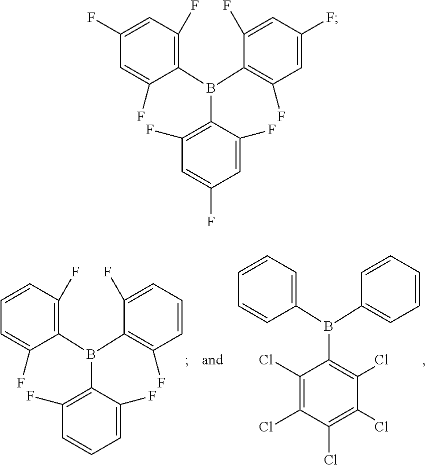 [SMILES: Fc1cc(F)c(B(c2c(F)cc(F)cc2F)c2c(F)cc(F)cc2F)c(F)c1 ; Fc1cccc(F)c1B(c1c(F)cccc1F)c1c(F)cccc1F ; Clc1c(Cl)c(Cl)c(B(c2ccccc2)c2ccccc2)c(Cl)c1Cl]

or combinations thereof.

Aspect 51: A composition comprising:
a porous metal-organic framework (MOF) having at least nanospace and comprising one or more metal ions and one or more chiral organic linker ligands; and
a frustrated Lewis pair comprising a Lewis base and a Lewis acid;
wherein the frustrated Lewis pair is contained within at least one nanospace of the metal-organic framework.

Aspect 52: The composition of aspect 51, wherein the Lewis acid and/or Lewis base are covalently bound to at least one of the one or more organic linker ligands.

Aspect 53: The composition of any one of aspects 51 or 52, wherein the chiral organic linker ligand is selected from:

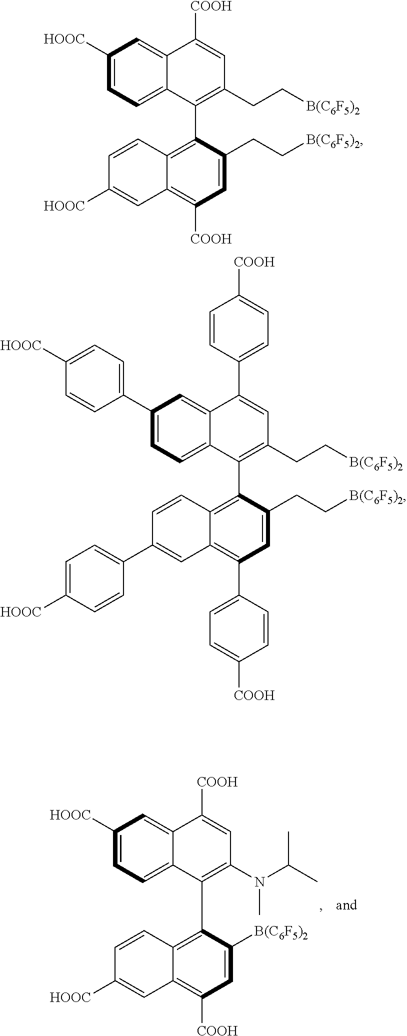

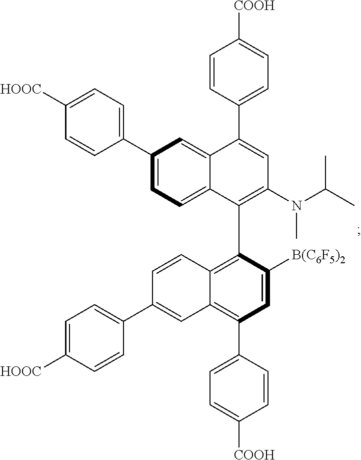

or combinations thereof.

Aspect 54: The composition of any one of aspects 51-53, wherein the one or more metal ions are an ion of a metal selected from Mg, Ca, Sr, Ba, Sc, Y, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Hg, Al, Ga, In, TI, Si, Ge, Sn, Pb, As, Sb, and Bi, or combinations thereof.

Aspect 55: A composition produced by the process comprising:
(a) contacting a porous material with a Lewis base, wherein the porous material comprises a porous metal-organic framework (MOF) or a porous organic polymer (POP) to produce a first porous material; and
(b) contacting the first porous material with a Lewis acid to produce a frustrated Lewis pair.

Aspect 56: The composition of aspect 55, wherein the metal-organic framework comprises a chromium metal-organic framework, an iron metal-organic framework, or a zirconium metal-organic framework, the Lewis base comprises a heterobicyclo-compound, a heterotricyclo-compound, or a heterotetracyclo-compound, and the Lewis acid is a compound of Formula I:

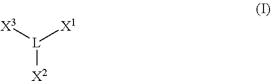

wherein:
L is B or Al;
X¹ and X² are each independently selected from alkyl, cycloalkyl, aryl, or heteroaryl, each of which may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SOzaryl, or SOzalkyl; and
X³ is selected from hydrogen, halogen, alkyl, cycloalkyl, aryl, or heteroaryl, each of which except for hydrogen and halogen may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl.

Aspect 57: The composition of aspect 56, wherein the Lewis base comprises a heterobicyclo-compound, and the Lewis acid is a compound of Formula II:

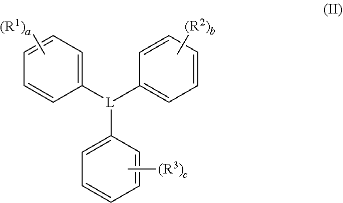

wherein:
L is B or Al;
R¹ R², and R³ are independently selected at each occurrence from amino, cyano, halo, hydroxy, nitro, phenyl, C₁₋₆ alkyl, C₃₋₆ cycloalkyl, C₁₋₆ fluoroalkyl, C₁₋₆ chloroalkyl, C₁₋₆ alkoxy, and C₁₋₆ alkylphenyl; and
a, b, and c are independently selected from 0, 1, 2, 3, 4, or 5.

Aspect 58: The composition of aspect 57, wherein L is B and each R¹ R², and R³ is halo.

Aspect 59: A composition produced by the process comprising:
providing a porous metal-organic framework (MOF) having at least one nanospace and comprising at least one or more metal ions and one or more organic linker ligands;
contacting the metal-organic framework with a Lewis base such that the Lewis base is contained within at least one nanospace of the metal-organic framework to form a MOF-Lewis base adduct; and
contacting the MOF-Lewis base adduct with a Lewis acid such that the Lewis base and Lewis acid form a frustrated Lewis pair within the nanospace of the metal-organic framework.

Aspect 60: The process of aspect 59, wherein the Lewis base is contained within the nanospace of the metal-organic framework by coordinating to at least one metal ion.

Aspect 61: A composition produced by the process comprising comprising:
providing a porous metal-organic framework having at least one nanospace;
contacting the metal organic framework with a Lewis base precursor such that the Lewis base precursor is contained within the at least one nanospace of the metal-organic framework to from a Lewis base precursor-MOF adduct; and
contacting the Lewis base precursor-MOF adduct with a Lewis acid precursor such that the Lewis base precursor and Lewis acid precursor react to form the frustrated Lewis pair within the nanospace of the metal-organic framework.

Aspect 62: The process of aspect 61, wherein the Lewis base precursor and the Lewis acid precursor react to form the frustrated Lewis pair by forming a covalent bond.

Aspect 63: The composition of any one of aspects 1-59, wherein the frustrated Lewis pair is in the amount of from about 0.1 mmol to about 5 mmol per 1 g of porous material.

Aspect 64: The composition of any one of aspects 1-59, wherein the outer surface of the porous material is functionalized with perfluoroalkyl moieties.

Aspect 65: The composition of aspect 64, wherein the perfluoroalkyl moieties comprise from 7 to 20 carbon atoms.

Aspect 66: The composition of any one of aspects 1-65, wherein the composition has a gravimetric capacity of about 2.0 wt % to about 10.0 wt % and volumetric capacity of about 0.015 kg H₂/L to about 0.100 kg H₂/L under hydrogen pressure of less than or equal 200 bar at ambient temperature.

Aspect 67: A fuel cell comprising the composition of any one of aspects 1-66.

Aspect 68: A process for hydrogenation or hydrogenolysis of an organic compound with at least one point of unsaturation, comprising:
contacting the organic compound with a composition of any one of aspects 1-66 in the presence of hydrogen gas.

Aspect 69: The process of aspect 68, wherein the at least one point of unsaturation is an imino group, and wherein the imino group is converted into an amino group.

Aspect 70: The process of aspect 68, wherein the at least one point of unsaturation is an α,β unsaturated imino group, wherein the imino group is converted into an amino group.

Aspect 71: The process of aspect 68, wherein the at least one point of unsaturation is an alkenyl group, and wherein the alkenyl group is converted into an alkyl group.

Aspect 72: The process of aspect 68, wherein the at least one point of unsaturation is an alkynyl group, and wherein the alkynyl group is converted into an alkenyl group or an alkyl group.

Aspect 72: The process of aspect 68, wherein the at least one point of unsaturation is a carbonyl group selected from an aldehyde or ketone, and wherein the carbonyl group is converted into an alcohol via hydrogenation.

Aspect 73: The process of aspect 68, wherein the at least one point of unsaturation is a carbonyl group selected from an aldehyde or a ketone, and wherein the carbonyl group is converted into a methylene group via hydrogenolysis.

Aspect 74: The process of aspect 68, wherein the at least one point of unsaturation is an oxide group, and wherein the oxime group is converted into a hydroxyamine group of an alkoxyamine group.

### EXAMPLES

### Example 1. Synthesis and Hydrogenation Activity of MIL-101(Cr)-LP

Introduction of Lewis Pair into MIL-101(Cr) Metal-Organic Framework

The base moiety of the frustrated Lewis pair is first anchored to the open metal sites within the metal-organic framework through coordination interaction, which is followed by the introduction of the corresponding acid moiety of the frustrated Lewis pair. Given the strong coordination interaction, it is anticipated that the frustrated Lewis pair would be stabilized within the metal-organic framework yet accessible to substrates.

As a proof of concept, dehydrated MIL-101(Cr), Cr₃(OH)O(BDC)₃ (BDC=1,4-benzenedicarboxylate), was selected to anchor the frustrated Lewis pair considering its large pores or cages, abundant open metal sites upon activation, and high stability; the frustrated Lewis pair, composed of B(C₆F₅)₃ and 1,4-diazabicyclo[2.2.2]octane (DABCO) that features one potential coordination site, is used as a model for grafting into the metal-organic framework. This frustrated Lewis pair reacts not only with neutral borane pinacolborane (HBPin) to afford a borenium salt capable of reducing imine compounds but also with H₂ gas to catalyze the hydrogenation of alkylidenemalonates as a frustrated Lewis pair (FLP). This process includes the stepwise grafting of DABCO and B(C₆F₅)₃ into MIL-101 (Cr) and the resultant MIL-101 (Cr) anchored with B(C₆F₅)₃/DABCO pair is named MIL-101(Cr)-LP. With the frustrated Lewis pair anchored and stabilized within the nanospace of the metal-organic framework, MIL-101(Cr)-LP can efficiently catalyze both the imine reduction with interesting size and steric selectivity and the hydrogenation of alkylidene malonates directly by using H₂.

MIL-101(Cr) was synthesized and activated according to the literature. MIL-101(Cr)-LP was prepared through a stepwise method: the activated MIL-101(Cr) was soaked into a Lewis base toluene solution, then washed, filtered, and dried under vacuum; subsequently, the solution of B(C₆F₅)₃ (Lewis acid) was added to the sample, stirred for several hours, and then washed with fresh toluene several times until no frustrated Lewis pair traces could be detected in the filtrate by liquid nuclear magnetic resonance (NMR) measurement. This process excludes the influence of the adsorbed free frustrated Lewis pair molecules in the pores of the framework. Since the theoretical maximum loading amount of anchored LP on MIL-101(Cr) is 1.47 mmol LP per 1 g MIL-101(Cr), MIL-101(Cr)-LP (1 mmol LP per 1 g MIL-101(Cr) or 0.34 LP per unsaturated Cr site) was used in the following characterization.

### Structural Characterization of MIL-101(Cr)-LP

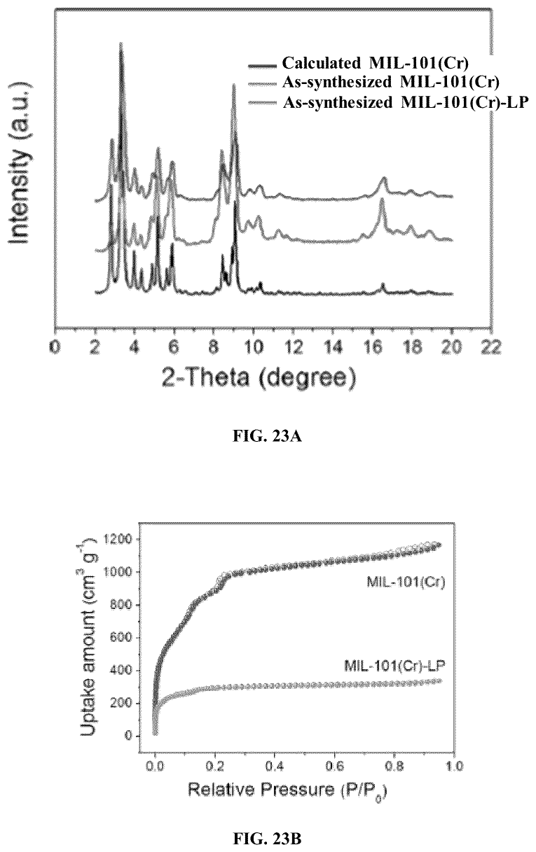

The phase purity of MIL-101(Cr)-LP was verified through powder X-ray diffraction (PXRD)measurements, and the porosity of the materials was studied by N₂ gas sorption at 77 K. As shown in FIG. 23A, the PXRD patterns of MIL-101(Cr)-LP are consistent with the calculated ones and those of the pristine MIL-101(Cr), indicating the preservation of the framework structure during the loading process. The N₂ sorption studies (FIG. 23B) reveal that, in comparison with MIL-101, there is an obvious decrease in the Brunauer-Emmett-Teller surface area (from 2,724 to 1,013 m²/g) and reduction in pore sizes (Figures S6 and S7) for MIL-101(Cr)-LP because the frustrated Lewis pair molecules are grafted onto the pores of MIL-101(Cr) (FIG. 23B).

### Characterization of Anchored LP in MIL-101(Cr)-LP

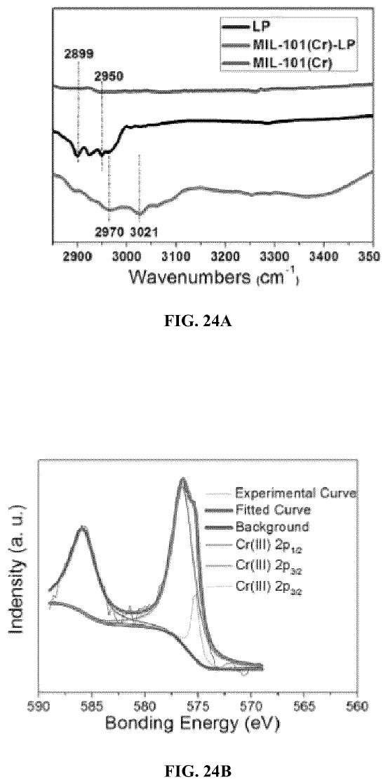

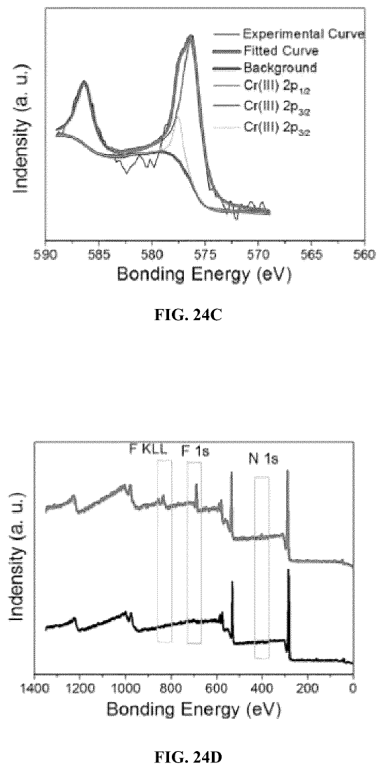

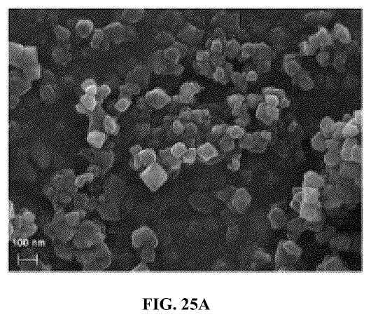

The combination of a frustrated Lewis pair and MIL-101 was confirmed by Fourier transform infrared spectroscopy (FTIR) analysis. The spectrum of MIL-101(Cr)-LP exhibits characteristic peaks from the frustrated Lewis pair. As shown in FIG. 24A, the presence of new bands at high wavenumbers (2,800-3,200 cm⁻¹) is due to the aliphatic C—H stretching vibrations of the frustrated Lewis pair. Some obvious shifts are observed between MIL-101(Cr)-LP and the original frustrated Lewis pair complex (from 2,899 and 2,950 to 2,970 and 3,021 cm⁻¹), indicating a coordination interaction between the N atom of the frustrated Lewis pair and the metal site of MIL-101(Cr).53,54 The characteristic frustrated Lewis pair band at 1,455 cm⁻¹ is slightly shifted to 1,463 cm⁻¹ after the frustrated Lewis pair is loaded within the metal-organic framework (Figures S10 and S11), further suggesting possible coordination interaction between the frustrated Lewis pair and the MIL-101 framework.

X-ray photoelectron spectroscopy (XPS) spectra of MIL-101(Cr) and MIL-101(Cr)-LP were used to further verify the coordination interaction between the frustrated Lewis pair and the Cr open metal sites in MIL-101(Cr). As shown in FIGS. 24B and 24C, the Cr(2p) spectra of MIL-101(Cr)-LP are obviously different from the Cr(2p) spectra of MIL-101(Cr). The Cr (2p_{1/2}) and Cr (2p_{3/2}) peaks of MIL-101(Cr)-LP are shifted by about 0.58-2 eV toward higher binding energies, compared with those of MIL-101(Cr). Such shifts indicate an increase in the electron density of Cr(III), which can be attributed to the interaction between Cr and DABCO. The survey spectra of MIL-101(Cr)-LP indicate the existence of the F and N elements of the frustrated Lewis pair molecule within the metal-organic framework (FIG. 24D).

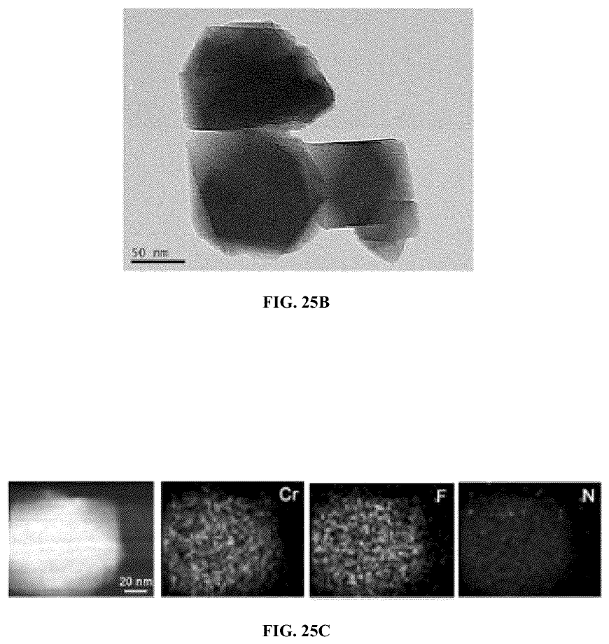

Scanning electron microscopy (SEM) and transmission electron microscopy (TEM) were used to investigate the morphology of MIL-101(Cr)-LP. As shown in FIGS. 25A and 25B, the SEM and TEM images exhibited regular octahedral crystals of MIL-101(Cr)-LP with an average diameter of ˜100 nm. For investigating the distribution of frustrated Lewis pair in MIL-101(Cr)-LP, high-angle annular dark-field scanning TEM (HAADF-STEM), and energy dispersive X-ray spectroscopy (EDS) elemental mapping analyses were used. As presented in FIG. 25C, the Cr, F, and N elements are evenly distributed inside the octahedral crystal of MIL-101(Cr)-LP, suggesting the integration of frustrated Lewis pair inside the pores of the metal-organic framework. Furthermore, SEM and EDS elemental mapping analyses also revealed an even distribution of B, F, and N in large scale. These results indicated that the frustrated Lewis pair was homogeneously distributed in the MIL-101(Cr) pores without the presence of accumulation in the particular regions.

### Methods for Characterizing Hydrogen Adsorption of Disclosed Compositions

The hydrogen adsorption capacity of the compositions disclosed herein can be determined using the following methods. The hydrogen adsorption is taken for frustrated Lewis pair-impregnated metal-organic frameworks using a Sievert isotherm apparatus. The high-precision hydrogen uptake at the different equilibrium pressures up to 100 bars is measured at 298 K and 273 K or lower using well-controlled temperature jackets at the sample cell and Sievert isotherm housing. The hydrogen pressure is calibrated against gas density calculated from GASPAK and both excess and absolute hydrogen storage capacities are reported. The hydrogen adsorption enthalpy is derived from the hydrogen uptakes under different temperatures but the same pressure using Clausius-Clapeyron equation.

Structural and mechanistic studies of H₂ adsorption in frustrated Lewis pair-impregnated metal-organic frameworks can be carried out using conventional and advanced characterization techniques. For example, single crystal and powder X-ray diffraction can be used to first confirm the metal-organic framework structure. TEM and SEM can be used to study the adsorbent morphology. Combined IR, NMR, and elemental analysis are conducted to characterize and quantify the loading amount of frustrated Lewis pair in metal-organic framework. The surface areas and pore size distributions of the frustrated Lewis pair-impregnated metal-organic framework can be examined through nitrogen adsorption at 77 K using a Surface Area Analyzer. Low-energy ion scattering spectroscopy can be applied for direct mapping of hydrogen on surfaces, in situ neutron diffraction and operando NMR can be used to study the site for adsorbed hydrogen and in situ x-ray pair-distribution function (PDF) and small angle x-ray scattering (SAXS) can be used to study the adsorbent structural changes under the elevated hydrogen pressure during adsorption.

### Effect of LP Loading on Catalysis Performance

Compared with the expensive and potentially toxic metal-based catalyst, the metal-free frustrated Lewis pairs are shown to have enhanced catalytic performance in the synthesis of B-containing organic compounds. To examine the catalytic performances of frustrated Lewis pair loaded MIL-101(Cr), we prepared MIL-101(Cr)-LP-1(0.5 mmol LP per 1 g MIL-101(Cr) or 0.17 LP per unsaturated Cr site) and MIL-101(Cr)-LP-2 (0.75 mmol LP per 1 gMIL-101(Cr) or 0.26 LP per unsaturated Cr site) to investigate the optimal uptake amount for catalyzing the imine reduction reactions. The loading amount of frustrated Lewis pair in MIL-101(Cr)-LP was quantified with 1H NMR by decomposing MIL-101(Cr)-LP in 5 wt % NaOH D₂O solution and later confirmed by elemental analysis. The pore size of these catalysts was studied through N₂ isotherms at 77 K. The catalytic performances of the MIL-101(Cr)-LP catalysts were evaluated by exposing N-tert-butyl-1-phenylmethanimine to 1.2 equiv of HBPin and 20 mg catalyst in toluene to result in the reduction to the corresponding pinacolboramide after 2 hr. As shown in Table 1, the lowest frustrated Lewis pair uptake amount sample, MIL-101(Cr)-LP-1, gave 83% yield, whereas MIL-101(Cr)-LP exhibited full conversion. Therefore, MIL-101(Cr)-LP was chosen for subsequent studies. The control experiment using pristine MIL-101(Cr) was conducted, but no reduction product was detected in the reaction solvent even after 48 hr, meaning that MIL-101 is inactive for the imine reduction reaction.

TABLE 1
Investigation of MIL-101(Cr) and MIL-101(Cr)-LP in the Catalytic
Reduction of Imine

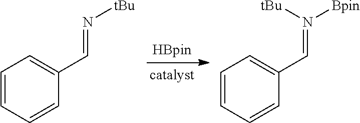

| Entry | Catalyst | Yield^{a} |
|---|---|---|
| 1 | MIL-101(Cr) | 0%^{b} |
| 2 | MIL-101(Cr)-LP-1 | 83% |
| 3 | MIL-101(Cr)-LP-2 | 91% |
| 4 | MIL-101(Cr)-LP | 100% |

Reaction conditions: 20 mg catalyst, 55 mg (0.43 mmol) HBpin, 56 mg (0.35 mmol) imine, 3 mL toluene, room temperature, 2 hr.
^{a}Yields were determined by liquid NMR.
^{b}Reaction time: 48 hr.

### Catalysis Studies for Reduction of Different Imine Compounds

Imine compounds with different substituting groups were used to investigate the difference in catalytic properties between the heterogeneous MIL-101(Cr)-LP and its homogeneous frustrated Lewis pair counterpart. As shown in Table 2, the reaction yields catalyzed by MIL-101(Cr)-LP from related imine compounds are 38% for N-benzylideneaniline (Table 2, Entry 1), 100% for N-benzylidene-1-phenylmethanamine (Table 2, Entry 2), and 22% for acridine (Table 2, Entry 3). For comparison, the yields are 87%, 85%, and 91% catalyzed by 3.5 mol % homogeneous frustrated Lewis pair catalyst, respectively. The comparison of the yields of these products reveals a very interesting phenomenon: the reduction product yields of Table 1, Entry 4 and Table 2, Entry 2 are similar or even higher than the homogeneous frustrated Lewis pair catalyst, which clearly indicates that MIL-101(Cr)-LP has comparable performance with the homogeneous frustrated Lewis pair for catalytic imine reduction with HBPin at the same conditions; however, the reduction product yields of Table 2, Entry 1 and Entry 3 are much less than the homogeneous frustrated Lewis pair catalyst. The catalysis results reveal that the steric effect close to the N atoms shows more obvious selectivity in MIL-101(Cr)-LP than the frustrated Lewis pair homogeneous catalyst. This kind of steric selectivity could presumably be due to the confinement effect imparted by the porous metal-organic framework structure, which restricts the accessibility of buried C═N double bonds to the frustrated Lewis pair active centers that are anchored on the pore walls of metal-organic framework.

With the increase in the size of the imine substrate, the reduction reaction yield decreased. The reaction yield catalyzed by homogeneous frustrated Lewis pair for N-benzhydryl-1-phenylmethanimine (Table 2, Entry 4) and N-(diphenylmethylene)-1,1-diphenylmethanamine (Table 2, Entry 5) are 60% and 29%, respectively. However, the reaction yield for Table 2, Entry 4 is 42% and none of the product could be observed in the heterogeneous MIL-101(Cr)-LP-catalyzed reaction for Table 2, Entry 5 even after 24 hr. The high size selectivity performance can be attributed to the window structure in MIL-101(Cr)-LP. Considering the existence of the coordinated frustrated Lewis pair on the window, the size of the window (with diameter of ˜1.0 nm) could be smaller than the diameter of the molecule in Table 2, Entry 5 (with diameter of 1.3 nm), thus the large imine molecule could not enter into the pore of MIL-101(Cr)-LP.

TABLE 2
Catalysis Studies for the Reduction of C═N Double Bonds

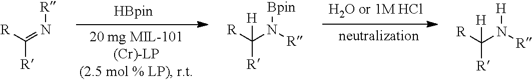

| | | Yield | Yield |
|---|---|---|---|
| Entry | Substrate | (FLP@MOF) | (FLP)^{a} |
| 1 | 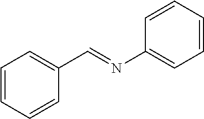 [SMILES: C(=N\c1ccccc1)/c1ccccc1] | 38%^{b} | 87%^{b} |
| 2 | 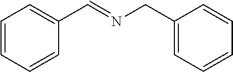 [SMILES: C(=N\Cc1ccccc1)/c1ccccc1] | 100%^{b} | 85%^{b} |
| 3 | 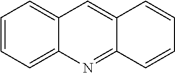 [SMILES: c1ccc2nc3ccccc3cc2c1] | 22%^{b} | 91%^{b} |
| 4 | 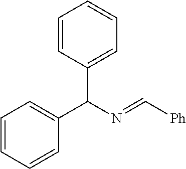 [SMILES: C(=N\C(c1ccccc1)c1ccccc1)/c1ccccc1] | 42%^{c} | 60%^{c} |
| 5 | 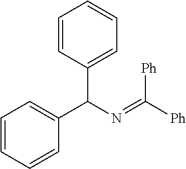 [SMILES: c1ccc(C(=NC(c2ccccc2)c2ccccc2)c2ccccc2)cc1] | 0%^{c} | 29%^{c} |

Reaction conditions: 20 mg MIL-101(Cr)-LP (3.5 mol % LP) for heterogeneous of 3.5 mol % LP for homogenous catalytic reaction, 55 mg (0.43 mmol) HBpin, 0.35 mmol substrate, 3 mL toluene
^{a}Yield is relative to substrate
^{b}Reaction time: 3 hr.
^{c}Reaction time: 24 hr.

### Catalysis Studies for Hydrogenation of Alkylidene Malonate Compounds

frustrated Lewis pairs show remarkable reactivity toward the activation of molecular H₂. However, the frustrated Lewis pair-promoted catalytic hydrogenation of electron-poor unsaturated compounds still presents a significant challenge. In view of the interesting catalytic performance of MIL-101(Cr)-LP in the above imine reduction reactions, MIL-101(Cr)-LP was used to achieve the catalytic hydrogenation of alkylidene malonates, which is one important kind of electron-poor unsaturated compound, directly using H₂ gas. Alkylidene malonate compounds with different substituted groups were used to investigate the catalytic performance of the heterogeneous MIL-101(Cr)-LP. As presented in Table 3, the reaction yields catalyzed by MIL-101(Cr)-LP from related alkylidene malonate compounds are 95% for diethyl 2-benzylidenemalonate (Entry 1), 84% for diethyl 2-(2-methylpropylidene)malonate (Entry 2), 83% for diethyl 2-hexylidenemalonate (Entry 3), and 88% for diethyl 2-(cyclohexylmethylene) malonate (Entry 4). For comparison, the yields are 92%, 79%, 81%, and 79% when catalyzed by 10 mol % homogeneous frustrated Lewis pair catalyst, respectively. The comparison of the yields of these products indicates the MIL-101(Cr)-LP can be used as a porous frustrated Lewis pair catalyst and exhibits excellent catalytic performance in the frustrated Lewis pair-promoted hydrogenation.

TABLE 3
Catalysis Studies for Hydrogenation of Alkylidene Malonates

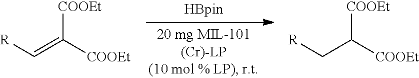

| | | Yield | Yield |
|---|---|---|---|
| Entry | Substrate | (FLP@MOF)^{a} | (FLP)^{b} |
| 1 | 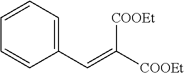 [SMILES: CCOC(=O)C(=Cc1ccccc1)C(=O)OCC] | 95% | 91% |
| 2 | 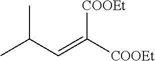 | 83% | 80% |
| 3 | 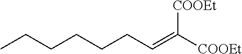 [SMILES: CCCCCCC=C(C(=O)OCC)C(=O)OCC] | 81% | 79% |
| 4 | 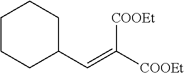 [SMILES: CCOC(=O)C(=CC1CCCCC1)C(=O)OCC] | 88% | 82% |

Reaction conditions: 20 mg MIL-101(Cr)-LP (10 mol % LP) for heterogeneous or DABCO/B(C₆F₅)₃ (10 mol %) for homogenous catalytic reaction, 0.12 mmol substrate, 3 mL toluene, 80° C., 24 hr, 60 bar H₂
^{a}Yields of isolated product.
^{b}Yield is relative to the substrate.

### Analysis of Tentative Catalytic Mechanism of MIL-101(Cr)-LP

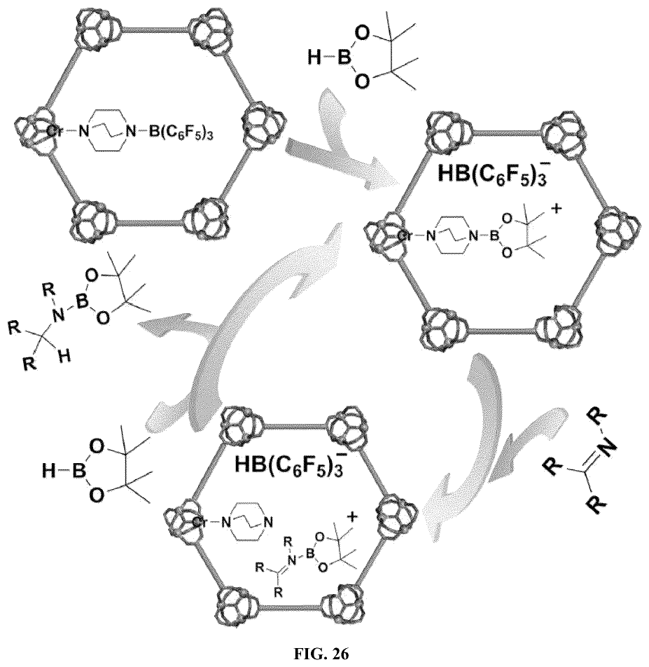

To investigate the impact of the porous framework structure on the catalysis performance of a frustrated Lewis pair, we examined the kinetics of the reduction of N-tert-butyl-1-phenylmethanimine with HBPin using heterogeneous MIL-101(Cr)-LP and homogeneous frustrated Lewis pair counterpart. The reactions catalyzed by both heterogeneous and homogeneous catalysts are on the same order of magnitude; MIL-101(Cr)-LP showed a slower reaction rate as expected due to the additional diffusion process needed for the substrates and products throughout the metal-organic framework pores. It is noteworthy that the similar kinetics behaviors of MIL-101(Cr)-LP and the homogeneous frustrated Lewis pair counterpart implies that MIL-101(Cr)-LP catalyst may share a similar reaction mechanism with that of a homogeneous reaction system. Without wishing to be bound by any one theory, the reaction is proposed to start with the generation of DABCO-borenium ion, then the borenium ion part transfers from DABCO-borenium ion to imine as shown in FIG. 26. The resulting B-activated iminium ion is then reduced by HBPin with assistance from the Lewis base of MIL-101(Cr)-LP. Reduction of imine-Bpin ion regenerates the borenium ion catalyst, which then reenters the catalytic cycle. The solid-state ¹⁹F NMR of MIL-101(Cr)-LP was used to investigate [HB(C₆F₅)₃] in the catalytic reactions. After 10 min of starting the reaction, the MIL-101(Cr)-LP was separated and dried for the solid-state ¹⁹F NMR measurement. The peaks from −125 to 170 ppm match the reported ¹⁹F NMR of [HB(C₆F₅)₃]⁻¹⁰. The results of solid-state ¹⁹F NMR confirmed the intermediate [HB(C₆F₅)₃]⁻ in the catalytic reactions, thus providing support for the above catalyst mechanism.

### Recycling Performance of MIL-101(Cr)-LP

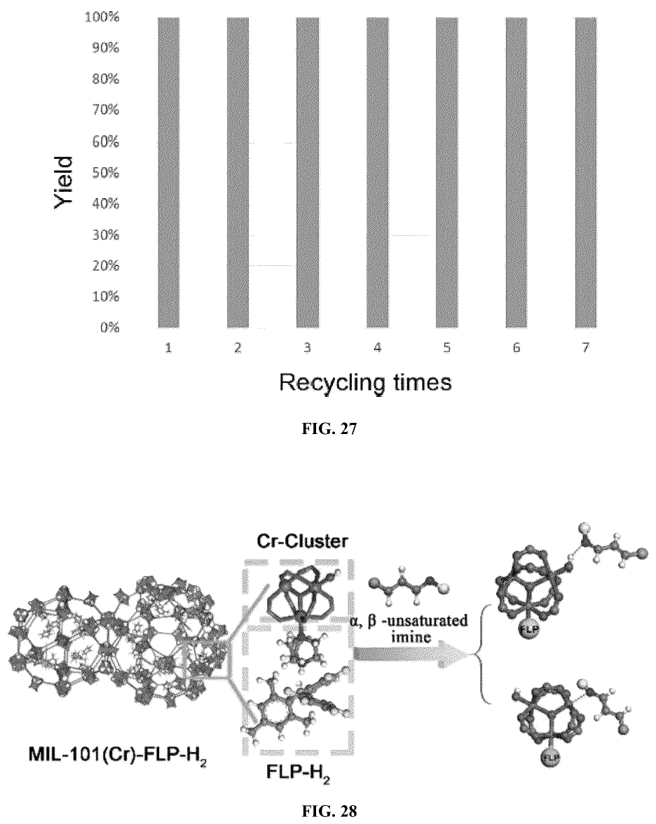

Given that the recyclability and long-term stability of the catalyst are the essential performance metrics for cost-effective industrial processes, these properties were investigated for MIL-101(Cr)-LP. Due to the existence of coordination bonds between the frustrated Lewis pair and metal-organic framework, the frustrated Lewis pair leaching is dispelled, given that there are no observable signals of frustrated Lewis pair in the liquid NMR spectrum of the supernatant after the reaction. Moreover, MIL-101(Cr)-LP can readily be recycled with the steady catalytic performance for at least seven cycles, thus highlighting the heterogeneous nature of the catalytic process (FIG. 27). The 1H NMR data of the decomposed MIL-101(Cr)-LP in 5 wt % NaOH D₂O solution after seven cycles of catalytic reaction matches the original MIL-101(Cr)-LP, thus reinforcing the idea that the frustrated Lewis pair is competently bound to the Cr sites. The robustness of the catalyst was further confirmed by the well-retained crystallinity and pore structure in MIL-101-LP after the catalytic reaction, as shown by PXRD and N2 adsorption studies, respectively.

### Materials

All chemical reagents were obtained from commercial sources and, unless otherwise noted, were used as received without further purification. Organic solvents used in this work were further purified and dried following standard procedures prior to use. All of the experiments for the frustrated Lewis pair were performed in the glove box.

### Synthesis of MIL-101(Cr)-LP

In the glove box, DABCO(2.5 mg, 0.022 mmol) was dissolved in anhydrous toluene with degassed MIL-101(Cr) (20 mg) soaked inside and, after 12 hr, the sample was centrifuged and washed with anhydrous toluene three times. The tris(pentafluorophenyl)borane (15.4 mg, 0.03 mmol) anhydrous toluene solution was added to the sample and then stirred for 12 hr. After that, the sample was centrifuged and washed with anhydrous toluene three times. The sample was then vacuumed for the following catalysis reaction.

### Catalytic Reactions

### General Procedure for a MIL-101(Cr)-LP-Catalyzed Imine Reduction Reaction (GP1)

In a N₂-filled glove box, MIL-101(Cr)-LP was dispersed into 3 mL of toluene in a 20 mL vial equipped with a small magnetic stir bar. HBPin was then added to the vial, and substrates were added to the vial after 10 min. The vial was capped and kept in the glove box at room temperature for the noted time. The catalyst was separated by centrifugation. The product was isolated as indicated in the Supplemental Information.

### General Procedure for a LP-Catalyzed Imine Reduction Reaction (GP2)

In a N₂-filled glove box, the frustrated Lewis pair was dispersed into 3 mL of toluene in a 20 mL vial equipped with a small magnetic stir bar. HBPin was then added to the vial, and substrates were added to the vial after 10 min. The vial was capped and kept in the glove box at room temperature for the noted time. The product was isolated as indicated in the Supplemental Information.

### General Procedure for a MIL-101(Cr)-LP-Catalyzed Alkylidene Malonate Hydrogenation Reaction (GP3)

In a N₂-filled glove box, MIL-101(Cr)-LP was dispersed into 2 mL of toluene in a 20 mL vial equipped with a small magnetic stir bar, and then the substrate toluene solution (1 mL) was added to the vial after stirring for 10 min. The resulting mixture was then transferred to the autoclave equipped with the magnetic stir bar. At last, the autoclave was pressurized with H₂ (60 bar) and heated to 80° C. for 24 hr. The catalyst was separated by centrifugation. The product was isolated as indicated in the Supplemental Information.

### General Procedure for a LP-Catalyzed Alkylidene Malonate Hydrogenation Reaction (GP4)

In a N₂-filled glove box, the frustrated Lewis pair was dispersed into 2 mL of toluene in a 20 mL vial equipped with a small magnetic stir bar, and then the substrates toluene solution (1 mL) was added to the vial after stirring for 10 min. The resulting mixture was then transferred to the autoclave equipped with the magnetic stir bar. The autoclave was then pressurized with H₂ (60 bar) and heated to 80° C. for 24 hr. The product was isolated as indicated in the Supplemental Information.

### Characterization

Elemental analysis was performed on a PerkinElmer 240 CHN elemental analyzer. Infrared spectra were recorded on a PerkinElmer UATR TWO FTIR spectrophotometer. PXRD measurements were recorded on a Bruker D8 Advance X-ray diffractometer with Cu Ka radiation. The simulated powder patterns were calculated with Mercury 2.0. The XPS data were collected on a PHI5000VersaProbe device, the microscan image and EDS elemental mapping analyses were tested on a ZEISS MERLIN Compact scanning and JEOL JSM-750OF electron microscope. TEM and HAADF-STEM were performed on a Tecnai G² F20 microscope (FEI). The NMR tests were performed on a Varian Unity Inova 400 spectrometer. Gas adsorption measurements were tested by a Micromeritics ASAP 2020 surface area and porosity analyzer.

### Example 2. Chemoselective Hydrogenation Using MIL-101(Cr)-LP

MIL-101(Cr) with the SBU of Cr₃(μ₃-O)(COO)₆(OH)—(H₂O)₂ was prepared according to the procedure previously reported and was dehydrated before the introduction of a frustrated Lewis pair. The Lewis base (LB) of 1,4-diazabicyclo[2.2.2]octane (DABCO) that features two binding nitrogen atoms, with one to anchor at the open CrIII and the other to interact with the Lewis acid (LA), was first grafted into metal-organic framework to form MIL-101(Cr)-LB. To stabilize the anchored frustrated Lewis pair, the Lewis acid B(C₆F₅)₂(Mes) was added to MIL-101(Cr)-LB, and then treated with H₂ to afford the HB(C₆F₅)₂(Mes)_/HDABCO+ pair anchored MIL-101-(Cr), which is denoted as MIL-101(Cr)-FLP-H₂. MIL-101(Cr)-FLP-H₂ (1.0 mmol FLP per 1 g MIL-101(Cr) or 0.34 FLP per unsaturated Cr site) was used in the following characterizations.

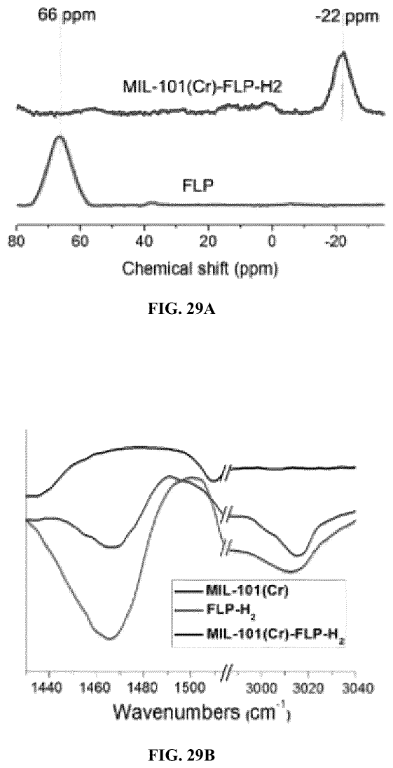

¹¹B NMR spectroscopy measurements were carried out to investigate the frustrated Lewis pair with activated hydrogen that is anchored on the pore wall of MIL-101(Cr)-FLP-H₂. As shown in FIG. 29A, the ¹¹B NMR spectrum of the mixture of B(C₆F₅)₂(Mes) and DABCO shows a peak at 66.1 ppm, indicative of an extremely weak interaction between the Lewis acid and Lewis base. After treating the Lewis acid with MIL-101(Cr)-LB in toluene under 10 bar H₂ atmosphere and at room temperature for 24 h, the solid-state ¹¹B NMR spectrum showed a distinct peak at −22.4 ppm, which is comparable to that of the homogeneous [HDABCO]+[HB(C₆F₅)₂(Mes)]⁻ (δ=−22.5 ppm) and related [HB(C₆F₅)₂(Mes)][LB] system (δ=−22.1 ppm), thus confirming the formation of the presumed ammonium hydridoborate of the frustrated Lewis pair. Furthermore, the peaks from the solid-state ¹⁹F NMR spectrum of MIL-101(Cr)-FLP-H₂ are consistent with the formation of a tetracoordinate anionic borate, further confirming the existence of a frustrated Lewis pair ammonium hydridoborate within the metal-organic framework.

The porosity and phase purity of MIL-101(Cr)-LB and MIL-101(Cr)-FLP-H₂ were investigated by N₂ gas sorption at 77 K and powder X-ray diffraction (PXRD) measurements, respectively. The PXRD patterns of MIL-101(Cr)-LB and MIL-101(Cr)-FLP-H₂ are in good agreement with the calculated ones and those of the pristine MIL-101(Cr), indicating the retention of the framework's structural integrity during the stepwise loading process. The N₂ sorption studies indicated that in comparison with pristine MIL-101(Cr), there is a steady decrease in the BET surface area (from 2724 to 2196 and 1112 m²g⁻¹) and reduction in pore sizes for MIL-101(Cr)-LB and MIL-101(Cr)-FLP-H₂ due to the Lewis base and frustrated Lewis pair molecules grafted onto the pore wall of MIL-101(Cr).

The Fourier transform infrared (FT-IR) spectroscopy analysis was employed to verify the association of the frustrated Lewis pair and MIL-101(Cr) framework. The spectrum of MIL-101(Cr)-FLP-H₂ shows the characteristic peaks from Lewis base and Lewis acid. As shown in FIG. 29B, the presence of a new band at high wavenumbers (3000-3030 cm-1) is due to the methyl C—H stretching vibrations of LA, and the new band around 1466 arises from the aliphatic C—H stretching vibrations of LB. The above notable new bands of MIL-101(Cr)-FLP-H₂ further suggest the existence of the FLP-H₂ inside the metal-organic framework.

The coordination interaction between frustrated Lewis pair and the open Cr^{III} sites in MIL-101(Cr) was investigated by X-ray photoelectron spectroscopy (XPS) spectra of MIL-101(Cr) and MIL-101(Cr)-FLP-H₂. The Cr(2p) spectrum of MIL-101-(Cr)-FLP-H₂ is notably different from the Cr(2p) spectrum of MIL-101(Cr). The Cr(2p_{1/2}) and Cr (2p_{3/2}) peaks of MIL-101(Cr)-FLP-H₂ are shifted by around 0.58-2 eV toward higher binding energies, compared to those of MIL-101(Cr). Such shifts indicate a change in the electron density of Cr^{III}, which can be attributed to the interaction between Cr and DABCO. The survey spectrum of MIL-101(Cr)-FLP-H₂ indicates the presence of the F and N elements of the frustrated Lewis pair molecule within the metal-organic framework.

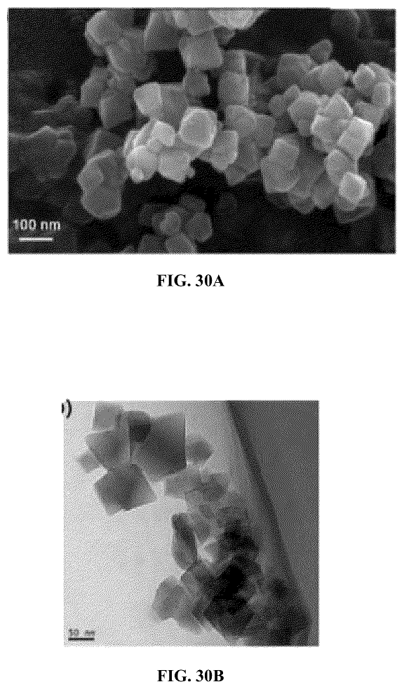

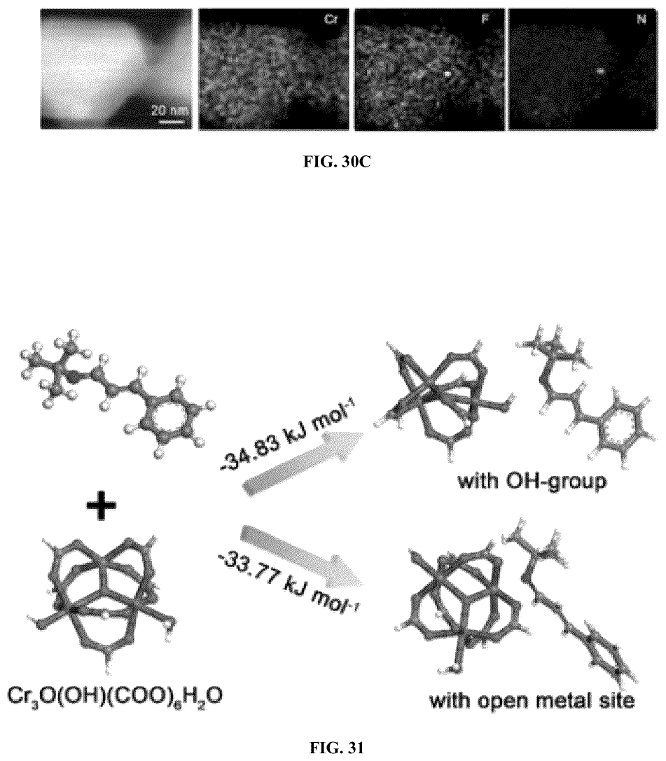

The morphology of MIL-101(Cr)-FLP-H₂ was investigated by scanning electron microscopy (SEM) and transmission electron microscopy (TEM). As shown in FIGS. 30A and 30B, the SEM and TEM images exhibited regular octahedral crystals of MIL-101(Cr)-FLP-H₂ with an average diameter of about 100 nm. To investigate the distribution of FLP-H₂ in MIL-101(Cr)-FLP-H₂, high-angle annular darkfield scanning transmission electron microscopy (HAADF-STEM) and energy dispersive X-ray spectroscopy (EDS) elemental mapping analyses were conducted. As presented in FIG. 30C, the Cr, F, and N elements are evenly distributed inside the octahedral crystal of MIL-101(Cr)-FLP-H₂, suggesting the integration of the frustrated Lewis pair inside the pores of the metal-organic framework. These results indicated that the frustrated Lewis pair was homogeneously distributed in the MIL-101(Cr) pores without the presence of accumulation in particular regions.

To investigate the optimal loading amount for catalyzing the imine reduction reactions, we prepared a series of frustrated Lewis pair-impregnated metal-organic frameworks with different frustrated Lewis pair uptake amounts, (MIL-101(Cr)-FLP-H₂-1 (0.5 mmol FLP per 1 g MIL-101(Cr)) and MIL-101(Cr)-FLP-H₂-2 (0.75 mmol FLP per 1 g MIL-101(Cr))). The loading amount of frustrated Lewis pair in the above samples was quantified with 1H NMR spectroscopy by decomposing the frustrated Lewis pair-impregnated metal-organic framework in 10 wt % NaOH deuterium oxide solution. The catalytic performances of the MIL-101(Cr)-FLP-H₂ catalysts were evaluated by exposing N-tert-butyl-1-phenylmethanimine to 20 mg catalyst in toluene, resulting in the reduction to N-benzyl-tert-butylamine at 10 bar H₂ atmosphere and room temperature after 48 h. Along with the increase of the frustrated Lewis pair loading amount, the catalytic yield for the above reaction increased. The frustrated Lewis pair-impregnated metal-organic framework with the lowest amount of frustrated Lewis pair, MIL-101(Cr)-FLP-H₂-1, only gave 36% yield, whereas the highest uptake amount of frustrated Lewis pair loaded MIL-101(Cr)-FLP-H₂ exhibited complete conversion. Therefore, MIL-101(Cr)-FLP-H₂ was chosen for the following catalysis studies. The control experiments using pristine MIL-101(Cr), MIL-101(Cr)-LB, and MIL-101(Cr)/Lewis acid were conducted, however no reduction product was detected in the reaction solvent even after 72 h, suggesting MIL-101(Cr), MIL-101(Cr)-LB, and MIL-101(Cr)/Lewis acid are inactive for the imine reduction reaction.

Imine compounds with different substituting groups were employed to investigate the catalytic property of heterogeneous MIL-101(Cr)-FLP-H₂. The reaction yields catalyzed by MIL-101(Cr)-FLP-H₂ from related imine compounds are 68% for N-benzylideneaniline, 91% for N-benzylidene-1-phenylmethanamine, and 58% for acridine. The catalysis results reveal that the steric effect close to the N atoms can affect the catalytic performance. The steric effect could be presumably owing to the confinement effect imparted by the porous structure of the metal-organic framework, which restricts the accessibility of buried C═N double bonds to the frustrated Lewis pair active centers that are anchored on the pore walls of the metal-organic framework. Along with the increase in size of the imine substrate, the reduction reaction yield decreased. The reaction yield catalyzed by MIL-101(Cr)-FLP-H₂ for N-benzhydryl-1-phenylmethanimine is 21% and none of the product could be observed for N-benzhydryl-1,1-diphenylmethanimine even after 72 h. Considering the existence of the coordinated frustrated Lewis pair on the window, the size of the window (with diameters of ca. 1.0 nm) is smaller than the diameter of -benzhydryl-1,1-diphenylmethanimine molecule (with diameters of 1.3 nm), thus the large imine molecule could not enter into the pores of MIL-101(Cr)-FLP-H₂. Therefore, the observed size selectivity performance of MIL-101(Cr)-FLP-H₂ can be attributed to the restriction of the window structure in the framework.

Considering the interesting catalytic performance of MIL-101(Cr)-FLP-H₂ in the above imine reduction reactions, we decided to examine MIL-101(Cr)-FLP-H₂ for selective catalytic hydrogenation of the α,β-unsaturated imine compounds directly using hydrogen gas, which has barely been explored in both homogeneous and heterogeneous systems. Interestingly, for the substrate N-(tert-butyl)-3-phenylprop-2-en-1-imine, both imine and alkene double bonds were reduced in the case of a homogeneous frustrated Lewis pair, and no N-(tert-butyl)-3-phenylprop-2-en-1-amine can be observed after the catalytic reaction. However, the heterogeneous MIL-101(Cr)-FLP-H₂ shows a yield of 87% N-(tert-butyl)-3-phenylprop-2-en-1-amine for N-(tert-butyl)-3-phenylprop-2-en-1-imine under the same reaction conditions. This excellent selectivity could be attributed to the hydrogen bonding between the N atoms of the imines and the —OH groups from the framework and/or the interaction between the N atoms of the imines and remaining open metal sites. The α,β-unsaturated imine compounds with different substituted groups were employed to further investigate the chemoselective catalysis performance of the heterogeneous MIL-101(Cr)-FLP-H₂. As presented in Table 1, the reaction yields catalyzed by MIL-101(Cr)-FLP-H₂ from the related α,β-unsaturated imine compounds are 100% for N,3-diphenylprop-2-en-1-imine, 91% for N-(4-methoxyphenyl)but-2-en-1-imine, 81% for N-(tert-butyl)but-2-en-1-imine, 100% for N-(4-methoxyphenyl)-3-phenylprop-2-en-1-imine, and 93% for N-phenylbut-2-en-1-imine. The high yields of these products highlight that MIL-101(Cr)-FLP-H₂ can serve as a porous frustrated Lewis pair catalyst with excellent catalytic performance in the chemoselective hydrogenation reactions, which has not been achieved previously for any frustrated Lewis pair-based catalyst.

TABLE 4
Catalytic Hydrogenation of α,β-Unsaturated Imine Compounds^{a}

| Entry | Substrate | Product | Yield (%)^{b} |
|---|---|---|---|
| 1 | 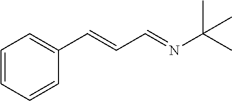 | 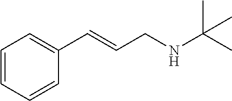 | 0 |
| 2 | 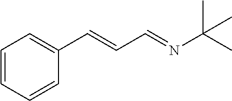 | 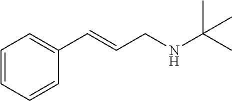 | 87 |
| 3 | 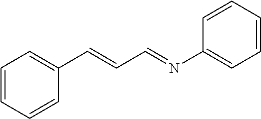 [SMILES: C(=C\c1ccccc1)/C=N/c1ccccc1] | 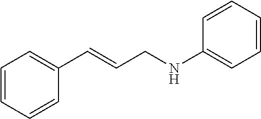 [SMILES: C(=C\c1ccccc1)/CNc1ccccc1] | 100 |
| 4 | 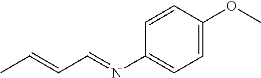 | 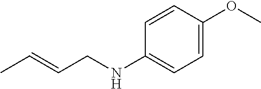 | 91 |
| 5 | 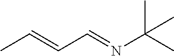 | 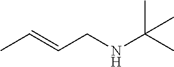 | 81 |
| 6 | 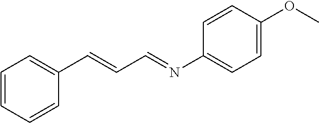 [SMILES: COc1ccc(/N=C/C=C/c2ccccc2)cc1] | 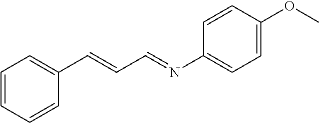 [SMILES: COc1ccc(/N=C/C=C/c2ccccc2)cc1] | 100 |
| 7 | 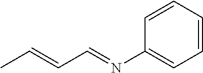 | 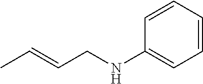 | 93 |

^{a}Reaction conditions: 20 mg (10 mol % FLP) MIL-101(Cr)-FLP-H₂, 0.13 mmol substrate, 3 mL toluene, room temperature, 48 h.
^{b}Reaction conditions: 10 mol % FLP, 0.13 mmol substrate, 3 mL toluene, room temperature, 40 h.

On the basis of the reported homogenous frustrated Lewis pair catalyzed hydrogenation reactions and the solid-state ¹⁹F NMR spectroscopy results for MIL-101(Cr)-FLP-H₂, the tentative catalysis mechanism of MIL-101(Cr)-FLP-H₂-catalyzed imine reduction reactions is proposed. The process is initiated by the reaction between MIL-101(Cr)-FLP-H₂ and the imine substrate. The [HBMes-(C₆F₅)₂] reduces the imine substrate and then converts to BMes(C₆F₅)₂, while the MIL-101(Cr)-[LBH]⁺ converts into MIL-101(Cr)-LB. Subsequently, the MIL-101(Cr)-LB and BMes(C₆F₅)₂ react with hydrogen to regenerate the MIL-101(Cr)-FLP-H₂ and complete the catalysis cycle.

Density functional theory (DFT) calculations were employed to understand the tentative mechanism of the chemoselective hydrogenation of α,β-unsaturated imine compounds. As shown in FIG. 31, the unsaturated Cr^{III} sites and the hydroxy group in Cr₃O(OH)(COO)₆H₂O trimers of MIL-101 can serve as “active” sites that interact with the C═N group of N-(tert-butyl)-3-phenylprop-2-en-1-imine. The calculated adsorption energy of N-(tert-butyl)-3-phenylprop-2-en-1-imine over the unsaturated Cr^{III} sites and the hydroxy group in Cr³O(OH)(COO)₆H₂O trimers is −33.77 and −34.83 kJ mol⁻¹, respectively; that is, binding between the trimers and N-(tert-butyl)-3-phenylprop-2-en-1-imine through N . . . HO and N . . . Cr is thermodynamically favored, with the interaction between N-(tert-butyl)-3-phenylprop-2-en-1-imine and hydroxy group in Cr₃O(OH)(COO)₆H₂O trimers being slightly stronger. Therefore, different from the homogenous catalyst, the metal-organic framework catalyst can selectively reduce the C═N bond in α,β-unsaturated imine compounds. These results support the experimental observation that the —OH groups and remaining open Cr^{III} sites in MIL-101(Cr) preferentially interact with the C═N group (rather than the C═C group) of N-(tert-butyl)-3-phenylprop-2-en-1-imine thus to activate it, giving rise to the improved selectivity for the formation of product N-(tert-butyl)-3-phenylprop-2-en-1-amine.

In industrial processes, the recyclability and long-term stability of the catalyst are of utmost importance. Based on this consideration, we investigated the stability and recycling performance of MIL-101(Cr)-FLP-H₂. MIL-101(Cr)-FLP-H₂ can be readily recycled with great catalytic performance, and the yield of the catalysis reaction can maintain 100% percent even at the fifth recycling experiment. The ¹H NMR data of the decomposed MIL-101(Cr)-FLP-H₂ in 10 wt % NaOH deuterium oxide solution after five cycles of catalytic reaction matches the original MIL-101(Cr)-LB, thus reinforcing the idea that the Lewis base portion of the frustrated Lewis pair is competently bound to the CrIII sites. The robustness of the catalyst was further confirmed by the well-retained crystallinity and pore structure in MIL-101(Cr)-FLP-H₂ after the catalytic reaction, as evidenced by PXRD and N₂ adsorption studies, respectively.

### Materials and General Methods

All chemical reagents were obtained from commercial sources and, unless otherwise noted, were used as received without further purification. Organic solvents used in this work were further purified and dried following standard procedures prior to use. All of the experiments about Lewis pair were performed in the glove box. Elemental analysis was performed on a Perkin-Elmer 240 CHN elemental analyzer. IR spectra were recorded on a Perkin Elmer UATR TWO FT-IR spectrophotometer. Powder X-ray diffraction measurements (PXRD) were recorded on a Bruker D8 Advance X-ray diffractometer using Cu Ka radiation. The simulated powder patterns were calculated by using Mercury 2.0. The X-ray photoelectron spectroscopy (XPS) data were collected at PHI5000VersaProbe device, the micro scan image and energy dispersive X-ray spectroscopy (EDS) elemental mapping analyses were tested on ZEISS MERLIN Compact scanning and JEOL JSM-750OF electron microscope (SEM). TEM, HAADF-STEM was performed on Tecnai G² F20, FEI, Hillsboro, OR, USA. The NMR tests were performed on the Varian Unity Inova 400 spectrometer. Gas adsorption measurement was tested by Micromeritics ASAP 2020 surface area and porosity analyzer.

### Synthesis of MIL-101(Cr)

Cr (NO₃)₃·9H₂O (1.0 g, 2.5 mmol), terephthalic acid (0.46 g, 2.5 mmol), and deionized water (10 mL) were blended and briefly sonicated resulting in a dark blue-colored suspension. The suspension was placed in a Teflon-lined autoclave bomb and kept in an oven at 218° C. for 18 h. After the bomb cooled down to room temperature, the resultant fine green MIL-101 crystals were separated from water using a centrifuge and washed with water, methanol and acetone. The suspension in acetone was centrifuged and separated, the solids were placed in N,N-dimethylformamide (10 mL) and the suspension was sonicated for 10 min and then kept at 70° C. overnight. The resulting solid was separated by centrifugation, washed with methanol and acetone repeatedly, dried at 75° C. overnight, and then was degassed on the degas station of the surface area and porosity analyzer under vacuum (1×10⁻³ mmHg) and 120° C. for 12 h to give dehydrated MIL-101(Cr).

### Synthesis of MesB(C₆F₅)₂

Magnesium turnings (1 g, 41.7 mmol) were suspended in 20 mL ether and a small amount of iodine added followed by the addition of a small amount of BrC₆F₅ (0.5 mL) resulting in a turbid grey mixture. To start the reaction, the mixture was heated and then the remaining BrC₆F₅ (9 g, 4.5 mL) was added slowly. Once the addition of BrC₆F₅ was complete, the resulting mixture was stirred for 1 h at room temperature, resulting in a black solution. The above C₆F₅MgBr ether solution was added dropwise to an Et₂O solution (20 mL) of BF3×OEt2 (1.0 ml, 1.20 g) at 0° C. The reaction mixture was stirred at 0° C. for additional 3 hours then an Et₂O solution (10 mL) of C₆H₂Me₃MgBr was added dropwise to the reaction mixture at 0° C. The reaction mixture was stirred at 0° C. for two more hours, then warmed up to room temperature, and stirred overnight. Solvents were removed in vacuo and the residue was pulverized in the glovebox. The resulting brown powder put into two-fold sublimation (130° C. and 1×10⁻³ mbar) whereupon the pure MesB(C₆F₅)₂ (1.8 g) was collected as the white needles.

¹H-NMR (300 MHZ, C₆D₆) δ=6.68 (s, 2H); 2.11 (s, 3H); 1.99 (s, 6H) ppm; ¹⁹F-NMR (282 MHZ, C₆D₆) δ=−129.6; −145.3; −161.2 ppm; ¹³C-NMR (75 MHZ, C₆D₆) δ=149.8; 146.5; 142.7; 140.4; 139.3; 137.8; 135.9; 128.5; 22.3; 21.2 ppm; ¹⁰B-NMR (43.0 MHZ, C₆D₅CD₃) δ=69.7 ppm.

### Synthesis of FLP: 1,4-diazabiyclo[2.2.2]-octane bis(pentafluorophentl) mesitylborane

In the glove box, MesB(C₆F₅)₂ (100 mg, 0.2 mmol) and 1,4-diazabicyclo[2.2.2]-octane (DABCO) (22.4 mg, 0.2 mmol) were dissolved in toluene and stirred 1 hour. Then the solvent was vacuumed to give the FLP product for the following characterization and catalysis reactions. ¹H NMR (CD₂Cl₂, 400 MHZ) δ 2.02 (s, 6H, CH₃), 2.12 (s, 3H, CH₃), 2.50 (s, 12H, NCH₂), 6.66 (s, 2H, PhH).

### Synthesis of MIL-101(Cr)-FLP-H₂-n

General Process: In the glove box, DABCO was dissolved in toluene with degassed MIL-101(Cr) (20 mg) soaked inside, after 12 hours, the sample was centrifuged and washed with dry toluene for 3 times. The resultant MIL-101(Cr)-LB-n was added to the tris(pentafluorophenyl)borane toluene solution, and then transferred to the autoclave equipped with the magnetic stir bar. The autoclave was pressurized with H₂ (10 bar) and room temperature for 24 h. After that, the sample was centrifuged and washed with dry toluene for three times. At last, the sample was vacuumed for the following catalysis reaction.

### Synthesis of MIL-101(Cr)-FLP-H₂-1 (0.5 mmol/g)

The reaction was carried out following a general process from DABCO (1.3 mg, 0.012 mmol) and tris(pentafluorophenyl)borane (4.6 mg, 0.01 mmol), the sample was centrifuged and the solvent was removed, then the powder was washed with toluene for three times. At last, the sample was vacuumed for the following catalysis reaction.

### Synthesis of MIL-101(Cr)-FLP-H₂-2 (0.75 mmol/g)

The reaction was carried out following a general process from DABCO (1.6 mg, 0.015 mmol) and tris(pentafluorophenyl)borane (7 mg, 0.015 mmol), the sample was centrifuged and the solvent was removed, then the powder was washed with toluene for three times. At last, the sample was vacuumed for the following catalysis reaction.

### Synthesis of MIL-101(Cr)-FLP-H₂ (1 mmol/g)

The reaction was carried out following general process from DABCO (2.5 mg, 0.022 mmol) and tris(pentafluorophenyl)borane (9.28 mg, 0.02 mmol), the sample was centrifuged and the solvent was removed, then the powder was washed with toluene three times. At last, the sample was vacuumed for the following catalysis reaction.

### Example 3. Hydrogen Storage

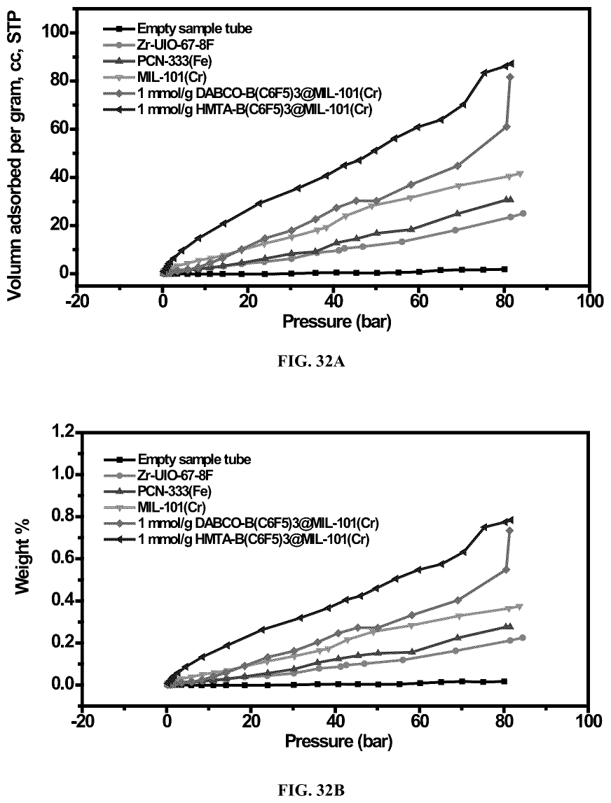

Compositions described herein were evaluated for hydrogen storage and compared to controls (blank sample and MOFs without frustrated Lewis pairs). The results are provided in FIGS. 32A-B. The results indicate that DABCO-B(C₆F₅)₃@MIL-101(Cr) and HMTA-B(C₆F₅)₃@MIL-101(Cr) had significantly increased hydrogen adsorption at high pressure hydrogen storage (up to 80 bars) at room temperature.

Publications cited herein are hereby specifically incorporated by reference in their entireties and at least for the material for which they are cited.

While it should be understood that while the present disclosure has been provided in detail with respect to certain illustrative and specific aspects thereof, it should be considered limited to such, as numerous modifications are possible without departing from the broad spirit and scope of the present disclosure as defined in the appended claims. It is, therefore, intended that the appended claims cover all such equivalent variations as fall within the true spirit and scope of the invention.

1. A composition comprising:
a porous material having at least one nanospace comprising a porous metal-organic framework (MOF) or a porous organic polymer (POP); and
a frustrated Lewis pair comprising a Lewis base and a Lewis acid;
wherein the frustrated Lewis pair is contained within at least one nanospace of the porous material.

2-6. (canceled)

7. The composition of claim 1, wherein the metal-organic framework comprises one or more metal ions and one or more organic linker ligands.

8. The composition of claim 7, wherein the one or more metal ions are an ion of a metal selected from Mg, Ca, Sr, Ba, Sc, Y, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Hg, Al, Ga, In, TI, Si, Ge, Sn, Pb, As, Sb, and Bi, or combinations thereof.

9. The composition of claim 7, wherein the one or more organic linker ligands are selected from a polycarboxylate ligand, a polypyridyl ligand, a polycyano ligand, a polyphosphate ligand, a polyhydroxyl ligand, a polysulfonate ligand, a polyimidazolate ligand, a polytriazolate ligand, a polytetrazolate ligand, and a polypyrazolate ligand, or combinations thereof.

10. The composition of claim 7, wherein the one or more organic linker ligands are selected from: 1,2,4,5-tetrakis(4-carboxyphenyl)benzene; 1,3,5-tris(4′-carboxy[1,1′-biphenyl]-4-yl)benzene; 1,3,5-tris(4-carboxyphenyl)benzene; 2,5-dihydroxyterephthalic acid; 2,6-naphthalenedicarboxylic acid; 2-hydroxyterephthalic acid; 2-methylimidazole; 3,3′,5,5′-tetracarboxydiphenylmethane; 4,4′,4″-s-triazine-2,4,6-triyl-tribenzoic acid; 9,10-anthracenedicarboxylic acid; biphenyl-3,3′,5,5′-tetracarboxylic acid; biphenyl-3,4′,5-tricarboxylic acid; imidazole, terephthalic acid; trimesic acid; [1,1′:4′,1″]terphenyl-3,3′,5,5′-tetracarboxylic acid; or combinations thereof.

11. The composition of claim 1, wherein the metal-organic framework comprises a chromium metal-organic framework, an iron metal-organic framework, or a zirconium metal-organic framework.

12. The composition of claim 1, wherein the metal-organic framework is MIL-101(Cr), PCN-333(Cr), PCN-333(Fe), Tb-mesoMOF, MOF-74-III, PCN-777, PCN-69, Zr-UiO-68, Zr-UIO-67-8F, UiO-68, MOF818, FDM-3, Tb-TATB, MIL-101-4F, or MIL-101-Br(Cr)

13. (canceled)

14. The composition of claim 1, wherein the at least one nanospace has an average window size less than the size of the frustrated Lewis pair.

15-16. (canceled)

17. The composition of claim 1, wherein the Lewis acid is a compound of Formula I: 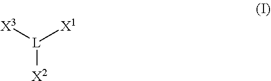
wherein:
L is a group 13 element;
X¹ and X² are each independently selected from alkyl, cycloalkyl, aryl, or heteroaryl, each of which may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl; and
X³ is selected from hydrogen, halogen, alkyl, cycloalkyl, aryl, or heteroaryl, each of which except for hydrogen and halogen may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SO₂aryl, or SO₂alkyl.

18. The composition of claim 1, wherein the Lewis acid is a compound of Formula II: 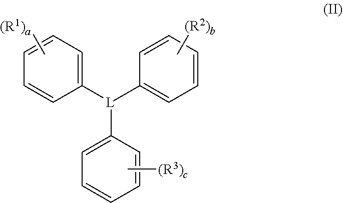
wherein:
L is a group 13 element;
R¹ R², and R³ are independently selected at each occurrence from amino, cyano, halo, hydroxy, nitro, phenyl, C₁₋₆ alkyl, C₃₋₆ cycloalkyl, C₁₋₆ fluoroalkyl, C₁₋₆ chloroalkyl, C₁₋₆ alkoxy, and C₁₋₆ alkylphenyl; and
a, b, and c are independently selected from 0, 1, 2, 3, 4, or 5.

19. The composition of claim 1, wherein the Lewis acid is a compound of Formula III: 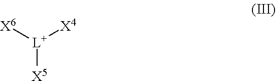
wherein:
L⁺ is a group 13 ion;
X⁴ and X⁵ are each independently selected from alkyl, cycloalkyl, haloalkyl, aryl, or heteroaryl, wherein each X⁴ and X⁵ may be optionally substituted with at least one substituent selected from cyano, halo, hydroxyl, nitro, optionally substituted mesityl, C₁₋₆ alkyl, C₃₋₆ cycloalkyl, C₁₋₆ alkoxy, phenyl, C₁₋₆ alkylphenyl, heterocyclyl, C₁₋₆ alkylheterocyclyl, SO₂aryl, and SO₂alkyl; or
X⁴ and X⁵ are brought together with the atom to which they are attached to form a four to ten atom saturated or unsaturated monocyclic or bicyclic ring which may optionally comprises one or two additional heteroatoms selected from nitrogen, oxygen, and sulfur and which may be optionally substituted with one or more substituents selected from amino, cyano, halo, nitro, trifluoromethyl, C₁₋₆ alkyl, C₁₋₆ alkoxy, —S(C₁₋₆ alkyl), —S(O)(C₁₋₆ alkyl), or —S(O)₂(C₁₋₆ alkyl); and
X⁶ is an electron pair donor ligand coordinated to L⁺.

20-21. (canceled)

22. The composition of claim 1, wherein the Lewis base is a compound of Formula IV: 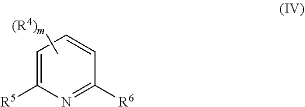
wherein:
R⁴, R⁵, and R⁶ are each independently selected at each occurrence from amino, cyano, halo, nitro, trifluoromethyl, C₁₋₆ alkyl, aryl, C₁₋₆ alkoxy, —S(C₁₋₆ alkyl), —S(O)(C₁₋₆ alkyl), and —S(O)₂(C₁₋₆ alkyl); and
m is 0, 1, 2, or 3.

23. The composition of claim 1, wherein the Lewis base is a compound of Formula V: 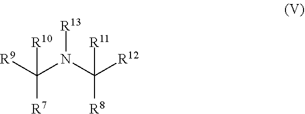
wherein:
R⁷ and R⁸ are each independently optionally substituted C₁₋₆ alkyl; or
R⁷ and R⁸ may be brought together with the atoms to which they are attached to form a five, six, or seven membered heterocyclic ring which may comprise one or two additional heteroatoms selected from nitrogen, oxygen or sulfur and which may be optionally substituted with one or more substituent groups selected from amino, cyano, halo, nitro, trifluoromethyl, C₁₋₆ alkyl, C₁₋₆ alkoxy, —S(C₁₋₆ alkyl), —S(O)(C₁₋₆ alkyl), or —S(O)₂(C₁₋₆ alkyl);
R⁹, R¹⁰, R¹¹, and R¹² are selected from hydrogen, alkyl, cycloalkyl, aryl, or heteroaryl, each which may be optionally substituted with one or more substituents selected form amino, cyano, halo, nitro, trifluoromethyl, C₁₋₆ alkyl, C₁₋₆ alkoxy, —S(C₁₋₆ alkyl), —S(O)(C₁₋₆ alkyl), or —S(O)₂(C₁₋₆ alkyl); and
R¹³ is hydrogen, C₁₋₃ alkyl, aryl, or heteroaryl.

24-27. (canceled)

28. The composition of claim 1, wherein the Lewis base comprises a bridging heterocyclic compound.

29-31. (canceled)

32. The composition of claim 1, wherein the Lewis acid and Lewis base of the frustrated Lewis pair are covalently linked by a divalent organic linker.

33-41. (canceled)

42. The composition of claim 1, wherein the Lewis acid of the frustrated Lewis pair is covalently bound to at least one or more organic linker ligands.

43. The composition of claim 1, wherein the Lewis base of the frustrated Lewis pair is covalently bound to at least one or more organic linker ligands.

44-66. (canceled)

67. A fuel cell comprising the composition of claim 1.

68. A process for hydrogenation or hydrogenolysis of an organic compound with at least one point of unsaturation, comprising:
contacting the organic compound with a composition of claim 1 in the presence of hydrogen gas.

69-75. (canceled)

76. A composition comprising:
a porous material having at least one nanospace comprising a porous metal-organic framework (MOF), the metal-organic framework comprising one or more metal ions and one or more organic linker ligands, the one or more metal ions selected from the group consisting of Mg, Ca, Sr, Ba, Sc, Y, Ti, Zr, Hf, V, Nb, Ta, Cr, Mo, W, Mn, Re, Fe, Ru, Os, Co, Rh, Ir, Ni, Pd, Pt, Cu, Ag, Au, Zn, Cd, Hg, Al, Ga, In, TI, Si, Ge, Sn, Pb, As, Sb, and Bi, and combinations thereof, and the one or more organic linker ligands being selected from the group consisting of a polycarboxylate ligand, a polypyridyl ligand, a polycyano ligand, a polyphosphate ligand, a polyhydroxyl ligand, a polysulfonate ligand, a polyimidazolate ligand, a polytriazolate ligand, a polytetrazolate ligand, and a polypyrazolate ligand, and combinations thereof; and
a frustrated Lewis pair comprising a Lewis base and a Lewis acid, wherein the Lewis acid is a compound of Formula I: 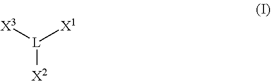
wherein:
L is a group 13 element;
X¹ and X² are each independently selected from alkyl, cycloalkyl, aryl, or heteroaryl, each of which may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SOzaryl, or SOzalkyl; and
X³ is selected from hydrogen, halogen, alkyl, cycloalkyl, aryl, or heteroaryl, each of which except for hydrogen and halogen may be optionally substituted with at least one substituent selected from cyano, halo, hydroxyl, nitro, mesityl, substituted mesityl, alkyl, cycloalkyl, alkoxy, phenyl, alkylphenyl, heterocyclyl, alkylheterocyclyl, SOzaryl, or SOzalkyl, and wherein the Lewis base is a compound of Formula IV: 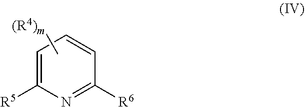
wherein:
R⁴, R⁵, and R⁶ are each independently selected at each occurrence from amino, cyano, halo, nitro, trifluoromethyl, C₁₋₆ alkyl, aryl, C₁₋₄ alkoxy, —S(C₁₋₆ alkyl), —S(O)(C₁₋₄ alkyl), and —S(O)₂(C₁₋₆ alkyl); and
m is 0, 1, 2, or 3, and
wherein the frustrated Lewis pair is contained within at least one nanospace of the porous material.
